(19) United States
(12) Patent Application Publication
(10) Pub. No.: US 2020/0392219 A1
(43) Pub. Date: Dec. 17, 2020

(54) EXTRACELLULAR VESICLES COMPRISING MEMBRANE-TETHERED TGF-BETA, COMPOSITIONS AND METHODS OF USE THEREOF

(71) Applicant: TRUSTEES OF TUFTS COLLEGE, MEDFORD, MA (US)

(72) Inventor: ANDREW M. HOFFMAN, MEDFORD, MA (US)

(73) Assignee: TRUSTEES OF TUFTS COLLEGE, MEDFORD, MA (US)

(21) Appl. No.: 16/611,099

(22) PCT Filed: May 7, 2018
(86) PCT No.: PCT/US2018/031386

§ 371 Date: Nov. 5, 2019

Related U.S. Application Data
(60) Provisional application No. 62/502,974, filed on May 8, 2017.

(51) Int. Cl. C07K 16/22; C12N 5/0775; C07K 14/705; C07K 14/54; C07K 14/71; C12N 5/00

(52) U.S. Cl. CPC C07K 16/22; C12N 5/0665; C07K 14/70578; C07K 14/54; C07K 14/70596; C12N 2501/15; C07K 14/71; C12N 5/0031; C12N 2510/00; C12N 2506/1346; C07K 14/70517

(57) ABSTRACT

Provided are mesenchymal stromal cell (MSC)-derived extracellular vesicles (EV) having tethered (membrane-bound) TGF-β (MSC-derived membrane-tethered TGF-β EV), and compositions containing such EV for use as therapeutics and immunomodulatory agents. Provided also are diagnostic methods and methods of assessing or monitoring disease status and/or progression in patients using membrane-tethered TGF-β derived from a variety of cell sources that serve as detectable, quantifiable biomarkers in biological samples. The MSC-derived membrane-tethered TGF-β EV can also be used to deliver various bioactive agents to a target cell or tissue for treating various diseases. The level of TGF-β tethered to the membrane of the EV can also be modified or manipulated in vitro or ex vivo. Such modified MSC-derived membrane-tethered TGF-β EV are useful as immunotherapeutic agents in the treatment or management of certain diseases, particularly those involving inflammation, autoimmunity, transplant rejection and cancer.

### STATEMENT OF PRIORITY

This application claims benefit of and priority to U.S. Provisional Application No. 62/502,974, filed on May 8, 2017, the contents of which are incorporated herein in their entirety.

### BACKGROUND OF THE INVENTION

Transforming growth factor-beta (TGF-β), as well as other immunomodulatory proteins, is associated with regulation of numerous biological functions such as cell proliferation, survival and differentiation. A variety of cell types of different lineages release TGF-β as a soluble mediator. Soluble TGF-β in plasma or tissues has been associated with protective responses in inflammatory diseases, particularly after acute insult, e.g., following cardiac-related events. However, the assessment of soluble TGF-β, or other immunomodulatory proteins, as an immunomodulator or reliable indicator of the quality or extent of patient response to disease has been fraught with dichotomous or variable results. Thus, there is a need for methods and improved methods of identifying and quantifying biologically relevant levels of immune modulators such as TGF-β and forms thereof in patients with disease, particularly, for assessing various treatment parameters, such as disease status, patient response to therapy and modification or adjustment of disease treatment, as well as for designing new therapies to treat patients who suffer from a variety of diseases.

### SUMMARY OF THE INVENTION

The present invention generally provides extracellular vesicles (EV), originating from a cell source, e.g., a cancer associated cell (e.g., fibroblast-like cell, cancer-associated fibroblast), dendritic cell, stromal cell, stromal stem cell, or mesenchymal stromal cell

(MSC), or a tissue or organ source, and having tethered (i.e., membrane bound) to the EV membrane an immunomodulatory agent, e.g., a protein or polypeptide. In a specific embodiment, the agent tethered to the EV membrane is a protein in the transforming growth factor-beta (TGF-β) family of immunomodulatory proteins, e.g., TGF-β or an isoform of TGF-β, namely, TGF-β1, TGF-β2, TGF-β3, and TGF-β4. In an embodiment, TGF-β is tethered to the membrane of EV derived from (e.g., originated from) a stromal cell. In a particular embodiment, TGF-β is tethered to the membrane of EV derived from a mesenchymal stromal cell (“MSC”). In an embodiment, the TGF-β isoform tethered to the EV membrane is TGF-β1. In an embodiment, the TGF-β is the latent form of TGF-β or an isoform thereof. In a particular embodiment, the TGF-β is the latent form of TGF-β1 or TGF-β3.

TGF-β acts as a master regulator protein in cell biology as it affects cell proliferation, survival, differentiation and numerous specialized activities. In accordance with the present invention, cells, particularly, mesenchymal stromal cells (MSC), not only produce soluble TGF-β, they also shed extracellular vesicles (EV) having membrane-tethered TGF-β that is strongly immunomodulatory (e.g., TGF-β tethered to the membrane of EV can affect proliferation, survival and function of immune cells, e.g., T cells). TGF-β in latent and active forms is tethered by molecules (e.g., glycoproteins, β-glycans and heparins) to the membrane surface of such cells (e.g., cancer-associated cells, stromal stem cells, dendritic cells, tumor cells) and also to the membranes of the extracellular vesicles (EV) that are produced and shed by these cells.

In an aspect, the invention provides TGF-β tethered to the membrane of EV derived from a given cell, e.g., a mesenchymal stromal cell (MSC), a cancer associated cell (e.g., fibroblast-like cell, or cancer-associated fibroblast), stromal cell, stem cell, or dendritic cell, as a quantifiable biomarker for monitoring an aspect of a disease or condition in a subject, such as disease progression, regression, remission, reduction, elimination, and the like in a subject, such as a patient having a disease or condition, e.g., cancer, autoimmune disease, inflammatory disease, or transplant rejection.

In an aspect, the invention provides an extracellular vesicle (EV) isolated from a mesenchymal stem cell (MSC) isolated from any tissue or organ site (e.g., umbilical cord tissue, placental tissue, bone marrow, blood, fat) and having TGF-β protein tethered to the EV membrane (called an MSC-derived membrane-tethered TGF-β EV herein). In an embodiment, the MSC can be grown and expanded in culture as a source of MSC-derived membrane-tethered TGF-β EV, which can be isolated and used as a therapeutic as described herein. In an embodiment, the MSC-derived, TGF-β-tethered EV suppresses lymphocyte activity, e.g., T-cell proliferation, in activated or stimulated lymphocytes, e.g., T cells. In addition, TGF-β-tethered EV also alter a variety of immune effector cells, such as natural killer cells, dendritic cells, monocytes and macrophages, and B cells.

In an aspect, the invention provides extracellular vesicles (EV) having membrane-tethered TGF-β isolated, or isolated and purified, from a biological sample of a subject or from a culture medium via suitable isolation methods. Such isolated EV with membrane-tethered TGF-β provide an effective therapy for treating a disease in a subject in need, for example, an immunosuppressive disease, autoimmune disease, transplant rejection, or cancer. In an embodiment, EV with membrane-tethered TGF-β selected and isolated by the methods described herein offer a more effective therapy than unselected EV in the treatment of disease in subjects in need. In an embodiment, the isolated EV with membrane-tethered TGF-β, derived from a biological fluid, cell or tissue source, provide an effective diagnostic biomarker, e.g., for detecting immunosuppression or lack thereof in a subject with disease.

In an aspect, methods are provided in which levels of TGF-β tethered to the membrane of EV from MSC or other cell types are quantified relative to a suitable control. The described EV comprising membrane-tethered TGF-β are also provided as biomarkers that are useful in methods of assessment and/or evaluation of the safety, activity, efficacy, or medical and clinical effectiveness of treatment, therapy, or an intervention in a patient or patient population, e.g., in a clinical trial setting. In an embodiment, EV having tethered TGF-β on the membrane are circulating EV, namely, those are isolatable in a biological or biofluid, cell, or tissue sample, e.g., blood, serum, plasma, urine, sputum, saliva, tears, cerebrospinal fluid and the like. In an embodiment, the EV circulate systemically in the bloodstream and/or the tissues.

In another aspect, the invention provides a method of quantifying TGF-β tethered to the membrane of EV (i.e., quantifying EV having membrane-tethered TGF-β) as a detectable and quantifiable biomarker of after treatment or therapy in a subject, for example, following oncology treatment or therapy or autoimmune disease, inflammatory disease, transplant rejection, or cardiac disease treatment or therapy. The method involves measuring (using a suitable quantification method) the level or amount of TGF-β tethered to the EV membrane in a sample obtained from a subject having active disease, at a predetermined time following treatment relative to the level or amount of a control. By way of example, a control can be the level or amount of TGF-β tethered to the EV membrane in a sample obtained from a healthy subject, or a subject having no disease or undetectable disease (a “normal” subject). In an embodiment of the method, the level of membrane-tethered TGF-β EV correlates with the level of immunosuppression of disease in a subject following treatment or therapy for the disease. In an embodiment, a higher level of membrane-tethered TGF-β on extracellular vesicles as quantified by the described methods is associated with a higher level of immunosuppression of disease treatment following the use of such EV with membrane-tethered TGF-β as a therapeutic for treatment of the disease. In an embodiment, the EV comprising membrane-tethered TGF-β are derived from mesenchymal stromal cells (MSC), e.g., either grown in culture or immortalized MSC.

In various embodiments of the above-aspects or any other aspect of the invention delineated herein, the TGF-β is tethered (i.e., membrane bound) to the membrane of EV derived from MSC or other cell types via one or more glycoproteins or types of glycoproteins, e.g., beta-glycans or heparin. In various embodiments of the above-aspects the TGF-β is an isoform of TGF-β, e.g., TGF-β1, TGF-β2, TGF-β3, or TGF-β4. In a particular embodiment, TGF-β is TGF-β1.

In various embodiments of the above-aspects, the membrane-tethered TGF-β EV contains a heterologous polypeptide or polynucleotide. In an embodiment, the polypeptide is a recombinant polypeptide heterologously expressed in the EV having membrane-tethered TGF-β or is loaded into the cell, e.g., an MSC producing the EV having membrane-tethered TGF-β, or extracellular vesicle ex vivo or in vitro. In an embodiment, the polynucleotide is a recombinant polynucleotide that is heterologously expressed in the EV having membrane-tethered TGF-β or is loaded into the cell, e.g., an MSC producing the EV having membrane-tethered TGF-β, ex vivo or in vitro.

In an embodiment of the above-aspects, TGF-β is tethered to the membrane of an EV derived from a cancer associated fibroblast which is a stromal cell. In an embodiment of the above-aspects, the stromal cell is derived from a tumor microenvironment. In various embodiments of the above-aspects, the tumor is a breast cancer tumor, pancreatic cancer tumor, brain cancer tumor, glioblastoma, melanoma, lung cancer tumor, ovarian cancer tumor, cervical cancer tumor, prostate cancer tumor, head and neck cancer tumor, or any other type of cancer or tumor. In a particular embodiment of the above-aspects, TGF-β is tethered to the membrane of an EV derived from a mesenchymal stromal cell (MSC) which is derived from any tissue, organ, or site. In various embodiments of the above-aspects, the EV comprising membrane-tethered TGF-β are isolated from a bodily fluid selected from the group consisting of blood, plasma, serum, saliva, sputum, urine, stool, semen, cerebrospinal fluid, prostate fluid, lymphatic drainage, bile fluid, peritoneal fluid and pancreatic secretions. In various embodiments of the above-aspects, the EV comprising tethered TGF-β are isolated from cell culture media. In various embodiments of the above-aspects, the EV comprising tethered TGF-β are isolated from cells cultured in conditioned medium obtained from a culture containing cancer cells, a culture containing cancer-associated cells, or from a culture containing MSC. In various embodiments of the above-aspects, the EV comprising tethered TGF-β derived from MSC also express CD105+, CD73+, CD90+ but have a CD45−, CD34− CD14−, CD19−, CD3−, HLA DR− biomarker phenotype. In an embodiment of the above aspects, the EV comprising tethered TGF-β is isolated from mammalian cells. In various embodiments of the above aspects, the EV comprising tethered TGF-β is an exosome, microvesicle, oncosome, or any extracellular vesicle type as described herein. In an embodiment of the above aspects, the TGF-β isoform is TGF-β1.

In another aspect, the invention provides a method for obtaining EV having membrane-tethered TGF-β from a mesenchymal stromal cell (MSC), in which the method involves isolating the tethered TGF-β EV from a biological sample of a subject or from a cell culture of MSC. In an embodiment, the amount of TGF-β that is tethered to the isolated EV is quantified.

In another aspect, the invention provides a method for obtaining EV having membrane-tethered TGF-β from a mesenchymal stromal cell (MSC) culture, in which the method involves culturing the MSC, or a cell or tissue source, such as umbilical cord cells, placental cells or tissue comprising MSC, in cell culture or conditioned medium, in which the MSC produce and secrete the membrane-tethered TGF-β EV, which can be isolated from the cell culture supernatant. In an embodiment, the amount of TGF-β that is tethered to the isolated EV is quantified as a measure of EV activity or potency. In embodiments, the amount or level of EV having membrane-tethered TGF-β produced by MSC grown in cell culture can be quantified. A high amount or level of membrane-tethered TGF-β may be indicative of the therapeutic potential of the membrane-tethered TGF-β EV, e.g., as an immunosuppressive therapeutic, in a subject in need following administration to the subject.

In another aspect, the invention provides an extracellular vesicle (EV) having TGF-β tethered to the membrane produced according to the method of the above aspects.

In another aspect, the invention provides a composition comprising a plurality of extracellular vesicles (EV), where the membrane of each EV contains tethered TGF-β.

In another aspect, the invention provides a composition for imaging analysis, for example, for use in assessing or monitoring disease status or effectiveness of disease treatment or therapy. Accordingly, the composition comprises an extracellular vesicle (EV) having membrane-tethered TGF-β isolated from a cell source, e.g., a stromal cell, a stromal-stem cells, a mesenchymal stromal cell, a cancer associated fibroblast (CAF), or a fibroblast-like cell, where the membrane-tethered TGF-β EV contains a detectable agent. In an embodiment, the detectable agent is an imaging agent. In other embodiments, the imaging agent is a nanoparticle, magnetite, nanoparticle, paramagnetic particle, microsphere, or nanosphere, which is selectively targeted to certain cells, e.g., cancer cells or disease-associated cells. In accordance with this embodiment, the membrane-tethered TGF-β EV comprising an imaging agent or label, for example, can be administered to a subject, e.g., as a theranostic, and can target cells and tissues in the body which express TGF-β receptors (TGFβR), such as sites of inflammation, tumor sites and microenvironments, etc.

In other aspects, the invention provides an isolated extracellular vesicle (EV) comprising TGF-β or an isoform thereof (e.g., TGF-β1, TGF-β2, TGF-β3, or TGF-β4) tethered to the membrane surface, wherein the EV is produced by an immortalized cell. In an embodiment, the immortalized cell is an immune privileged cell derived, for example, from umbilical cord, placenta, fetus, testes, or articular cartilage. In an embodiment, the immortalized cell is derived from a stromal cell, stem cell, stromal stem cell, mesenchymal stromal cell (MSC), cancer-associated cell, or fibroblast-like cell. In an embodiment, the extracellular vesicle (EV) is derived from a mesenchymal stem cell (MSC) and comprises recombinant TGF-β or a TGF-β isoform tethered to the membrane surface. In an embodiment, the TGF-β or isoform thereof is tethered to the membrane of the EV via attachment to one or more of a glycoprotein, β-glycan, or heparin. In an embodiment, the extracellular vesicle (EV) comprising TGF-β or an isoform, or an active or latent form thereof, tethered to the membrane surface is synthetically produced. In an embodiment, the extracellular vesicle (EV) having tethered TGF-β comprises at least one other tethered immunomodulatory molecule. In an embodiment, the at least one other tethered immunomodulatory molecule tethered to the EV in addition to TGF-β is selected from PD-1, PD-LI, B7-H4, B7-H5, CTLA-4, 4-1-BB, CD4, CD8, CD14, CD25, CD27, CD40, CD68, CD163, GITR, LAG-3, OX40, TIM3, TIM4, CEA, TLR, TLR2, etc., or cytokines, e.g., IL-4, IL-6, IL-7, IL-10, IL-12, IL-15, IL-17, IFN-γ, Flt3, BLys, chemokines, e.g., CCL21, or Galectin-1.

In an embodiment, the above-described extracellular vesicle (EV) having membrane-tethered TGF-β comprises an exogenous agent, such as a polypeptide, polynucleotide, or small molecule (e.g., a lipid or other hydrophobic small molecule, or a small molecule such as doxorubicin, cisplatin, or phosphatidyl ethanolamine). In an embodiment, the EV having membrane-tethered TGF-β comprises an exogenous agent, e.g., a recombinant polypeptide or polynucleotide that is heterologously expressed in a mesenchymal stromal cell (MSC) or loaded into a MSC or the EV ex vivo. In an embodiment, the polypeptide heterologously expressed by the EV as described herein is an antibody, a polypeptide that localizes to a specific cell type, or a therapeutic protein. In an embodiment, the EV as described herein comprises an exogenous agent that can be used for imaging purposes, for example, a nanoparticle, paramagnetic particle, microsphere, or nanosphere for magnetic imaging. In an embodiment, the extracellular vesicle (EV) as described herein is isolated from a biological or bodily fluid selected from blood, plasma, serum, saliva, sputum, urine, stool, semen, cerebrospinal fluid, prostate fluid, lymphatic drainage, bile fluid, peritoneal fluid, or pancreatic secretions. In an embodiment, the EV as described herein is isolated from cell culture medium, from cells cultured in conditioned medium, or from cells cultured in conditioned medium obtained from a culture comprising cancer cells. In an embodiment, the EV as described herein is isolated from a culture comprising a cancer-associated cell derived from a stromal cell, stem cell, fibroblast-like cell, stellate cell, or myofibroblast. In an embodiment of any of the above aspects, the transforming growth factor-beta (TGF-β) tethered to the membrane surface is an active or latent form of TGF-β or an isoform thereof.

In an aspect, the invention provides a mesenchymal stromal cell (MSC) which produces the extracellular vesicle (EV) having TGF-β tethered to the membrane as described hereinabove. In embodiments of any of the foregoing aspects, the EV as described herein is isolated from cells, tissue, or body fluid from a mammal or from a human patient. In embodiments of any of the foregoing aspects, the EV as described herein is an exosome or a microvesicle.

In another of its aspects, the invention provides a method of isolating mesenchymal stromal cell (MSC)-derived extracellular vesicles (EV) comprising membrane-tethered TGF-β (MSC-derived, membrane-tethered TGF-β EV), in which the method comprises culturing MSC, or a cell or tissue source of MSC, in cell culture or conditioned medium; and isolating the MSC-derived, membrane-tethered TGF-β EV from the cell culture or conditioned medium; and optionally, quantifying the amount of MSC-derived, membrane-tethered TGF-β EV from the cell or tissue source. In an embodiment of the method, the tissue source is selected from a biological fluid, umbilical cord tissue, placental tissue, fat, or bone marrow. In another embodiment of the method, the MSC are cultured in culture medium for from about 1 day to about 20 days. In another embodiment of the method, the culture or conditioned medium is a serum free chemically defined buffered medium or medium comprised of autologous serum and defined constituents. In another embodiment of the method, the MSC-derived, membrane-tethered TGF-β EV are isolated by one or more of affinity column chromatography, immune affinity capture, tangential flow filtration, precipitation, differential ultracentrifugation, density gradient centrifugation, or size exclusion chromatography as described herein. In another embodiment of the method, the amount or level of isolated EV having membrane tethered TGF-β, (e.g., the amount or level of TGF-β tethered to the membrane of EV), is quantified (measured) as described herein, for example, by a technique such as single vesicle nanoparticle tracking assay, vesiculometry, interferometry, or flow cytometry as known in the art and as described herein.

In another of its aspects, the invention provides a method of isolating mesenchymal stromal cell (MSC)-derived extracellular vesicles (EV) comprising membrane-tethered TGF-β (MSC-derived, membrane-tethered TGF-β EV) from a biological sample or cell culture medium, in which the method comprises: contacting the biological sample or cell culture medium containing or suspected of containing MSC-derived, membrane-tethered TGF-β EV with a substrate having attached thereto anti-TGF-β antibody under conditions sufficient to allow binding the TGF-β tethered to the membrane of the EV to the anti-TGF-β antibody; and isolating the MSC-derived, membrane-tethered TGF-β EV bound to the attached antibody. In an embodiment of the method, the membrane-tethered TGF-β EV are contacted with the substrate with attached antibody for at least 30 minutes. In an embodiment of the method, the membrane-tethered TGF-β EV are isolated from the substrate by elution with a buffer solution having a salt concentration, pH, pI, or ionic strength suitable for disrupting the binding interaction between the membrane-tethered TGF-β EV and the antibody. In another embodiment of the method, the amount (or level) of membrane-tethered TGF-β EV bound to the antibody by light interference measurement is quantified as described herein. In an embodiment, extracellular vesicles (EV) having membrane-tethered TGF-β are derived from mesenchymal stromal cells (MSC-derived, membrane-tethered TGF-β EV). In another embodiment of the method, the biological activity of the isolated membrane-tethered TGF-β EV, e.g., isolated MSC-derived, membrane-tethered TGF-β EV, is assayed, for example, by measuring the level of (i) suppression of mitogen-stimulated T cell proliferation; (ii) suppression of T cell cytokine production (iii) suppression of CD3/CD28-induced T cell proliferation; (iv) suppression of T cell production of IFNγ or IL-17; (v) suppression of CD69 expression by activated T cells; (vi) suppression of differentiation or expansion of a T regulator cell subset; (vii) suppression of natural killer (NK) cell differentiation or activation; (viii) suppression of maturation of dendritic cells (e.g., CD1a, MHCII, CD80, CD86 expression), or (ix) expansion of CD4+ or CD8+ T regulatory subsets or polarized (M2) anti-inflammatory macrophages by the isolated membrane-tethered TGF-β EV, e.g., the isolated MSC-derived, membrane-tethered TGF-β EV, using methods practiced by one skilled in the art.

In yet another of its aspects, the invention provides a method of diagnosing an autoimmune or inflammation related disease, condition, or disorder in a subject undergoing testing therefor, in which the method comprises obtaining a biological sample from the subject undergoing testing; detecting whether extracellular vesicles (EV) having TGF-β tethered to the membrane (membrane-tethered TGF-β EV) are present in the sample by quantifying the level of, membrane tethered-TGF-β EV relative to a control level; and diagnosing an autoimmune or inflammation related disease, condition, or disorder in the subject if the amount of membrane tethered-TGF-β EV is low or decreased relative to the control. In an embodiment of the method, the autoimmune related disease, condition, or disorder associated with a low or decreased membrane tethered-TGF-β EV is selected from one or more of asthma, atopic dermatitis, inflammatory bowel disease, psoriasis, multiple sclerosis, rheumatoid arthritis, type 1 diabetes, graft versus host disease, sarcoidosis, Sjogren's Disease, or lupus. In an embodiment, the method further comprises treating the subject diagnosed with the autoimmune disease, condition, or disorder by administering to the subject an effective amount of MSC-derived, membrane tethered-TGF-β EV to provide an immunosuppressive effect. In an embodiment of the method, the MSC-derived, membrane tethered-TGF-β EV are administered to the subject by a delivery route selected from intravenous, intra-spinal, intra-coronary, intra-lesional, or topical.

In another aspect, the quantity and isoform of EV expressing tethered-TGF-β present in biofluids originating from specific cell types (e.g., cancer cells, liver cells, bone marrow cells, brain, MSC) can be determined or identified based on the detection of surface markers. Because EV expressing membrane tethered-TGF-β can arise from any cell in the body, the specific cell type (or cell, organ, or tissue source) from which the EV with membrane tethered-TGF-β arises can be determined by isolating EV having specific cell surface markers (e.g. using immune affinity, magnetic beads, or other techniques) and quantifying the amount or level of membrane tethered-TGF-β that is on the membrane surface of the isolated cell-specific EV. Accordingly, in an aspect, the invention provides methods for determining the source or identity of the cell type that produces EV having membrane tethered TGF-β and the amount or level of TGF-β that is tethered to the membrane of the cell type in the circulation, e.g., a cancer cell, from which the EV having membrane tethered TGF-β are derived.

In another of its aspects, the invention provides a method of treating immunosuppression in a subject having a disease or condition, in which the method comprises: administering to the subject an effective amount of a treatment agent or regimen to reduce the level of mesenchymal stromal cell (MSC)-derived extracellular vesicles (EV) having TGF-β tethered to the membrane (MSC-derived membrane-tethered TGF-β EV present in the subject; wherein the reduced level of the MSC-derived, membrane tethered-TGF-β EV in the subject treats the immunosuppression ; and optionally, monitoring the subject at regular intervals to determine if the level of membrane tethered-TGF-β EV present in a biological sample from the subject has increased following said administration, wherein an increase in the level or amount of membrane tethered-TGF-β EV in the subject's biological sample is indicative of increased or rising levels of cells which produce the membrane tethered-TGF-β EV. In an embodiment of the above method, the disease or conditions is selected from cancer, infection, drug effects, stress, trauma, or degenerative disorder. In an embodiment, of the above method, the amount or level of membrane tethered-TGF-β EV present in the subject is reduced by administration of a treatment agent or regimen selected from one or more of neutralizing anti-TGF-β antibodies, antisense polynucleotides specific for TGF-β, or a pharmacological inhibitor of TGF-β.

In another of its aspects, the invention provides a method of diagnosing poor prognosis or increased risk for metastasis or malignancy in a subject with cancer, in which the method comprises: obtaining, at a first time point, a biological sample from a subject with cancer; quantifying the amount of extracellular vesicles (EV) having membrane-tethered TGF-β (membrane tethered-TGF-β EV) present in the subject's biological sample relative to a control amount at the first time point by a suitable procedure, for example, one or more of antibody immune capture, interferometry, flow cytometry, or nanoparticle tracking analysis fluorescence; obtaining, at a second time point following treatment of the subject with an anti-cancer therapeutic agent or regimen, a biological sample from the subject; quantifying the amount of membrane tethered-TGF-β EV present in the subject's biological sample relative to a control amount at the second time point (e.g., by one or more of antibody immune capture, interferometry, flow cytometry, or nanoparticle tracking analysis fluorescence); and diagnosing poor prognosis or increased risk for metastasis or malignancy in the subject with cancer when an increased or rising amount of the membrane tethered-TGF-β EV is present in the subject's biological sample at the first time point versus the second time point. In an embodiment of the method, the increased or rising amount of the membrane tethered-TGF-β EV present in the subject's biological sample at the first time point versus the second time point further indicates: recurrence of the primary cell source of the membrane tethered-TGF-β EV in the subject; and/or residual cancer burden, cancer recurrence, failure of cancer therapy, or resistance to therapy in the subject. In an embodiment of the method, a greater than or equal to an approximately 1.5-fold difference between the amount of membrane tethered-TGF-β EV in the subject's biological sample relative to the control amount indicates a low or reduced amount of membrane tethered-TGF-β EV in the subject. In other embodiments, a greater than or equal to an approximately 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13,5, 14, 14.5, 15, or greater-fold difference between the amount of membrane tethered-TGF-β EV in the subject's biological sample relative to the control amount indicates a low or reduced amount of membrane tethered-TGF-β EV in the subject. In an embodiment of the method, a greater than or equal to 1.5-fold difference between the amount of membrane tethered-TGF-β EV in the subject's biological sample relative to the control amount indicates an increased or rising amount of membrane tethered-TGF-β EV in the subject. In other embodiments, a greater than or equal to an approximately 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13,5, 14, 14.5, 15, or greater-fold difference between the amount of membrane tethered-TGF-β EV in the subject's biological sample relative to the control amount indicates an increased or rising amount of membrane tethered-TGF-β EV in the subject. In an embodiment of the method, the subject has a cancer selected from liver cancer, pancreatic cancer, prostate cancer, breast cancer, hepatocellular carcinoma, colon cancer, lung cancer, lymphoma, leukemia, melanoma, basal cell cancer, cervical cancer, colorectal cancer, stomach cancer, bladder cancer, anal cancer, bone cancer, brain tumor, esophageal cancer, gall bladder cancer, gastric cancer, testicular cancer, Hodgkin's Lymphoma, intraocular melanoma, kidney cancer, oral cancer, melanoma, neuroblastoma, Non-Hodgkin's Lymphoma, ovarian cancer, retinoblastoma, skin cancer, bucal cancer, throat cancer, thyroid cancer, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia, polycythemia vera, lymphoma, Hodgkin's disease, non-Hodgkin's disease, Waldenstrom's macroglobulinemia, heavy chain disease, sarcomas, carcinomas. fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, cholangiocarcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, or retinoblastoma.

In an embodiment of any of the above aspects, the biological sample is selected from blood, plasma, serum, saliva, sputum, urine, stool, semen, cerebrospinal fluid, prostate fluid, lymphatic drainage, bile fluid, peritoneal fluid, pancreatic secretions, cells, or tissue. In an embodiment of the methods of any of the above aspects, a greater than or equal to an approximately 1.5-fold difference between the amount of membrane tethered-TGF-β EV in the subject's biological sample relative to the control amount indicates a low or reduced amount of membrane tethered-TGF-β EV in the subject. In an embodiment of the methods of any of the above aspects, a greater than or equal to an approximately 1.5-fold difference between the amount of membrane tethered-TGF-β EV in the subject's biological sample relative to the control amount indicates an increased or rising amount of membrane tethered-TGF-β EV in the subject. In an embodiment of the methods of any of the above aspects, the control comprises one or more of the quantity of total sample protein, nucleic acid or lipid; the quantity of EV-specific proteins (e.g., one or more of CD9, CD63, CD81, Tsg101, flotillin, synectin, LAMP-2, or Alix); or total number of EV in the subject's sample. In an embodiment of the methods of any of the above aspects, the control comprises a healthy subject who has normal quantities of membrane tethered-TGF-β EV, or a subject who is disease free, or a subject who was sampled prior to disease.

In another of its aspects, the invention provides a method of obtaining an immunosuppressive therapeutic for treating a subject in need thereof; in which the method comprises: culturing a cell or tissue source of mesenchymal stromal cells (MSC) in cell culture or conditioned medium for a time sufficient for secretion of extracellular vesicles (EV) having membrane-tethered TGF-β by the MSC (MSC-derived, membrane-tethered TGF-β EV) and under conditions to increase levels of membrane-tethered TGF-β EV produced by the MSC; quantifying the MSC-derived, membrane-tethered TGF-β EV produced by the cultured MSC relative to a control; isolating MSC-derived, membrane-tethered TGF-β EV from the cell culture or conditioned medium for use as an immunosuppressive therapeutic in a subject having a disease requiring suppression of immune cells; and optionally, administering or recommending the administration of the isolated MSC-derived, membrane-tethered TGF-β EV to a subject having a disease selected from inflammation, autoimmunity, or transplant rejection and needing an immunosuppressive therapeutic. In an embodiment of the method, the MSC are subjected to hypoxia and immune mediator molecules to increase the levels of MSC-derived, membrane-tethered TGF-β EV in the culture by approximately 1.5-fold to 10-fold. In an embodiment of the method, the MSC-derived, membrane-tethered TGF-β EV are quantified by single vesicle nanoparticle tracking assay (NTA), vesiculometry, interferometry, or flow cytometry. In an embodiment of the method, the MSC-derived, membrane-tethered TGF-β EV are isolated from the culture medium by one or more of affinity column chromatography, immune affinity capture, tangential flow filtration, precipitation, differential ultracentrifugation, density gradient centrifugation, or size exclusion chromatography.

In an aspect, the invention provides an in vitro method of enhancing production of mesenchymal stromal cell (MSC)-derived extracellular vesicles (EV) comprising membrane-tethered TGF-β (MSC-derived, membrane-tethered TGF-β EV), in which the method comprises: culturing MSC in conditioned medium comprising an effect amount of one or more immune mediator molecules selected from the group consisting of interferon-gamma (IFNγ), tumor necrosis factor (TNF), lipopolysaccharide (LPS) and interleukin-17 (IL-17) for a time sufficient for the MSC to produce an enhanced amount of MSC-derived, membrane-tethered TGF-β EV, wherein the MSC are immortalized or native MSC. In an embodiment, of the method, the MSC are exposed to hypoxic conditions prior to culturing with the immune mediator molecules. In an embodiment of the method, the hypoxic conditions comprise growth in 1% O₂ for about 24 hours. In an embodiment of the method, the MSC-derived, membrane-tethered TGF-β EV are cultured for about 24 hours to about 20 days. In an embodiment of the method, the one or more immune mediator molecules are present in the culture in an amount of from 5-50 ng/ml. In an embodiment of the method, the amount of MSC-derived, membrane-tethered TGF-β EV produced by the MSC is enhanced approximately 1.5-fold to 10-fold. In other embodiments, the amount of MSC-derived, membrane-tethered TGF-β EV in the culture is increased by at least about or equal to 1.5-fold to 25-fold, or by at least about or equal to 1.5-fold to 15-fold, or by at least about or equal to 1.5-fold to 10-fold, or by at least about or equal to 1.5-fold to 5-fold, including values therebetween. In an embodiment, the method comprises isolating the MSC-derived, membrane-tethered TGF-β EV from the culture medium.

In an aspect, the invention provides a method of modulating the function of an immune cell involved in an immune response against disease in a subject, in which the method comprises contacting the cell with the mesenchymal stromal cell (MSC)-derived extracellular vesicles (EV) having membrane-tethered TGF-β (MSC-derived, membrane-tethered TGF-β EV) or the pharmaceutical composition as described in any of the above aspects and herein. In embodiments, the contact between MSC-derived, membrane-tethered TGF-β EV and immune cell can occur in vitro, in vivo, ex vitro. In an embodiment, immune cells of a patient can be isolated and exposed to MSC-derived, membrane-tethered TGF-β EV ex vivo or in vitro for a predetermined time period. Thereafter, the patient's cells can be introduced, e.g., via injection or transplant, back into the patient where they can exert an immunosuppressive effect on the body.

In another of its aspects, the invention provides a method of treating immunosuppression induced or caused by a disease or condition selected from cancer, infection, drugs, stress, trauma, degenerative disorder in a subject in need thereof, in which the method comprises: administering to the subject an effective amount of a treatment agent or regimen to reduce the amount or level of extracellular vesicles (EV) having TGF-β tethered to the membrane present in the subject. In an embodiment, a reduced amount or level of membrane tethered-TGF-β EV in the subject as quantified in a biological sample of a subject by the methods described herein indicates treatment of the immunosuppression induced or caused by the disease or condition selected from cancer, infection, drugs, stress, trauma, degenerative disorder. In an embodiment, the method optionally includes monitoring the subject at regular intervals to determine if the level or amount of_{T} membrane tethered-TGF-β EV present in a biological sample from the subject has increased following said administration of the treatment agent or regimen that reduces the level of membrane-tethered TGF-β EV, wherein an increase in the level or amount of membrane tethered-TGF-β EV in the subject's biological sample is indicative of increased or rising levels of cells, e.g., MSC which produce the membrane tethered-TGF-β EV. In an embodiment of the method, the amount or level of membrane tethered-TGF-β EV present in the subject is reduced by administration of a treatment agent or regimen selected from one or more of neutralizing anti-TGF-β antibodies, heparinases or other enzymes, for example, delivered systemically or targeted to specific subsets of EV, that alter tethered-TGF-β activity; antisense polynucleotides specific for TGF-β; or a pharmacological inhibitor of TGF-β

In another aspect, the invention provides a method of obtaining an immunosuppressive therapeutic for treating a subject in need thereof; in which the method comprises: culturing a cell or tissue source of mesenchymal stromal cells (MSC) in cell culture or conditioned medium for a time sufficient for secretion of extracellular vesicles (EV) having membrane-tethered TGF-β by the MSC (MSC-derived, membrane-tethered TGF-β EV) and under conditions to increase levels of membrane-tethered TGF-β EV produced by the MSC; quantifying the MSC-derived, membrane-tethered TGF-β EV produced by the cultured MSC relative to a control; obtaining isolated MSC-derived, membrane-tethered TGF-β EV from the cell culture or conditioned medium for use as an immunosuppressive therapeutic in a subject having a disease requiring suppression of immune cells; and optionally, administering or recommending the administration of the isolated MSC-derived, membrane-tethered TGF-β EV to a subject having a disease selected from inflammation, autoimmunity, or transplant rejection and needing an immunosuppressive therapeutic. In an embodiment, MSC are subjected to hypoxia and immune mediator molecules to increase the amount of MSC-derived, membrane-tethered TGF-β EV in the culture by about or equal to 1.5-fold to 4 fold. In other embodiments, the amount of MSC-derived, membrane-tethered TGF-β EV in the culture is increased by about or equal to 1.5-fold to 25-fold, or by about or equal to 1.5-fold to 15-fold, or by about or equal to 1.5-fold to 10-fold, or by about or equal to 1.5-fold to 5-fold, including values therebetween. In an embodiment, the levels of MSC-derived, membrane-tethered TGF-β EV produced by the MSC is enhanced by 1.5-4 fold. In embodiments of the method, the MSC-derived, membrane-tethered TGF-β EV are isolated from the culture medium by one or more of affinity column chromatography, immune affinity capture, tangential flow filtration, precipitation, differential ultracentrifugation, density gradient centrifugation, or size exclusion chromatography.

In another aspect, the invention provides a MSC-derived, membrane-tethered TGF-β EV produced according to the methods of any one of the above aspects.

In another aspect, the invention provides a pharmaceutical composition comprising the mesenchymal stromal cell (MSC)-derived extracellular vesicles (EV) having membrane-tethered TGF-β (MSC-derived, membrane-tethered TGF-β EV) according to the above aspects.

In an embodiment of the above aspects, the MSC-derived, membrane-tethered TGF-β EV cause suppression of an autoimmune disease, an inflammatory disease, or transplant rejection, or inhibition of tumor cell proliferation in the subject.

In another of its aspects, the invention provides a method of detecting a biomarker of immunosuppression associated with active disease in a subject, in which the method comprises: obtaining a biological sample from the subject; assaying for the presence of extracellular vesicles (EV) having membrane-tethered TGF-β (membrane tethered-TGF-β EV), e.g., derived from any cell type or from specific cell types present in the sample, as a biomarker of active disease associated immunosuppression in the sample; assaying the sample for the presence of extracellular vesicles (EV) having membrane-tethered TGF-β as a biomarker of immunosuppression; quantifying the amount or level of membrane tethered-TGF-β EV relative to a control amount or level, if said EV are present in the sample; and detecting immunosuppression associated with active disease in the subject, if the subject's sample contains an increased amount or level of the membrane-tethered TGF-β EV biomarker relative to the control. In embodiments of the method, an active disease is an autoimmune disease, an inflammatory disease, transplant rejection, or cancer. In an embodiment of the method, assaying for the presence of the membrane tethered-TGF-β EV biomarker comprises affinity column chromatography, immune affinity capture, tangential flow filtration, precipitation, differential ultracentrifugation, density gradient centrifugation, or size exclusion chromatography. In an embodiment of the method, quantifying the amount of the membrane tethered-TGF-β EV biomarker comprises one or more of single vesicle nanoparticle tracking assay, vesiculometry, interferometry, or flow cytometry. In an embodiment of the method, greater than or equal to an approximately 1.5-10-fold change in the level of the membrane tethered-TGF-β EV biomarker is indicative of biologically relevant immunosuppression in the subject. In other embodiments, a change in the amount of membrane-tethered TGF-β EV in the samples of about or equal to 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 12-fold, 15-fold, 20-fold, 25-fold, or greater, including values therebetween is indicative of biologically relevant immunosuppression in the subject.

In another of its aspects, the invention provides a method of treating reduced immunosuppression in a subject having a disease or condition improved by an immunosuppression therapy, in which the method comprises quantifying the amount of extracellular vesicles (EV) having membrane-tethered TGF-β in a biological sample obtained from a subject in need relative to a control amount; wherein (i) a reduced amount of the membrane-tethered TGF-β EV in the subject's sample or (ii) a reduced amount of the level of TGF-β tethered to the EV in the subject's sample relative to a control indicates reduced immunosuppression in the subject; and administering or recommending the administration of an effective amount of isolated MSC-derived membrane-tethered TGF-β EV to the subject, if the subject's sample contains a reduced amount of membrane-tethered TGF-β EV, to augment immunosuppression activity in the subject having a disease or condition improved by the immunosuppressive therapy. In an embodiment of the method, an increase in membrane-tethered TGF-β EV in the sample of greater than or equal to 1.5-fold relative to the control indicates an increased amount of immunosuppression. In other embodiments, a change in the amount of membrane-tethered TGF-β EV in the sample of about or equal to 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 12-fold, 15-fold, 20-fold, 25-fold, or greater, including values therebetween indicates an increased amount of immunosuppression. In an embodiment of the method, the disease or condition improved by immunosuppression therapy is selected from autoimmune disease (e.g., asthma, atopic dermatitis, inflammatory bowel disease, psoriasis, multiple sclerosis, rheumatoid arthritis, type 1 diabetes, graft versus host disease, sarcoidosis, Sjogren's Disease, or lupus), inflammatory disease, chronic inflammation, or allergy. In an embodiment of the method, MSC-derived membrane-tethered TGF-β EV are administered to the subject by a mode of administration selected from intravenous, subcutaneous, intra-spinal, intra-coronary, intra-lesional, or topical. In an embodiment of the method, the MSC-derived, membrane-tethered TGF-β are isolated by one or more of affinity column chromatography, immune affinity capture, tangential flow filtration, precipitation, differential ultracentrifugation, density gradient centrifugation, or size exclusion chromatography.

In yet another of its aspects, the invention provides a method of treating elevated or increased immunosuppression in a subject having cancer, in which the method comprises: quantifying the amount or level of extracellular vesicles (EV) having membrane-tethered TGF-β in a biological sample obtained from a subject having cancer relative to a control amount; wherein an increased amount of membrane-tethered TGF-β EV, e.g., derived from any cell type, or derived from a specific cell source (e.g., an identified subset of membrane-tethered TGF-β EV from a specific cell source), in the sample indicates elevated or increased immunosuppression in the subject; and administering or recommending the administration of an effective amount of a therapeutic agent or regimen to decrease or neutralize the amount of membrane-tethered TGF-β EV in the subject, if the subject's sample contains an increased amount of membrane-tethered TGF-β EV, so as to decrease the immunosuppression in the subject having cancer. In an embodiment of the method, elevated or increased immunosuppression in the subject is associated with one or more of chemotherapy, drug, or radiation treatment. In an embodiment of the method, the therapy to decrease or neutralize the amount of MSC-derived membrane-tethered TGF-β EV comprises an effective amount of anti-TGF-β neutralizing antibodies, anti-TGF-β oligonucleotides, RNAi, anti-sense sequences; TGF-β gene therapy, or pharmacological inhibitors of TGF-β. In an embodiment of the method, the therapeutic agent or regimen to decrease or neutralize the amount of membrane-tethered TGF-β EV comprises an effective amount of one or more of anti-TGF-β neutralizing antibodies, anti-TGF-β oligonucleotides, RNAi-specific for TGF-β, anti-sense sequences specific for TGF-β; TGF-β gene therapy, or pharmacological inhibitors of TGF-β. In an embodiment of the method, the therapy to decrease or neutralize the amount of membrane-tethered TGF-β EV further comprises reducing or eliminating the number of cells (e.g. cancer cells, cancer associated cells, fibroblast-like cells, immune cells) which produce membrane-tethered TGF-β EV, for example, byaphaeresis, targeted cytotoxicity, or chemotherapeutic agent treatment. In an embodiment of the method, the therapy to decrease or neutralize the amount of membrane tethered TGF-β EV is combined with anti-cancer and anti-tumor approaches that reduce tumor or cancer cell burden. In an embodiment of the method, membrane-tethered TGF-β EV are quantified by single vesicle nanoparticle tracking assay (NTA), vesiculometry, interferometry, or flow cytometry. In an embodiment of the method, the control comprises a healthy or normal subject who has normal levels of membrane tethered-TGF-β EV or a subject who is disease free or a subject who was sampled prior to disease.

In another of its aspects, the invention provides a method of producing a population of mesenchymal stromal cell (MSC)-derived extracellular vesicles having membrane-tethered TGF-β (MSC-derived, membrane-tethered TGF-β EV) which has reduced immunosuppression in a subject, in which the method comprises subjecting a biological sample or cell culture medium comprising MSC that produce extracellular vesicles (EV) having membrane-tethered TGF-β and extracellular vesicles (EV) not having or having a significantly reduced amount of membrane-tethered TGF-β to a procedure that produces a population of MSC-derived EV that do not have membrane tethered TGF-β or that have reduced amounts of membrane-tethered TGF-β; and isolating the MSC-derived EV that do not have membrane-tethered TGF-β or that have a reduced amount of membrane-tethered TGF-β, thereby producing a population of MSC-derived EV having reduced immunosuppression when administered to a subject. In an embodiment of the method, the isolating step comprises contacting the biological sample or cell culture medium to an immune affinity substrate having immobilized thereon an anti-TGF-β binding agent that binds to the TGF-β tethered to the membrane of the EV, thereby retaining the MSC-derived EV having membrane-tethered TGF-β on the substrate and separating the MSC-derived EV having membrane-tethered TGF-β from the MSC-derived EV not having membrane-tethered TGF-β. In an embodiment of the method, before the isolating step, the MSC-derived extracellular vesicles (EV) are treated with an effective amount of an enzyme selected from a proteinase, a glycanase, or a heparinase to remove the TGF-β tethered to the membrane of the EV, thereby producing a population of MSC-derived EV having reduced immunosuppression when administered to a subject. In an embodiment, the population of MSC-derived EV having reduced immunosuppression retains other beneficial biological activities or show improved safety.

In an embodiment of any of the above aspects, the subject is a mammal or a human patient. In embodiments of any of the above aspects, the EV comprising membrane-tethered TGF-β are isolated from a biological fluid (body fluid) selected from the group consisting of blood, plasma, serum, saliva, sputum, urine, stool, semen, cerebrospinal fluid, prostate fluid, lymphatic drainage, bile fluid, peritoneal fluid and pancreatic secretions. In embodiments of any of the above aspects, the EV comprising membrane-tethered TGF-β is isolated from cell culture medium or from cells or tissue cultured in conditioned medium. In an embodiment of the methods of any of the above aspects, the control comprises one or more of the quantity of total sample protein, nucleic acid or lipid; the quantity of EV-specific proteins (e.g., one or more of CD9, CD63, CD81, Tsg101, flotillin, synectin, LAMP-2, or Alix); or total number of EV in the subject's sample. In another embodiment of the methods of any of the above aspects, the control comprises a healthy or normal subject who has a normal quantity of membrane tethered-TGF-β EV or a subject who is disease free or a subject who was sampled prior to disease. In embodiments of the methods of any of the above aspects, the membrane-tethered TGF-β EV are quantified by single vesicle nanoparticle tracking assay, vesiculometry, interferometry, or flow cytometry.

In another of its aspects, the invention provides a composition for imaging cells or tissue, the composition comprising an extracellular vesicle (EV) as described in the above aspects, in which the EV contain an imaging agent. In embodiments, the imaging agent is a nanoparticle, magnetite, nanoparticle, paramagnetic particle, microsphere, nanosphere, and is selectively targeted to cancer cells.

In another aspect, the invention provides a kit for providing to a subject an extracellular vesicle (EV) derived from mesenchymal stromal cells (MSC) and comprising membrane-tethered TGF-β or an isoform thereof (MSC-derived, membrane-tethered TGF-β EV) as a therapeutic agent, the kit comprising MSC-derived, membrane-tethered TGF-β EV isolated from MSC. In an embodiment, the MSC-derived, membrane-tethered TGF-β EV is an immunosuppressive agent.

In another aspect, the invention provides a kit for providing to a subject an extracellular vesicle (EV) derived from mesenchymal stromal cells (MSC) in which membrane-tethered TGF-β has been removed or substantially depleted from the EV as an agent to reduce inflammation or immunosuppression, the kit comprising MSC-derived EV wherein the membrane-tethered TGF-β is removed or substantially depleted.

In another aspect, the invention provides a kit for delivering a bioactive or imaging agent to a cell, the kit comprising an extracellular vesicle (EV) comprising membrane-tethered TGF-β or an isoform thereof isolated from a mesenchymal stromal cell (MSC), wherein the extracellular vesicle (EV) comprises the bioactive or imaging agent.

In the foregoing aspects, the kit further contains instructions for use and suitable containers for the components included therein.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide, polynucleotide, or fragment, or analog thereof, small molecule, or other biologically active molecule.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

As used herein, the term “animal” refers to any member of the animal kingdom. In an embodiment, the term “animal” refers a mammal. In an embodiment, the term “animal” refers to humans at any stage of development or any non-human animal at any stage of development. An animal may encompass various species of mammals, nonlimiting examples of which include non-human primates, dogs, cats, goats, pigs, rabbits, horses, camels, llamas, mice, rats, guinea pigs, gerbils, and the like. In other embodiments, an animal may encompass non-mammalian species, such as birds, fish, reptiles, and the like. In some embodiments, the term “animal” may refer to a transgenic or genetically engineered animal or a clone.

The term “antibody,” as used herein, e.g., an anti-TGF-β-specific antibody, refers to an immunoglobulin molecule which specifically binds with an antigen. Methods of preparing antibodies are well known to those of ordinary skill in the science of immunology. Antibodies can be intact immunoglobulins derived from natural sources or from recombinant sources and can be immunoreactive portions of intact immunoglobulins. Antibodies are typically tetramers of immunoglobulin molecules. Tetramers may be naturally occurring or reconstructed from single chain antibodies or antibody fragments. Antibodies also include dimers that may be naturally occurring or constructed from single chain antibodies or antibody fragments. The antibodies in the present invention may exist in a variety of forms including, for example, polyclonal antibodies, monoclonal antibodies, Fv, Fab and F(ab′) 2, as well as single chain antibodies (scFv), humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antibody specifically binds to a TGF-β polypeptide.

The term “antibody fragment” refers to a portion of an intact antibody and refers to the antigenic determining variable regions of an intact antibody. Examples of antibody fragments include, but are not limited to, Fab, Fab′, F(ab′)₂ and Fv fragments, linear antibodies, scFv antibodies, single-domain antibodies, such as camelid antibodies (Riechmann, 1999, J. Immunol. Methods, 231:25-38), composed of either a VL or a VH domain which exhibit sufficient affinity for the target, and multispecific antibodies formed from antibody fragments. Antibody fragments also include a human antibody or a humanized antibody or a portion of a human antibody or a humanized antibody.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

A “cancer associated fibroblast” (CAF) refers to a fibroblast cell that grows in proximity to cancer cells (e.g., in stroma) or in conditioned medium in which cancer cells had previously been cultured. Gene expression of CAFs is frequently altered following their growth in cancer conditioned media or in stroma. For example, CAFs exhibit increased expression of one or more marker proteins, including alpha-smooth muscle actin (a-SMA), PDGFRβ, collagen, vimentin (FSP-1), S100, metalloproteinases, NG2, SDF1 (CXCL12), CD34, fibroblast activation protein (FAP) and FSP-1 (as well as CD31), Thy-1, and gremlin. A CAF may express reduced levels of laminin relative to a reference. In addition to stromal cells, CAFs may be derived from cells having proximity to a tumor in vivo. Thus, CAFs may be derived from cells associated with blood vessels or local deposits of fat near a tumor. In some instances, a CAF or subtype thereof is identified at a site distant from the tumor via biomarker analysis. A “cancer associated cell” (CAC) is similar to a CAF. Nonlimiting examples of CACs include brain derived glia, oligodendroglia, microglia, and breast-EMT and bone marrow stem cells which have become CACs.

By “control” is meant a standard or reference condition. The term “control” refers to a standard against which results are compared. In some embodiments, a control is used at the same time as a test variable or subject to provide a comparison. In some embodiments, a control is a historical control that has been performed previously, a result or amount or level that has been previously known or obtained, or an otherwise existing record. A control may be a positive or negative control.

By “decreases” is meant a reduction by at least about 5% relative to a reference level. A decrease may be by 5%, 10%, 15%, 20%, 25% or 50%, or even by as much as 75%, 85%, 95% or more. Conversely, an “increase” refers to a gain, rise, augmentation, amplification, or growth of at least about 5% relative to a reference level. An increase may be by 5%, 10%, 15%, 20%, 25% or 50%, or even by as much as 75%, 85%, 95% or more.

By “an effective amount” is meant the amount of an agent required to ameliorate the symptoms of a disease relative to an untreated patient. In one embodiment, the disease is an autoimmune disease or condition (e.g., multiple sclerosis), an inflammation-related disease or condition, cardiac disease, or a transplant rejection condition such as graft versus host disease (GVHD). In another embodiment, the disease is cancer (e.g., breast cancer, colon cancer, lung cancer (e.g., small cell lung cancer), brain cancer, prostate cancer, bladder cancer, ovarian cancer, cervical cancer, testicular cancer, pancreatic cancer, renal cancer, head and neck cancer, stomach cancer, gall bladder cancer, melanoma, cholangiocarcinoma, hepatocellular carcinoma, hepatoma, and other cancers defined as neoplasias herein). In other embodiments, the disease is an autoimmune or inflammatory disorder or condition (e.g., asthma, allergy, coeliac disease, glomerulonephritis, hepatitis, inflammatory bowel disease, reperfusion injury and transplant rejection, atherosclerosis, periodontitis, arthritis, rheumatoid arthritis). In other embodiments, the disease is cardiac disease and related disorders (e.g., myocardial infarction, coronary syndrome). In other embodiments, the disease is a single gene disorder including, but not limited to, cystic fibrosis, sickle cell anemia, Tay-Sachs disease, myotonic dystrophy, Duchenne muscular dystrophy, Fragile X syndrome, glycogen storage diseases, and spinal muscular atrophy. As would be appreciated by one of ordinary skill in the art, the exact amount required to treat a disease will vary from subject to subject, depending on age, general condition of the subject, the severity of the condition being treated, the particular compound and/or composition administered, and the like. The effective amount of active agent(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “exogenous” is meant foreign or heterologous. An exogenous agent is one that is not naturally occurring in the cell, such as a protein that is recombinantly expressed.

As used herein, the term “extracellular vesicle (EV)” refers to a membrane (e.g., lipid bilayer)-containing vesicle released (secreted) to the extracellular environment by different cell types. Extracellular vesicles (EV) encompass a number of different membraned vesicles produced by cells, the names of which include, for example, microvesicles, epididimosomes, argosomes, exosome-like vesicles, microparticles, promininosomes, prostasomes, dexosomes, texosomes, archeosomes, oncosomes, and exersomesectosomes, microparticles and shedding microvesicles. Extracellular vesicles (EV) circulate through body fluids, including blood, plasma, serum and urine. Circulating EV may contain exosomes and microvesicles (MV).

An “exosome” refers to a small membrane extracellular vesicle of ˜30-300 nm or ˜40-120 nm diameter that is secreted from producing cells into the extracellular environment, as described initially by Trams, E. G. et al., 1981, Biochim. Biophys. Acta, 645(1):63-70. The surface (membrane surface) of an exosome comprises a lipid bilayer from the membrane of the donor cell, and the lumen of the exosome is topologically the same as the cytosol from the cell that produces the exosome. The exosome contains proteins, RNAs, lipids, and carbohydrates of the producing cell, though some may be modified or added to the exosome after its release from the cell, either through natural processes or by experimental manipulation. Illustrative exosome markers include Alix, Tsg101, tetraspanins (CD81, CD63, CD9), flotillin, synectin, or LAMP-2.

The term “microvesicle” (abbreviated “MV”) refers to a single membrane vesicle secreted by different cell types. MV may have a diameter (or largest dimension where the particle is not spheroid) of between about 10 nm to about 5000 nm (e.g., between about 50 nm and 1500 nm, between about 75 nm and 1500 nm, between about 75 nm and 1250 nm, between about 50 nm and 1250 nm, between about 30 nm and 1000 nm, between about 50 nm and 1000 nm, between about 100 nm and 1000 nm, between about 50 nm and 750 nm, etc.). Microvesicles originate from cells, yet different subpopulations of microvesicles may exhibit different surface/lipid characteristics. Typically, at least part of the membrane of the microvesicle is directly obtained from a cell (also known as a donor cell). Microvesicles may originate from cells by membrane inversion, exocytosis, shedding, blebbing, and/or budding. Depending on the manner of generation (e.g., membrane inversion, exocytosis, shedding, or budding), microvesicles may exhibit different surface/lipid characteristics. Illustrative microvesicle markers include integrins, selectins and CD40. Microvesicles have been called by alternative names in the art, such as, for example, EV, exosomes, membrane particles, exosome-like particles, and apoptotic vesicles.

By “fragment” is meant a portion (e.g., at least 10, 25, 50, 100, 125, 150, 200, 250, 300, 350, 400, or 500 amino acids or nucleic acids) of a protein or nucleic acid molecule that is substantially identical to a reference protein or nucleic acid and retains the biological activity of the reference.

By “heterologous” is meant originating in a different cell type or species from the recipient. Heterologous may be used interchangeably with exogenous herein.

A “host cell” is any prokaryotic or eukaryotic cell that contains either a cloning vector or an expression vector. This term also includes those prokaryotic or eukaryotic cells that have been genetically engineered to contain the cloned gene(s) in the chromosome or genome of the host cell.

By “inhibits a neoplasia or cancer” is meant decreases the propensity of a cell to develop into a neoplasia or cancer, or slows, decreases, or stabilizes the growth or proliferation of a neoplasia or cancer.

The term “immunomodulator” refers to an agent, e.g., protein, polypeptide, peptide, small molecule, that can modify (e.g., alter, suppress, augment) an immune response or the functioning or activity of the immune system or cells of the immune system (e.g., white blood cells such as T lymphocytes, B lymphocytes, macrophages, dendritic cells, natural killer cells (NK), etc.), for example, by inhibiting or suppressing immune cell function or activity, or by activating immune cell function, or stimulating antibody production. An immunomodulator can modify the normal or typical activity or function of an immune cell, e.g., by increasing or suppressing cell proliferation and/or differentiation, or by altering an immune cell's response to cytokines or external stimuli. In an embodiment, an immunomodulator can weaken or reduce or suppress the activity of the immune system, e.g., white blood cells of the immune system. In the case of autoimmune disease, e.g., arthritis or multiple sclerosis, inflammatory disease, transplant rejection, or heart disease, such an immunomodulatory activity is desirable to aid in the treatment or therapy of the disease. In the present case, the extracellular vesicles (EV) having membrane-tethered TGF-β can influence a subject's immune response, as membrane-tethered TGF-β on EV interacts potently with cells of the immune system (particularly, T cells such as CD4+ and CD8+ T cells and dendritic cells) to upregulate or downregulate specific aspects of the immune response. The enhancement or suppression of the immune response by EV having membrane-tethered TGF-β can depend on several other factors, e.g., amount (dose), timing, mode of administration, type of disease, influence of other molecules, such as cytokines, integrins, signaling molecules, cell surface receptors, etc., that direct and influence immune responses. (See, e.g., Worthington, J. J. et al., 2012, Immunobiology, Vol. 217:1259-1265). According to the invention, TGF-β tethered to the EV membrane plays an active role in the regulation of T cell development and function by promoting or suppressing different T cell subsets or other immune effector cells (e.g., monocytes, macrophages, natural killer (NK) cells, B cells, and dendritic cells) and tuning immune responses.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

As used herein, the term “isolated” refers to a substance, molecule, or entity that has been either separated from at least some of the components with which it was associated when initially produced in nature or through an experiment, and/or produced, prepared, or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components.

By “marker” is meant any protein or polynucleotide having an alteration in expression level or activity that is associated with a disease or disorder.

A “mesenchymal stromal cell” or “mesenchymal stromal stem cell” (MSC) refers to a spindle shaped cell that is isolated from bone marrow, adipose tissue, and other tissue sources, that adheres to plastic, and that has the capacity to differentiate into different cell types, e.g., osteoblasts, adipocytes, chondroblasts, in vitro. (Horwitz, E. M. et al., 2006, Curr. Opin. Hematol., 13(6):419-425). MSC, considered to be immunoprivileged (i.e., to escape recognition as “foreign” by immune cells), have immunomodulatory effects, i.e., they can effect immune effector cell (e.g., T cell) function. A general surface marker phenotype profile of MSCs comprises CD105+, CD73+, CD90+, CD45−, CD34− CD14−, CD19−, CD3−, HLA DR−. In vivo immunomodulatory effects of MSC include a reversal of the evolution of graft-versus-host disease (GVHD) (Friedenstein, A. J. et al., 1968, Transplantation, 6:230-247; Ringden, O. et al., 2006, Transplantation, 81:1390-1397; LeBlanc, K. et al., 2004, Lancet, 363:1439-1441) and the amelioration of the course of chronic progressive experimental autoimmune encephalomyelitis (EAE) in a mouse model of multiple sclerosis (Zappa, E. et al., 2005, Blood, 106:1755-1761). MSC also have been observed to integrate into the environment of solid tumors (Studeny, M. et al., 2006, J. Natl Cancer Inst., 96:1593-1603; Marini, F. et al., 2006, In: Stem Cell Transplantation, Eds. Ho, A. D. et al., Wiley-VCH Verlag, Weinheim, Germany, pp. 157-175). Accordingly, extracellular vesicles (EV) comprising membrane-tethered TGF-β derived or isolated from these cells are similarly immunoprivileged and may be used as cell therapy for immune modulation, tissue regeneration, and as delivery for bioactive agents, e.g., anti-tumor agents.

By “modification” is meant any biochemical or other synthetic alteration of a nucleotide, amino acid, or other agent relative to a naturally occurring reference agent.

By “neoplasia” is meant any disease that is caused by or results in inappropriately high levels of cell division, inappropriately low levels of apoptosis, or both. For example, cancer is a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, cholangiocarcinoma (also termed bile duct carcinoma), choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma. Lymphoproliferative disorders are also considered to be proliferative diseases.

In some embodiments, “cancer” can include histologic and molecular subtypes of liver cancer, pancreatic cancer, prostate cancer, breast cancer, hepatocellular carcinoma, colon cancer, lung cancer, lymphoma, leukemia, melanoma, basal cell cancer, cervical cancer, colorectal cancer, stomach cancer, bladder cancer, anal cancer, bone cancer, brain tumor, esophageal cancer, gall bladder cancer, gastric cancer, testicular cancer, Hodgkin's Lymphoma, intraocular melanoma, kidney cancer, oral cancer, melanoma, neuroblastoma, Non-Hodgkin's Lymphoma, ovarian cancer, retinoblastoma, skin cancer, bucal cancer, throat cancer, and thyroid cancer. Fibroblasts having proximity to any of the aforementioned cancer types or grown in a culture comprising such cancer cells are termed cancer-associated fibroblasts (CAFs). For example, breast cancer associated fibroblasts are those growing in a culture that also contains cancer cells, in particular, breast cancer cells.

As used herein, an individual “having” or “suffering from” a disease, disorder, or condition means that the person has been diagnosed with or displays one or more symptoms of the disease, disorder, or condition

By “nucleic acid molecule” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced stability in the presence of nucleases. In certain embodiments, the term “nucleic acid molecule” refers to genetic material that can be transferred via EV including, but not limited to, mRNA, tRNA, rRNA, siRNA, shRNA, miRNA, DNA (including fragments, plasmids, and the like). Such genetic materials can be transferred to EV via transfection, transformation, electroporation, and microinjection.

By “obtaining” as in “obtaining the inhibitory nucleic acid molecule” is meant synthesizing, purchasing, or otherwise acquiring the inhibitory nucleic acid molecule.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “positioned for expression” is meant that a polynucleotide is positioned adjacent to a DNA sequence that directs transcription and translation of the sequence (i.e., facilitates the production of a given polynucleotide molecule.).

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

“Recombinant,” “recombinantly produced,” or “recombinantly expressed” refers to a molecularly manipulated form of a protein encoded by a polynucleotide sequence and produced in suitable host cells (cells transfected, transformed, transduced to express and produce the protein) by methods routinely practiced in the art. The protein-encoding polynucleotide sequence is typically contained in an expression vector and system (e.g., plasmid/bacterial, viral, or insect expression vector system) that expresses and produces the recombinant protein inside the cell. Polynucleotides, vectors and related methods are described infra. Recombinant protein is protein encoded by a gene (e.g., TGF-β, isoforms of TGF-β, or modified forms of TGF-β or its isoforms) that has been cloned into an expression vector system that supports expression of the gene, production of mRNA and translation of mRNA into protein inside the host cell. Proteins recombinantly expressed in eukaryotic expression systems may contain post-translational modifications such as glycosylation or phosphorylation.

By “reference” is meant a standard or control condition.

By “reporter gene” is meant a gene encoding a polypeptide whose expression may be assayed; such polypeptides include, without limitation, glucuronidase (GUS), luciferase, chloramphenicol transacetylase (CAT), and beta-galactosidase.

By “selectively deliver” is meant that the majority of the EV with membrane-tethered TGF-β is delivered, or delivers an agent, e.g., polypeptide, polynucleotide, to a target cell or cell type relative to non-target cells present in the culture, tissue, or organ. In embodiments, greater than about 50%, 60%, 70%, 80%, 90%, 95% or even approaching 100% of the EV with membrane-tethered TGF-β are delivered to a desired cell type. In other embodiments, only about 10%, 15%, 20% 25%, 30%, 35%, or 40% of the EV with membrane-tethered TGF-β are delivered to non-target cells.

As used herein, the term “stromal cell” refers to non-vascular, non-inflammatory, non-epithelial connective tissue cells of any organ that surround a tumor. Stromal cells are also known as cancer-associated fibroblasts. Stromal cells support the function of the parenchymal cells of that organ. Fibroblasts and pericytes are among the most common types of stromal cells. The stromal cells can be derived from numerous body tissue types, including, but not limited to, breast tissue, thymic tissue, bone marrow tissue, bone tissue, dermal tissue, muscle tissue, respiratory tract tissue, gastrointestinal tract tissue, genitourinary tissue, central nervous system tissue, peripheral nervous system tissue, reproductive tract tissue. In an embodiment, stromal cells include mesenchymal stromal cells (MSC).

As used herein, the term “subject” refers to a human or any non-human animal (e.g., mouse, rat, rabbit, dog, cat, cattle, swine, sheep, horse, goat, camel, llama, or primate). A human includes pre- and post-natal forms. In many embodiments, a subject is a human being. A subject can be a human patient who presents to a medical provider for diagnosis or treatment of a disease. A subject can also be a human patient who presents to a medical provider as being at risk (short-term or long-term risk) for having a disease. The term “subject” is used herein interchangeably with “individual” or “patient.” A subject can be afflicted with or susceptible to a disease or disorder, but may or may not display symptoms of the disease or disorder.

The term “pharmaceutically-acceptable excipient” as used herein means one or more compatible solid or liquid filler, diluents or encapsulating substances that are suitable for administration into a human.

By “specifically binds” is meant a molecule (e.g., peptide, polynucleotide) that recognizes and binds a protein, such as TGF-β tethered to an EV membrane according to the invention, but which does not substantially recognize and bind other molecules in a sample, for example, a biological sample (e.g., a biofluid or tissue sample), which naturally includes the protein.

By “sample” is meant any body fluid or biofluid that is obtainable from a subject (donor), including, but not limited to, blood, plasma, serum, tears, saliva, sputum, urine, stool (feces), semen, cerebrospinal fluid, prostate fluid, lymphatic drainage, bile fluid, peritoneal fluid and pancreatic secretions. A sample may also embrace a cell, tissue, or organ sample, which may be suitably processed and/or reconstituted or resuspended in a suitable buffer, carrier, diluent, or vehicle for ease of manipulation and analysis.

By “substantially identical” is meant a protein or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and still more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “targets” is meant is specific for, delivered to, and/or alters the biological activity of a target, e.g., a polypeptide, nucleic acid molecule, cell, or tissue.

By “tethered” is meant attached or bound to, or expressed on, the membrane surface of an extracellular vesicle (EV). Accordingly, tethered TGF-β is bound to the membrane of an extracellular vesicle (EV) derived or originating from a cell, e.g., a MSC. TGF-β is typically bound to the membrane of EV derived from a cell via linkage to glycoproteins such as beta-glycans or via heparin molecules. The terms “membrane-tethered TGF-β EV,” “EV comprising membrane-tethered TGF-β,” “tethered TGF-β EV,” “EV having TGF-β tethered to the membrane,” “TGF-β tethered EV,” “EV expressing membrane-tethered TGF-β” and the like, are used interchangeably herein to refer to extracellular vesicles (EV) with TGF-β tethered (bound to) the membrane surface. In an embodiment, “MSC-derived, membrane tethered-TGF-β EV” refers to extracellular vesicles (EV) that are derived or originated from, or produced by, mesenchymal stromal cells (MSC) and that have TGF-β tethered (bound to) the membrane surface.

By “transformed cell” is meant a cell into which (or into an ancestor of which) has been introduced, by means of recombinant DNA techniques, a polynucleotide molecule encoding a protein of interest.

By “transforming growth factor beta” or “TGF-β” is meant a polypeptide member of the transforming growth factor beta superfamily of cytokines. It is a secreted protein that performs (or is involved in) many cellular functions, including the control of cell growth, cell proliferation, cell differentiation and apoptosis. The TGF-β superfamily includes four different TGF-β isoforms (TGF-β 1 to 4 (TGF-β1, TGF-β2, TGF-β3, TGF-β4)), as well as many other signaling proteins produced by different types of leukocytes. Activated TGF-β complexes with other factors to form a serine/threonine kinase complex that binds to TGF-β receptors, which are composed of both type 1 and type 2 receptor subunits. After TGF-β binds, the type 2 receptor kinase phosphorylates and activates the type 1 receptor kinase, which, in turn, activates a signaling cascade. This leads to the activation of different downstream substrates and regulatory proteins, inducing transcription of different target genes that function in differentiation, chemotaxis, proliferation and the activation of many immune cells (Massague, J., 2012, Nature Reviews. Mol. Cell Biol., 13(10):616-630; Nakao, A. et al., 1997, Nature, 389(6650:631-635).

TGF-β is secreted by many cell types, including macrophages, in a latent form in which it is complexed with two other polypeptides, latent TGF-β binding protein (LTBP) and latency-associated peptide (LAP). Briefly, TGF-β1, TGF-β2 and TGF-β3 are synthesized as precursor molecules containing a propeptide region in addition to the TGF-β homodimer. After it is synthesized, the TGF-β homodimer interacts with a Latency Associated Peptide (LAP), which is derived from the N-terminal region of the TGF-β gene product, and forms a complex called a Small Latent Complex (SLC). This complex remains in the cell until it is bound by another protein called Latent TGF-β-Binding Protein (LTBP), which is a high molecular weight, protease resistant binding protein. LTBPs are required for the proper folding and secretion of TGF-β. The SLC bound to the LTBP forms a larger complex called Large Latent Complex (LLC), which is secreted to the extracellular matrix (ECM). In most cases, before the LLC is secreted, the TGF-β precursor is cleaved from the propeptide but remains attached to it by noncovalent bonding. After its secretion, it remains in the extracellular matrix as an inactivated complex containing both the LTBP and the LAP, which are further processed in order to release active TGF-β. (J. P. Annes et al., 2003, J. Cell Sci., 116, pp. 217-224). TGF-β is attached to the LTBP is by disulfide bonding, which allows it to remain inactive by preventing it from binding to its receptor(s). Release of these complexes and activation by proteases is under tight regulation and provides a means to rapidly increase local concentrations of TGF-β. (Koli, K. et al., 2001, Microsc Res Tech, 52(4):354-362). Because different cellular mechanisms require distinct levels of TGF-β signaling, the inactive complex of the TGF-β cytokine allows for a proper mediation of TGF-β signaling. Serum proteinases such as plasmin catalyze the release of active TGF-β from the complex. This occurs, for example, on the surface of macrophages where the latent TGF-β complex is bound to CD36 via its ligand, thrombospondin-1 (TSP-1). Inflammatory stimuli that activate macrophages enhance the release of active TGF-β by promoting the activation of plasmin. Macrophages can also endocytose IgG-bound latent TGF-β complexes that are secreted by plasma cells and then release active TGF-β into the extracellular fluid. TGF-β plays key roles in regulating inflammatory processes, particularly in the gut; in stem cell differentiation; and in T-cell regulation and differentiation. (Massague, J., 2012, FEBS Letters, 586(14):1953-1958; Li, M. et al., 2008, Cell, 134(3):392-404). The TGF-β superfamily includes endogenous growth inhibiting proteins. An increase in TGF-β expression has been found to correlate with the malignancy of many cancers and a defect in the cellular growth inhibition response to TGF-β, which contributes to oncogenesis. The disregulation of TGF-β's immunosuppressive functions is also implicated in the pathogenesis of autoimmune diseases.

The TGF-β isoforms have highly similar peptide structures with about 70-80% sequence identity. All are encoded as large protein precursors with an N-terminal signal peptide of 20-30 amino acids; TGF-β1 contains 390 amino acids, while TGF-β2 and TGF-β3 each contain 412 amino acids. As noted supra, the isoforms have a pro-region called latency associated peptide (LAP; also called “Pro-TGF beta 1” or “LAP/TGF beta 1”) and a C-terminal region of about 112-114 amino acids that becomes the mature TGF-β molecule following its release from the pro-region by proteolytic cleavage. The mature TGF-β protein dimerizes to produce a 25 KDa active protein with many conserved structural motifs. TGF-β has nine cysteine residues that are conserved among its family members. Eight of the cysteines form disulfide bonds within the protein to create a cysteine knot structure characteristic of the TGF-β superfamily. The ninth cysteine forms a disulfide bond with the ninth cysteine of another TGF-β protein to produce a dimer (Daopin, S. et al., 1992, Science, 257(5068):369-373). Many other conserved residues in TGF-β are thought to form secondary structure through hydrophobic interactions.

By “vector” is meant a nucleic acid molecule, for example, a plasmid, cosmid, or bacteriophage, which is capable of replication in a host cell. In one embodiment, a vector is an expression vector that is a nucleic acid construct, generated recombinantly or synthetically, bearing a series of specified nucleic acid elements that enable transcription of a nucleic acid molecule in a host cell. Typically, expression is placed under the control of certain regulatory elements, including constitutive or inducible promoters, tissue-preferred regulatory elements, and enhancers.

The singular terms “a,” “an,” and “the” include the plural reference unless context clearly indicates otherwise. Similarly, the word “or” is intended to include “and” unless the context clearly indicates otherwise.

### BRIEF DESCRIPTION OF THE FIGURES

FIG. 1 presents a graph showing that inhibition of TGF-β reverses the antiproliferative effects of WJ-EV on CD4+ cells.

FIG. 2 presents a flow cytometry plot showing the results of flow cytometry analysis to assess the tethering of TGF-β to the surface of extracellular vesicles derived from mesenchymal stem cells.

FIG. 3 presents a flow cytometry plot showing the results of flow cytometry analysis to assess TGF-β tethered to the membrane surface of extracellular vesicles in plasma.

FIGS. 4A-4D present microscope images and graphs related to characteristics of Wharton's Jelly mesenchymal stromal cells (MSC) from which EV may be derived as described and exemplified herein. Wharton's Jelly MSC in culture display typical stromal cell phenotype. FIG. 4A shows WJ-MSC (passage 5) expanded in complete alpha-MEM (see, e.g., Example 5, Methods); colony formation and mesenchymal stromal cell morphology; FIG. 4B shows expression of genes encoding surface markers that define the immunophenotype of MSC, including CD44, CD73, CD90, and CD105 (n=3 technical replicates/sample, n=3 cell line samples); FIG. 4C shows expression of surface proteins typically expressed on MSC including CD44 and CD90. Low level expression of CD34 was observed (isotype (leftmost tracing), antigen (rightmost tracing), 2 representative WJ-MSC cell lines, 30,000 events per sample. FIG. 4D presents microscope images demonstrating that WJ-MSC exhibited the capacity for differentiation to osteocyte, chondrocyte and adipocyte cell types.

FIGS. 5A-5E present nanoparticle tracking analysis (NTA), transmission electron microscopy, density, and Western blot results showing that extracellular vesicles derived from WJ-MSC through stepwise ultracentrifugation demonstrate typical morphology consistent with exosomes. FIG. 5A: WJ-MSC EV were isolated by differential ultracentrifugation from WJ-MSC conditioned medium. The mean (+SEM) of particle size distribution of samples of isolated EV was measured (mode 125 nm, mean 199 nm, n=5 cell lines pooled: WJ-MSC EV 12, 58, and 85). FIG. 5B: Ultrastructural appearance by transmission electron microscopy of EV isolated from WJ-MSC conditioned; left panel=3870× magnification, scale bar 200 nm; right panel=4135× magnification, scale bar 100 nm. FIG. 5C: WJ-MSC EV were applied to an iodixanol density gradient, and particle density from fractions 1-8 (n=3: as above cell lines 12, 58, and 85), histograms and total protein by BCA for each fraction are shown. FIG. 5D: Western blot results for EV specific marker TSG101 (46 kDa MW expected) maximal in fractions 2 and 3 (n=3 cell lines pooled as above) following iodixanol density gradient separation. FIG. 5E: WJ-MSC EV isolated by stepwise ultracentrifugation expressed TSG101 (expected MW 46 kDa), Alix (expected MW 96 kDa), but not the cellular endoplasmic reticulum origin protein calnexin (expected MW 90 kDa); 12.5 μg/lane, except HeLa lysate 10.0 μg loading, with primary antibody employed at a concentration of 1:1000.

FIGS. 6A-6E present graphs showing that WJ-MSC EV suppress CD4+ (CD4^{pos}) T cell division. FIG. 6A: WJ-MSC EV isolated from WJ-MSC culture supernatant demonstrated a dose-dependent suppression of CD4^{pos} cell division. CD4^{pos} T cell divisions were suppressed in the presence of WJ-MSC co-cultured with PBMC across a transwell (1 MSC:10 PBMC). PBMC exposed to WJ-MSC EV (10⁴ EV:1 PBMC) reduced CD4^{pos} T cell division, which was not statistically different from the effect of parent WJ-MSC cell lines. CD4^{pos} T cell division in the presence of EV supernatant or EV isolated by stepwise ultracentrifugation from canine cardiac fibroblast conditioned medium (10⁴ EV:1 PBMC) was not significantly different from controls (no MSC or EV). FIG. 6B: Pretreatment of WJ-MSC with GW4869 (6 μM, 48 hr) significantly reduced their ability to suppress PBMC division. FIG. 6C: CD4^{pos} T cell suppression was due to WJ-MSC EV, and not to soluble factors co-sedimented with EV, as shown by the failure of filtrate (10 or 50 kDa MWCO) derived from EV sediment to suppress CD4^{pos} T cell division. FIG. 6D: Pre-treatment of WJ-MSC EV with Triton-X (0.1%) abolished CD4^{pos} T cell suppression; RNase or Proteinase K pretreatment partly reversed the suppressive effect of WJ-MSC EV, but to a significantly lesser extent than Triton-X. FIG. 6E: Biological activity across donor cell lines for WJ-MSC (Top, 1 MSC:10 PBMC) or WJ-MSC EV (Bottom, 10⁴ EV:1 PBMC).

FIGS. 7A-7C demonstrate that increasing concentrations of GW4869 decreased release of WJ-MSC-EV (A, n=3 WJ-MSC Lines) and that Interferon-γ (IFN) pretreatment of WJ-MSC did not significantly increase suppressive activity of WJ-MSC or WJ-MSC EV. FIG. 7A: Average number of particles released in 48 hours at different concentrations of GW4869. FIG. 7B: NTA histograms of size distribution of particles between various GW4869-concentration treated WJ-MSC EV (B, red, green, and black histograms) compared to untreated WJ-MSC EV, showing a reduction in EV numbers across size range (50-200 nm). FIG. 7C: WJ-MSC were pretreated with 500 ng IFN-γ for 48 hours prior to EV collection or co-culture with PBMC across a Transwell.

FIGS. 8A-8C present results showing that TGF-β and adenosine are two mechanisms by which WJ-MSC EV suppress CD4^{pos} T cell division. FIG. 8A shows that neutralizing antibody against mature TGF-β, inhibition of TGF-βRI, or adenosine A2A receptor antagonism significantly reduced WJ-MSC EV mediated CD4^{pos} T cell suppression. FIG. 8B shows a Western blot depicting the presence of TGF-βRI in PBMC (lanes 1-4) and CD4^{pos} T cell lysates (lanes 6 and 7); also shown are HEK293 cell lysate (positive control, lane 8) and bovine serum albumin (negative control, lane 9); 2.6 μg protein loaded per lane, FIG. 8C shows that exogenous TGF-β1 or TGF-β3 (10 or 50 ng/ml, but not 5 ng/ml) suppressed CD4^{pos} T cell division in a manner comparable to the effects of WJ-MSC or WJ-MSC EV.

FIGS. 9A-9D present ELISA analysis, Western blot and cell division assay results. FIGS. 9A and 9B present results of ELISA analysis showing TGF-β1 concentrations (ng/ml or pg/ml) of WJ-MSC EV prior to and after acid activation demonstrating that concentrations per EV and concentrations per 5×10 EV as applied to each PBMC responder well. FIG. 9C shows a Western blot of TGF-β detected in samples of WJ-MSC EV. WJ-MSC lysate (CL) was compared to WJ-MSC EV in non-reducing (−) and reducing (DTT) (+) conditions. Bands at ˜150 kDa represent TGF-β large latent complexes, while bands at ˜50 kDa or ˜75 kDa represent TGF-β pro-form including LAP; recombinant human mature TGFβ1 is visible as a homodimer (˜25 kDa). The Western blot analysis demonstrated that EV-associated TGF-β was detected only at the molecular weight of the large latent complexes and pro-form, with the mature TGF-β homodimer (˜25 kDa) detected only after DTT reduction of the samples. FIG. 9D shows that inhibition of TGFβRI inhibitor and heparinase-treatment of EV both significantly reduced EV-mediated suppression of CD4^{pos} T cell division, an effect which was abolished by pretreatment of EV with heat-inactivated heparinase (HI heparinase), consistent with an association between membranous TGF-β and co-receptor beta-glycan and activation requiring intact heparin side chains.

FIGS. 10A-10C present results of treatment of MSC EV with hyaluronidase and analysis of the treated particles by SEC-HPLC, Nanotracking particle analysis (NTA) or Western blot (WT).

### DETAILED DESCRIPTION OF THE INVENTION

The invention provides methods for disease assessment, evaluation, diagnosis and monitoring involving extracellular vesicles (EV) derived from a cell source, such as, without limitation, a cancer cell, cancer associated cell (e.g., fibroblast-like cell, cancer-associated fibroblast), a dendritic cell, stromal cell, or stromal stem cell, having tethered to the membrane (i.e., membrane bound) an immunomodulatory molecule (e.g., polypeptide, polynucleotide, small molecule). In a particular embodiment, the cell source is a mesenchymal stromal cell or “MSC” (also called a “meschenchymal stem cell”). In another particular embodiment, the membrane-tethered molecule is TGF-β or an isoform thereof, e.g., TGF-β1-4. In another particular embodiment, the cell source is an MSC and the extracellular vesicles (EV) are MSC-derived and comprise TGF-β or an isoform thereof, e.g., TGF-β1, tethered to the membrane surface.

The present invention is based, at least in part, on the finding that TGF-β is tethered (i.e. membrane bound) to the membrane of extracellular vesicles (EV) derived from a number of different cell types, including, by way of example, cancer cells, cancer associated cells (e.g., fibroblast-like cells, cancer-associated fibroblasts), dendritic cells, stromal cells, e.g., mesenchymal stromal cells (MSC), or stromal stem cells, and the recognition that MSC-derived EV expressing membrane-tethered TGF-β play a critical role in immunomodulation of diseases and conditions, and immunomodulatory activity, and therefore, also play a critical role in the potency of cells such as MSC. For example, EV having membrane-tethered TGF-β, such as MSC-derived TGF-β having membrane-tethered TGF-β, can have profound immunosuppression and anti-inflammatory actions, particularly in subjects having a disease or condition, such as an inflammatory disease, an autoimmune disease, transplant rejection, or cancer. Such TGF-β-tethered MSC EV can exert an immunosuppression effect that is counterproductive in an individual's defense against disease or the treatment thereof, for example, cancer or cancer treatment, as well as in the effectiveness of EV-based vaccines.

Accordingly, provided by the invention are extracellular vesicles (EV) derived from a cell type (e.g., cancer associated cells, fibroblast-like cells, stromal cells, stromal stem cells, dendritic cells, cancer cells, MSCs, or other cells that originate from a site of immune privilege), that express TGF-β or an isoform thereof (e.g., TGF-β1, TGF-β2, TGF-β3, or TGF-β4) tethered to the membrane of the EV, and methods in which such TGF-β-tethered EV are used in diagnosis, determining immune status, monitoring therapy or treatment of disease, or assessing, evaluating or diagnosing a disease or condition in a subject or in a patient population. In other embodiments, another immunomodulatory agent, (e.g., polypeptide, polynucleotide, small molecule), in addition to or instead of TGF-β is tethered to the membrane of the EV. Methods are also provided to quantify TGF-β on EV as a benchmark of disease activity (e.g., a standard against which disease activity is measured, evaluated, or compared) in a subject or in a patient population, e.g., a subject or patient population undergoing treatment or therapy for a disease, e.g., an inflammatory disease, an autoimmune disease, transplant rejection, heart disease, or cancer. In addition, methods of altering or controlling the amount, level, or density of TGF-β tethered to the EV membrane are encompassed by the invention, as described herein. Also encompassed by the invention are the resulting membrane-tethered TGF-β EV, which can be used as therapeutic products in patients with a number of diseases and conditions.

In accordance with the invention, the measurement of tethered TGF-β on EV membrane provides an advantageous approach to assessing, monitoring, or benchmarking the immune status of human or animal (veterinary) patients. As used herein, “benchmarking” refers to methods and approaches for comparing to best practices, novel technologies, or other gold standard technologies or assays for assessing immune status. The accurate quantification of membrane-tethered TGF-β (or other immunomodulatory proteins) on the EV in a biofluid obtained from a subject (patient) provides an improved index or determination of disease activity, aggressiveness, prognosis, and/or response to therapy, or other aspects of the natural history or the course of a disease.

In addition, because extracellular vesicles (EV) having membrane-tethered TGF-β are biologically active, they provide an actionable target which may be useful for increasing or restricting activity related to a disease. For example, in instances in which the fraction of EV with membrane-tethered TGF-β, or the level of membrane-tethered TGF-β expression per EV, in a subject is lower than expected based on quantification as described herein and comparison with a control or reference (e.g., reference ranges for a particular cohort), reduced immunosuppression (e.g. in autoimmune disease, chronic inflammation, allergy) may be indicated, thereby leading to the supplementation of EV having membrane-tethered with TGF-β in the subject. In an embodiment, the EV having membrane-tethered TGF-β can be selected or isolated from total EV derived from MSC as described herein. Conversely, a finding of an excessive level of MSC-derived EV having membrane-tethered TGF-β in a subject (e.g., a subject having cancer) as quantified by the methods described herein can prompt interventions that neutralize the tethered TGF-β in the subject, thus specifically restoring immune activity. Nonlimiting examples of auto-immune diseases and disorders associated with a low concentration, amount, or level of membrane-tethered TGF-β on EV include asthma, atopic dermatitis, inflammatory bowel diseases, psoriasis, multiple sclerosis, or lupus. Nonlimiting examples of immunosuppression diseases and conditions that are associated with a high concentration, amount, or level of membrane-tethered TGF-β on EV include cancer or drug induced (chemotherapeutic) or radiation induced immunosuppression.

Pertinent to therapeutics and the methods described herein, the accurate quantification of immunomodulatory protein tethered to the membrane of EV produced by immunotherapeutic MSC or other cell types, e.g., membrane-tethered TGF-β EV derived from MSC, allows for improved selection criteria for EV potency to improve patient therapy or treatment. In an embodiment, membrane-tethered TGF-β provides a target for direct isolation of a specific subset of EV with high levels of membrane-tethered TGF-β (or other immunomodulatory molecules). Such EV comprising membrane-tethered TGF-β can be used as immunosuppressive agents, for example, if the isolated membrane-tethered TGF-β EV are administered to a subject or to a cell culture. In another embodiment, membrane-tethered TGF-β provides a target for direct isolation of a specific subset of EV with high levels of membrane-tethered TGF-β (or other immunomodulatory molecules) from a biological sample or cell culture, so as to deplete EV with membrane-tethered TGF-β (or other immunomodulatory molecules) from the sample, Accordingly, EV obtained from a sample depleted of EV with a high level of membrane-tethered TGF-β can be used as a therapeutic in a subject or cell culture, for example, to reduce the immunosuppressive effect of EV having high level of membrane-tethered TGF-β. In another embodiment, EV having membrane-tethered TGF-β provide a target for direct isolation of a specific subset of EV with low levels of membrane-tethered TGF-β (or other immunomodulatory molecules).

Extracellular Vesicles with Membrane-Tethered (Membrane Bound) TGF-β

Extracellular vesicles (EV) are nanoscale membrane-bound structures originating from early endosomes, which are released by all cells as part of the paracrine system of intercellular communication (Yanez-Mo, M. et al., 2015, J. Extracell. Vesicles, Vol. 4:27066). The membranous and internal compartment of EV contains bioactive molecules including RNA, DNA, protein, and lipids. EV from dendritic cells, T regulatory cells, tumor cells, tumor stromal cells, and mesenchymal stem cells impart immunosuppressive effects on recipient cells (Zhang, B. et al., 2014, Front. Immunol., Vol 5:518). The immunotherapeutic potential possessed by mesenchymal stem cells (MSC) and extracellular vesicles (EV) derived from MSC may be associated with their expression of various proteins, including TGF-β, PD-LI, Galectin 1, PGE2, CD73 and CD39, IDO, and IL-10 (Bruno, S. et al., 2015, Immunol. Lett., Vol. 168(2):154-158). As embraced by the invention and as will be appreciated by the skilled practitioner, TGF-β represents a class of immunomodulatory molecules that can be tethered to the membrane of extracellular vesicles (EV), alone or in combination with other molecules, for example and without limitation, PD-L1, FasL, and Galectin-1.

While soluble TGF-β protein present in plasma or serum may indicate chronic inflammatory or autoimmune diseases (e.g., psoriasis) or fibrotic conditions (e.g. interstitial pneumonia), it is believed that the use of TGF-β tethered to the EV membrane was not known or considered to be an effective, quantifiable molecule or biomarker for any disease or condition until the present invention. Tethered TGF-β comprises only a fraction of the total TGF-β in biofluids or cell culture supernatants; yet TGF-β tethered to the EV membrane is more bioactive than soluble TGF-β. By way of example, membranous TGF-β on EV from tumor cells induced a cancer-like phenotype (e.g. pro-angiogenic, tumor promoting) in myofibroblasts, while soluble TGF-β did not (Webber, J. P. et al., 2015, Oncogene, Vol. 34(3):290-302). Thus, the invention provides advantageous methods involving quantification of TGF-β tethered to the membrane of extracellular vesicles (EV) as a more bioactive form of TGF-β for the assessment and evaluation of disease status or for the provision of disease therapy or treatment in a subject who is afflicted with a disease. In embodiments, the various isoforms of TGF-β tethered to the EV membrane, namely, TGF-β1, TGF-β2, TGF-β3 or TGF-β4, can be quantified and assessed or evaluated as a disease biomarker in accordance with the described methods, which provide an improvement on current processes that consider only total serum or plasma levels of soluble TGF-β. In a particular embodiment, EV having membrane-tethered TGF-β1 serves as a biomarker that may be quantified and assessed as correlating with a number of diseases, or the status thereof, including cancer and non-cancer diseases and conditions, for example, cancers of the types described herein, as well as autoimmune diseases (e.g., psoriasis, arthritis, multiple sclerosis, system sclerosis, amyotrophic lateral sclerosis (ALS)), inflammatory diseases, transplantation rejection, myocardial infarction; and coronary disease. In another embodiment, the quantification of TGF-β tethered to EV from MSC can be used to determine the potency of MSC cell lines, thereby enabling the selection or isolation of MSC cell lines that are more immunosuppressive by virtue of their production of membrane-tethered TGF-β EV that are determined to have a higher concentration of tethered TGF-β. In another embodiment, tethered TGF-β EV produced by such MSC can be isolated and administered as a therapeutic having immunosuppressive function in disease treatment for a subject in need.

### Extracellular Vesicles (EV) Comprising Membrane-Tethered TGF-β—Methods of Detection, Isolation and Use

TGF-β tethered to the membrane of a variety of cell types, and particularly, TGF-β tethered to the membrane of extracellular vesicles (EV) derived from these cell types, e.g., cancer-associated fibroblasts, stromal cells, dendritic cells, mesenchymal stromal cells, and cells obtained from sites of immune privilege as described herein by way of nonlimiting example, provides a measurable and specific, disease-associated target for use in the methods described and provided herein. In a certain embodiment, isolated EV derived from mesenchymal stromal cells (MSC) and comprising membrane tethered TGF-β are particularly suitable for the uses as described herein. In another embodiment, the EV are treated with hyaluronidase to remove hyaluronic acid/proteoglycan complexes on their surfaces during the isolation process, for example, centrifuged or sedimented EV may be treated with hyaluronidase at the time of resuspending the centrifuged pellet containing EV (See, e.g., Example 5, infra).

In embodiments of the methods, TGF-β (including isoforms TGF-β1, TGF-β2, TGF-β3 and/or TGF-β4) on the surface of extracellular vesicles (EV), e.g., tethered to the EV surface membrane via beta-glycan, (also referred to as TGF-βR3), can be quantified (measured) in a biological sample from a subject or in cell culture using several different methods. In an embodiment, single vesicle nanoparticle tracking analysis can be used in which TGF-β is immunolabeled by QDOT® conjugated antibody or indirect labeling (e.g. biotin-antibody, streptavidin-QDOT) is used to quantify TGF-β tethered to the membrane of EV. According to this method, membrane-tethered TGF-β EV are labeled using a biotinylated primary antibody (e.g., anti-human TGF-β/LAP, clone: CH6-17E5.1 (Miltenyi Biotec Inc., San Diego, Calif.), incubating at 4° C. for from 10-60 minutes, or for 20-45 minutes, or for at least 30 minutes, or for 30 minutes, washing by ultracentrifugation (100,000×G, 120 min, 70Ti rotor) to remove unbound antibody, filtering with 0.22 um filter to remove aggregates if necessary, and labeled secondarily with streptavidin conjugated quantum dots (655 nm emission, QDOT, ThermoFisher, Waltham, Mass.). Unbound streptavidin-QDOTS (˜20 nm diameter) are separated from labeled EV using size exclusion chromatography (HPLC, e.g. Agilent 1100, column: AdvanceBio SEC-5, 300 \dot{A}, 2.7 um, 7.8×300 mm, mobile phase pH 7.4 PBS, flow rate 0.5 ml-1.0 ml/min), based on UV absorbance at 220 nm or 280 nm. QDOT separation is confirmed using fluorescence detection (488 nm excitation/655 nm emission) coupled to the HPLC instrument, The percentage of membrane-tethered TGF-β EV that are QDOT labeled (TGF-β^{pos}) is quantified. The intensity of TGF-β expression on EV of different sizes can also be evaluated. Mathematical corrections for subdiffusion may be necessary to obtain accurate particle size distribution; however, this does not interfere with the measurement of single vesicle expression of TGF-β. Total EV in a sample used as the denominator, can be identified (and distinguished from protein complexes including lipoproteins) based on staining with EV-specific or EV-enriched surface markers such as CD9, CD63, CD81, LAMP-2, heat shock proteins, Alix, synectin, or flotillin, or by the use of fluorogenic dyes, or molecular beacons, or by staining the nucleic acid cargo of the EV.

In another embodiment, vesiculometry employing fluorescence detection of immunolabeled EV or those absorbed to beads can be used to quantify membrane-tethered TGF-β EV. In another embodiment, TGF-β and isoforms thereof tethered to the membrane of EV, such as EV derived from MSC, can be quantified in a subject's biological sample, e.g., blood or plasma, or in a cell culture by a method, e.g., an interferometry method, that does not require the isolation of EV from the sample or culture.

Interferometry is an example of a system used by the skilled practitioner that utilizes fluidics to analyze very small volumes of plasma or serum. In general, antibodies that bind to TGF-β (e.g. anti-TGF-β antibodies) are used to capture and immobilize EV with membrane-tethered TGF-β, e.g., in a well of an assay plate; this binding ‘interferes’ with the transmission of light at that location in the well (Daaboul, G. G. et al., 2016, Scientific Reports, 6, Article number:37246). In an embodiment, a direct “immunocapture” method can be utilized for the enumeration of EV with membrane-tethered TGF-β in which specific antibodies, e.g., anti-TGF-β antibodies (“anti-TGF-β capture antibodies”) are attached to a substrate or solid phase, e.g., a chip, membrane, or film. A biological sample or culture supernatant (culture medium) is contacted with the substrate, and TGF-β tethered to the EV membrane binds directly to the capture antibodies. For example, the sample or supernatant can be contacted with the substrate and capture antibodies for 10-60 minutes, for 20-45 minutes, for at least 30 minutes, or for 30 minutes, at room temperature or at 4° C. Thereafter, light interference measurement is used to determine the amount of TGF-β (membrane-tethered TGF-β) that is bound to the anti-TGF-β antibodies on the film (e.g., NANO-VIEW® nano- and micro-positioners, MCL, Madison, Wis.). The use of anti-TGF-β antibodies to capture EV comprising membrane-tethered TGF-β allows for direct assay of EV with TGF-β tethered to the membrane surface, including all TGF-β isoforms. The technique of interferometry (e.g., attachment to a chip or membrane) can be used to quantify the frequency with which an ‘epitope,’ such as membrane-tethered TGF-β, can be found on EV. The membrane-tethered form of TGF-β can be distinguished from soluble non-membrane-tethered TGF-β, because the soluble form does not ‘interfere’ with light sufficiently to be counted as an EV having membrane-tethered TGF-β, i.e., it is undetected by the system.

Accordingly, interferometry, vesiculometry (flow cytometry technology adapted for nanoparticle assessment), nanoparticle tracking analysis fluorescence or any method that assesses, determines, or benchmarks the quantity, phenotype and size distribution of EV with membrane-tethered TGF-β may be used to quantify TGF-β tethered to the membrane of EV. The measurement data are quantified relative to total EV, total protein, or total EV proteins (e.g. CD9, CD63, CD81, TSG101, flotillin, synectin, LAMP-2, or Alix), nucleic acids, lipids, or other constituents that represent the total EV population in a sample. In addition, the data may be used to stratify patient status by stage, aggressiveness, prognosis, resistance to therapy, or any aspect of disease status.

In an embodiment, membrane-tethered TGF-β EV are isolated or enriched from a subject's biological sample or from cell culture medium or supernatant. By way of example, biofluids samples such as blood, urine, cerebrospinal fluid, or saliva obtained from subjects (patients), or cell culture supernatants, are cleared of cells, platelets, apoptotic bodies, cell debris, protein aggregates, and other particulates that are not extracellular vesicles. This can be achieved by differential centrifugation (e.g., 1300×g for 10 minutes to remove cells and platelets, 2000×g for 10 minutes to remove apoptotic bodies, and 10,000×g for 30 minutes to remove microvesicles), or by sequential filtration after clarification of cells and apoptotic bodies using a 200 nm filter.

The measured levels of EV with membrane-tethered TGF-β isolated from a biological sample should fall within a reference range, above or below which is interpretable as ‘active disease’ or abnormal levels. The reference range can be determined from a subject having no disease (healthy or normal subject), from a subject having non-active disease, or from a sample obtained from the same subject at a first time point, or at an earlier, or pre-active disease, time point. In addition, a suitable reference or control can be the amount of membrane-tethered-TGF-β EV relative to total protein, EV specific proteins, or total EV numbers in the sample undergoing analysis. By way of example, a greater than or equal to 1.5 fold change, or an at least 1.5-fold change, in the level of membrane-tethered TGF-β EV in a sample undergoing testing or quantification and the reference (or control) sample, is indicative of a biologically relevant or significant difference in the quantification analysis. In embodiments, a change in the level of membrane-tethered TGF-β EV in a sample relative to a reference or control level of at least or equal to 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 10.5-fold, 11-fold, 12-fold, 15-fold, 20-fold, 25-fold, or greater, including values therebetween, is indicative of a biologically relevant or significant difference in the quantification analysis. Active disease is defined as disease with an abnormal, i.e., high, elevated, or low, level or amount of membrane-tethered-TGF-β EV or progression of this trend in a single patient. In an embodiment, the level or amount of membrane-tethered-TGF-β EV is increased or elevated relative to a control level or amount. Inactive disease implies normal membrane-tethered-TGF-β EV and normal immune function associated with this molecule. In an embodiment, an increased amount of membrane-tethered-TGF-β EV derived from MSC indicates superior immunosuppression potential of such membrane-tethered TGF-β EV as a therapeutic. Decreased amounts of membrane-tethered-TGF-β EV or on parent MSC indicates that the membrane-tethered TGF-β EV have low potential or low potency potential as a therapeutic. Hence, membrane-tethered-TGF-β EV, such as MSC-derived membrane-tethered TGF-β EV, can be used to select superior cells, e.g., MSC, for producing membrane-tethered TGF-β EV of high potency as a therapeutic. Alternatively, EV with decreased amounts of membrane-tethered-TGF-β, e.g., derived from MSC, or EV without any membrane-tethered-TGF-β, may be applicable for use, e.g., as a therapeutic, in immunosuppressive states to avoid compounding the native immunosuppressive state of the patient.

In an embodiment, membrane-tethered TGF-β EV are isolated or enriched from cultured cells, e.g., MSC. MSC isolated from biological samples are typically maintained in culture for 1 to 20 days, or for 48 hours, for collection of EV having membrane-tethered TGF-β. In general, MSC are maintained under standard culture conditions for cell growth, and are washed and transferred to serum free defined chemical medium, e.g. DMEM, L-glutamine (1 mM), and 5 ng/ml FGF2 and 5 ng/ml PDGF-AB glycoprotein (platelet derived growth factor-AB). Buffer (HEPES) is added to stabilize pH, e.g., pH 5-8.5, or pH 5.5-7.5, or pH 6-7.5, or pH 6-7. Other growth factors (e.g., EGF, 5 ng/ml) can be used to enhance the production and release of EV in certain cell lines. Immortalized MSC may be maintained in culture for 1-20 days and longer, e.g., for weeks or months with appropriate cell culture techniques. For cell culture supernatants, initial steps of concentration of EV are necessary, for example filtration (e.g., 200 nm pore size) to remove cells and cell debris, followed by tangential flow filtration (e.g. 50,000-300,000 kDa molecular weight cutoff (MWCO)). Extracellular vesicles are isolated from the clarified sample (e.g. plasma, serum, cell culture supernatant), for example, either by affinity column chromatography, tangential flow filtration (e.g., >50 kDa MWCO filter), precipitation (e.g., using PEG or ExoQuick, a polymer that gently precipitates extracellular vesicles, System Biosciences (SBI), Palo Alto, Calif.), differential ultracentrifugation (e.g., 100,000×g for 70 min using 70Ti rotor to sediment EV), density gradient centrifugation, size exclusion chromatography (e.g. 30-45 nm pore size), or other methods practiced in the art for the isolation and concentration of EV. In an embodiment, the EV, e.g., centrifuged or sedimented preparations of EV, are treated with hyaluronidase to remove hyaluronic acid/proteoglycan complexes on their surfaces.

In an embodiment, the biological activity of isolated EV comprising membrane-tethered TGF-β can be assayed. Assay of biological activity of TGF-β tethered to the EV membrane is achieved by any number of immunoassays (assays using immune cells), including assessment of the degree of suppression of (i) mitogen-induced (e.g., Concanavalin A, 5 ng/ml for 72 hr) T cell proliferation; (ii) CD3/CD28-induced T cell proliferation; (iii) T cell production of IFNγ or IL-17; (iv) CD69 expression by activated T cells; (v) differentiation or expansion of a T regulator cell subset; (vi) natural killer (NK) cell differentiation or activation; or (vii) maturation of dendritic cells (e.g., CD1a, MHCII, CD80, CD86 expression), using methods practiced by one skilled in the art. Tethered-TGF-β EV isolated from cell culture supernatant or patient samples (e.g., blood) can be assayed biologically to evaluate immunosuppressive function.

### Therapeutic Applications Involving TGF-β Tethered EV and Modified Forms Thereof

Quantification of the levels or amount of endogenous EV having membrane-tethered TGF-β relative to the total EV present in a biological sample obtained from a subject having a disease can be used by a physician or medical practitioner to decide on a specific therapy or treatment for the subject, in particular, a subject who is suffering from immunosuppression, e.g., during or as a result of treatment or therapy for a disease or condition. In an embodiment, “patient status” is defined as the level or amount of endogenous EV having membrane-tethered TGF-β relative to the total EV present in the patient's biological sample, and can be assessed, evaluated, identified and monitored via the methods involving membrane-tethered TGF-β EV as described herein. In an embodiment, a specific therapy directed by patient status includes a specific total dose (e.g., total number, concentration, route of administration) of EV expressing membrane-tethered TGF-β provided to the patient to achieve a desired outcome in the treatment of the patient's disease.

Examples of autoimmunity diseases, conditions, or disorders that are associated with low measured levels of TGF-β tethered to the EV membrane include asthma, atopic dermatitis, inflammatory bowel diseases, psoriasis, multiple sclerosis, rheumatoid arthritis, type 1 diabetes, graft versus host disease, sarcoidosis, Sjogren's Disease, or lupus. Low or decreased levels of membrane-tethered TGF-β EV may also be induced by drugs, radiation, trauma, or stress. In an embodiment, subjects (patients) who have or are determined to have low or decreased levels or amounts of membrane-tethered TGF-β EV are administered EV comprising membrane-tethered TGF-β to provide an immunosuppressive effect in the subject, for example, by direct intravenous, intra-spinal, intra-coronary, intra-lesional, topical, or other appropriate route of administration. The levels of exogenously supplied EV with membrane-tethered TGF-β are expected to rise transiently (e.g., for <24 hours), followed by a longer increase of EV with membrane-tethered TGF-β in circulating and tissue by recruitment of T regulatory cells and other cells that synthesize EV with membrane-tethered TGF-β.

While a primary cause of immunosuppression in patients with low levels of EV with membrane-tethered TGF-β (e.g., low levels in circulation or other biofluids) is cancer, there are also many examples of immunosuppression that is induced by other causes, such as viral infection, drugs, stress, trauma, degeneration, other infections. Without wishing to be bound by theory, in patients who are afflicted with diseases or conditions of these types, membrane-tethered TGF-β EV are expected to be disadvantageous; hence, interventions to reduce membrane-tethered TGF-β EV are preferred. Methods for reducing EV with membrane-tethered TGF-β or activity include, without limitation, serum neutralizing antibodies or molecularly-engineered or recombinantly produced antibodies, oligonucleotides (RNAi, anti-sense) to knock down membranous TGF-β, gene therapy, or pharmacological inhibitors of TGF-β, e.g., Smad inhibitors (e.g., SIS3, which inhibits Smad3), pirfenidone, or other commercially available TGF-β inhibitors. By way of example, Smad refers to a family of eight proteins that participate in tumor suppression in conjunction with TGF-β. Smad 1,2,3,5 and 8 are receptor-activated; Smad 4 is a co-mediator; and Smad 6 and 7 are inhibitory. The term ‘Smad’ is derived from the homology of these proteins to the Sma protein of Caenorhabditis elegans and the MAD proteins of Drosophila. Alternatively, the cell sources for membrane-tethered TGF-β EV can be reduced or eliminated by a method such as, for example, aphaeresis, targeted cytotoxicity, or chemotherapeutic agent treatment as known by the skilled practitioner. Reduction of EV with membrane-tethered TGF-β may be combined with approaches that reduce tumor or cancer cell burden. The recurrence of the primary source of EV with membrane-tethered TGF-β is reflected in rising levels of EV with membrane-tethered TGF-β in biofluid samples of subjects, e.g., patients who are being treated for cancer or who have been treated for cancer. Thus, EV with membrane-tethered TGF-β is a useful biomarker of residual cancer burden, recurrence, failure of cancer therapy, or resistance to therapy. Rising levels or amounts of membrane-tethered TGF-β EV is an indicator or biomarker of prognosis in cancer, with rising levels indicating or correlating with poorer prognosis or increased risk for or presence of malignancy or metastases.

In an embodiment, extracellular vesicles (EV) having membrane-tethered TGF-β are isolated, e.g., from primary or immortalized MSC in cell culture, and TGF-β tethered to the EV membrane may be substituted with one more different immunomodulatory molecules using routine molecular biological techniques. In an embodiment, one or more different immunomodulatory molecules may be tethered to the EV membrane in addition to tethered TGF-β. In another embodiment, an extracellular vesicle (EV) which has one of several immunomodulatory molecules tethered to the membrane can be produced by common molecular biological methods. Illustrative, yet nonlimiting examples of such immunomodulatory molecules that can be tethered with (or instead of) TGF-β on the membrane of EV include PD-1, PD-LI, B7-H4, B7-H5, CTLA-4, 4-1-BB, CD4, CD8, CD14, CD25, CD27, CD40, CD68, CD163, GITR, LAG-3, OX40, TIM3, TIM4, CEA, TLR, TLR2, etc., or cytokines, e.g., IL-4, IL-6, IL-7, IL-10, IL-12, IL-15, IL-17, IFN-γ, Flt3, BLys, chemokines, e.g., CCL21, or Galectin-1. By way of example, such additional molecules may enhance the activity of tethered TGF-β on MSC-derived EV having membrane tethered TGF-β when such EV are used as a therapeutic. Moreover, should such immunomodulatory molecules be tethered to the membrane of EV, in addition to membrane tethered TGF-β, they may be used as further markers in the detection or diagnostic the methods described herein.

In another embodiment, EV comprising membrane-tethered TGF-β (e.g., mesenchymal stromal cell (MSC)-derived or dendritic cell-derived EV) are isolated and TGF-β removed from the EV membrane using proteinases, glycanases, or heparinases. By way of example, dendritic-cell-derived EV lacking TGF-β can be used as antigen-presenting agents administered to a subject in need, e.g., as tumor vaccines, thereby alleviating or substantially alleviating immunosuppression associated with membrane-tethered TGF-β. In an embodiment, such dendritic cells, and, in turn, the EV derived therefrom, can be recombinantly modified to express certain tumor associated antigens to enhance immune cell response against tumors. In another embodiment, MSC-derived EV which lack membrane-tethered TGF-β, for example, the EV lacking membrane-tethered TGF-β can be negatively selected by immune affinity techniques. By way of example, this can be accomplished by depletion of membrane-tethered TGF-β EV from a mixture of EV, in which the mixture contains EV that have membrane-tethered TGF-β as well as EV that lack membrane-tethered TGF-β, using magnetic sorting (e.g., EV are incubated with anti-TGF-β antibodies conjugated to biotin, which is then incubated with secondary streptavidin bound to magnetic beads, which is used to remove EV with TGF-β tethered on the membrane surface magnetically (e.g. using an AUTO-MACS® cell separation device, Miltenyi Biotec Inc., San Diego, Calif.), while leaving EV that do not contain measurable tethered TGF-β in the depleted fraction for utility. Such EV depleted of membrane-tethered TGF-β could be advantageously loaded with a bioactive agent or cargo (e.g., polypeptides or polynucleotides (RNA, miRNA) and would not possess the biological activity of TGF-β. Other methods for EV sorting on the basis of membrane-tethered TGF-β would serve the same purpose. By way of example, TGF-β negative EV therapy may be more effective than EV with membrane-tethered TGF-β as treatment or therapy for particular diseases or conditions, such as pro-fibrotic states, e.g., the fibrotic phase after myocardial infarction.

In an embodiment, TGF-β can be removed from isolated EV having membrane-tethered TGF-β by enzymatic digestion, e.g., with proteases, glycanases, or heparinases. In an embodiment, EV having membrane tethered TGF-β of EV with membrane tethered EV removed may be loaded with a bioactive agent as described herein and employed as an immunogenic vector. By way of example, a dendritic cell-derived extracellular vesicles (EV) in which membrane-tethered TGF-β is removed can be genetically engineered to contain an antigen, e.g., protein or peptide, that is presented by the dendritic EV to immune cells can serve as a cancer vaccine which lacks immunosuppressive activity. Accordingly, the removal of membrane tethered TGF-β from EV may enhance the anti-tumor effectiveness of EV used as tumor vaccines, or may allow a reduced amount of such EV to be administered to a subject in need.

In another embodiment, the determination of disease status in a patient may be used to implement indirect therapy, aimed at augmenting endogenous membrane-tethered forms of TGF-β, without directly resupplying membrane-tethered TGF-β EV to the patient, for example, gene therapy, oligonucleotides (RNAi, antisense), nano-pharmaceuticals, artificial EV comprising excess membrane-tethered TGF-β, or inducers of downstream signals to TGF-β (e.g., SMADs, which are transcription factors that transduce extracellular TGF-β superfamily ligand signaling from membrane bound TGF-β receptors into the nucleus (following phosphorylation at their carboxy termini by the activated receptors where they activate the transcription of TGF-β target genes.).

In another embodiment, the surface membrane-expression of TGF-β may be enhanced or augmented in EV (e.g., derived from MSC) by pre-conditioning donor cells or cell lines, in particular, MSC, in culture (e.g. by treatment of the cell lines with hypoxia as described infra, or by exposure to inflammatory mediators such as IFNγ, TNFα, LPS, or IL-17, 5-50 ng/ml); by overexpressing tethered TGF-β using direct conjugation of TGF-β plus beta-glycan complexes; or by synthesis of artificial EV which are chemically decorated with a high density of tethered TGF-β. In a particular embodiment, EV derived from MSC are employed in the biomanufacture of MSC-EV with membrane-tethered TGF-β. In other embodiments, cells lines that are immune privileged, e.g., cells obtained from umbilical cord, placenta, fetal tissue, testes, etc., are also useful for production of membrane-tethered TGF-β EV. In another embodiment, the EV are treated with hyaluronidase to remove hyaluronic acid/proteoglycan complexes on their surfaces.

In an embodiment, the invention provides extracellular vesicles (EV) which express TGF-β or an isoform thereof tethered to the vesicle membrane, in which the TGF-β is recombinantly expressed and produced using the techniques of molecular biology as defined and described herein. In an embodiment, the EV comprising recombinant TGF-β tethered to the membrane are expressed in immune privileged cells, such as mesenchymal stromal cells or they are expressed in dendritic cells. By way of example, cells employed for EV production (e.g., MSC or immortalized MSC) can be transfected with a lentivirus or adeno-associated virus vector or transduced with a plasmid vector for selection and overexpression of encoded tethered TGF-β or a fusion protein (e.g., TGF-β fused to an EV membrane protein such as LAMP-2 or CD29). Following stable transduction, biogenesis of EV from transduced cells that overexpress TGF-β (or fusion protein) will increase the quantity of TGF-β that is decorated (expressed) on the surface of EV generated by those cell lines. Extracellular vesicles (EV) with membrane-tethered TGF-β produced and expressed in this way can have increased immunosuppressive effects which are therapeutically advantageous.

By way of example, a nonlimiting process for the production of extracellular vesicles (EV) comprising recombinant TGF-β tethered to the membrane can include the following: a) constructing, by conventional molecular biology methods, an expression vector that encodes and expresses TGF-β protein, e.g. TGF-β1, TGF-β2, TGF-β3, TGF-β4, or a combination thereof, thereby producing a vector for TGF-β expression; b) transferring or delivering the expression vector to a host cell by conventional molecular biology methods to produce a transfected host cell which expresses TGF-β in the membrane; and c) culturing the transfected (or transformed) host cell by conventional cell culture techniques so as to produce cells that produce and shed extracellular vesicles (EV) comprising recombinant TGF-β tethered to the membrane. The host cell used to express the recombinant TGF-β is preferably a eukaryotic cell (e.g., an MSC or another cell type, such as Chinese hamster ovary (CHO) cell). The choice of expression vector is dependent upon the choice of the host cell and may be selected so as to have the desired expression and regulatory characteristics in the selected host cell. In an embodiment, the host cell is a mesenchymal stromal cell (MSC), in particular, immortalized MSC.

In an embodiment, the amount of TGF-β tethered to the membrane of the extracellular vesicles (EV) may be modified, e.g., reduced or increased, by culturing mesenchymal stromal cells (MSC) expressing the EV having membrane tethered TGF-β in medium comprising or conditioned with certain additives, such as cytokines, factors, or agents, for example, interferon-gamma (IFN-β), tumor necrosis factor (TNF), interleukins, such as IL-17, or lipopolysaccharide (LPS), or by modifying the pH or microenvironment (e.g., employing 3D-cultures , spheroids, or similar structures instead of 2D cultures). In an embodiment, the enhancement of or increase in the amount of extracellular vesicles (EV) having membrane tethered TGF-β expressed by mesenchymal stromal cells (MSC) can be achieved by culturing the MSC under hypoxic or oxygen-glycose-deprived conditions. For example, MSC in culture medium are exposed to hypoxic conditions (e.g., 1-5% O₂ for 24 hours), are deprived of oxygen, or are deprived of oxygen and glucose for 1-12 hours at 37° C., 100% humidity, and 5% CO₂ Under such conditions, an increase of membrane-tethered TGF-β EV produced by MSC may be from about 1.5-fold to 4-fold relative to MSC not subject to such conditions. In embodiment, an increase of membrane-tethered TGF-β EV produced by MSC may be from least about or equal to 1.5-fold to 25-fold, or at least about or equal to 1.5-fold to 15-fold, or at least about or equal to 1.5-fold to 10-fold, or at least about or equal to 1.5-fold to 5-fold, including values therebetween relative to MSC not subject to such conditions.

In another embodiment, decreased levels of TGF-β tethered to the EV membrane can be achieved by altering gene expression (siRNA, miRNA or miRNA mimics, oligonucleotides) in the vesicles. In another embodiment, decreased levels of TGF-β tethered to EV can be achieved by disrupting the beta-glycan structure that tethers TGF-β to the membrane by exposing the membrane-tethered TGF-β EV to heparinases, betaglycanases (pervanadate), or other methods of enzymatic digestion, or by acid treatment. In another embodiment, mesenchymal stromal cells (MSC) that have been immortalized (e.g., using transduction with hTERT or SV40T) can be used to express and produce extracellular vesicles (EV) having increased levels of membrane-tethered TGF-β on EV as described supra (e.g., using plasmid transduction of TGF-β or TGF-(3-fusion protein).

In another embodiment, extracellular vesicles (EV) comprising TGF-β or an isoform thereof tethered to the membrane surface can be synthetically produced using techniques, e.g., phospholipid chemistry techniques, known in the art. For example, techniques routinely practiced in the art of liposome production may be used. (Alving, C. R., 1991, J. Immunol. Methods., 140:1-13; Wagner, A. et al., 2011, J. Drug Delivery, Vol. 2011, Article ID 591325, 9 pages). As would be appreciated by the skilled practitioner, liposomes are vesicles comprised of concentrically ordered phopsholipid bilayers, which encapsulate an aqueous phase. Liposomes typically comprise various types of lipids, phospholipids, and/or surfactants. The components of liposomes are arranged in a bilayer configuration, similar to the lipid arrangement of biological membranes (cell or extracellular vesicle (EV) membranes). Methods for preparation of liposomes are known in the art, for example, as provided by Epstein et al, 1985, Proc. Natl. Acad. Set USA, 82:3688; Hwang et al, 1980, Proc. Natl. Acad. Sci. USA, 77:4030-4; and U.S. Pat. Nos. 4,485,045 and 4,544,545. In addition, vesicle forming lipids can be used to formulate liposomes. Such lipids typically comprise with two hydrocarbon chains, such as acyl chains and a polar head group. Examples of vesicle forming lipids include phospholipids, e.g., phosphatidylcholine, phosphatidylethanolamine, phosphatidic acid, phosphatidylinositol, sphingomyelin and glycolipids, e.g., cerebrosides, gangliosides. In some embodiments, the liposomes or liposomal compositions further comprise a hydrophilic polymer, e.g., polyethylene glycol and ganglioside GM1, which increases the serum half-life of the liposome.

Also envisioned by the invention are sterically stabilized liposomes, which comprise membrane-tethered TGF-β and can be prepared using common methods known to the skilled practitioner. In general, sterically stabilized liposomes contain lipid components with bulky and highly flexible hydrophilic moieties that reduce the reaction of liposomes with serum proteins, reduce oposonization with serum components and reduce recognition by mononuclear phagocytic cells. Sterically stabilized liposomes can be prepared using polyethylene glycol. Liposomes and sterically stabilized liposomes can be prepared, for example, as reported in Bendas et al., 2001, BioDrugs, 15(4):215-224; Allen et al., 1987, FEBS Lett. 223:42-6; Klibanov et al, 1990, FEBS Lett, 268:235-7; Blum et al, 1990, Biochim. Biophys. Acta., 1029: 1-7; Torchilin et al, 1996, J. Liposome Res. 6:99-116; Litzinger et al, 1994, Biochim. Biophys. Acta, 1190:99-107; Maruyama et al, 1991, Chem. Pharm. Bull, 39:1620-2; Klibanov et al., 1991, Biochim Biophys Acta, 1062;142-8; Allen et al, 1994, Adv. Drug Deliv. Rev, 13:285-309. Liposomes that are adapted for specific organ targeting (U.S. Pat. No. 4,544,545), or specific cell targeting can also be used. Liposomes can be generated by a reverse phase evaporation method with a lipid composition comprising phosphatidylcholine, cholesterol, and PEG derivatized phosphatidylethanolamine (PEG-PE). Liposomes are extruded through filters of defined pore size to yield liposomes with the desired diameter. In an embodiment, artificial EV with membrane-tethered TGF-β can be synthesized as liposomes or exosome-liposome fusions in the size range (e.g., diameter) of EV and composed of phospholipids that closely resemble EV (e.g., ceramide, sphingomyelin). Such synthetic EV are decorated covalently with the TGF-β-beta-glycan complex (i.e., express a plurality of TGF-β proteins covalently attached to beta-glycans on the surface of the vesicles). In an embodiment, the expression of TGF-β on the EV membrane can be controlled using conventionally employed conditional expression systems, e.g., inducers or repressors that activate or inhibit, respectively, a response gene (e.g., TetON or TetOFF) that controls the promotor region of a gene transduced into a host cell, such as MSC. Fine tuning can be achieved by exposing the MSC or another cell line producing EV with specific levels of inducers or repressors, by methods practiced in the art, for example, Goverdhana, S. et al., 2005, Mol. Ther., 12(2):189-211.

In other embodiments, for therapeutic applications, MSC-derived EV with membrane-tethered TGF-β can be separated from the total EV population using immune affinity techniques, e.g., affinity chromatography (also called immune affinity capture herein), as described supra. By way of example, affinity chromatography can separate EV having membrane-tethered TGF-β based on the specificity of the interaction between TGF-β and a cognate molecule with which TGF-β has specificity, such as an anti-TGF-β antibody or a receptor ligand, e.g., through interactions such as hydrogen bonding, ionic interactions, disulfide bridges, hydrophobic interactions, etc. The high selectivity of affinity chromatography (e.g., immune affinity chromatography, which involves antibody-ligand binding interaction) results from the interaction of a desired molecule, e.g., TGF-β, with a specific ligand attached to the stationary phase, matrix, or medium of a chromatography column (e.g., a gel matrix which can be a polysaccharide polymer material typically derived from seaweed, e.g., agarose (a crosslinked, beaded form of agarose, e.g., Sepharose), such that the desired molecules becomes trapped within the column and can then be separated from non-specific or unwanted materials and components which do not interact (bind) to the ligand on the column and elute from the column in the mobile phase. The desired molecule, e.g., TGF-β, can be removed from the stationary phase by elution with an appropriate buffer solution, typically by changing the salt concentration, pH, pI, charge or ionic strength, as routinely practiced in the art. In addition to the foregoing, other types of affinity chromatography columns are also envisioned for use to separate and isolate extracellular vesicles (EV) having membrane-tethered TGF-β. In an embodiment, the MSC-derived EV with membrane-tethered TGF-β for therapeutic use are isolated from cell cultures of MSC or immortalized MSC as described herein.

In another embodiment, magnetic beads having bound anti-TGF-β (and/or antibodies to TGF-β isoforms, variants, signaling peptides, non-signaling peptides, or tethering proteins or side chains for example) can be used to separate EV having membrane-tethered TGF-β from the total population of EV in a sample. In an embodiment, EV with membrane-tethered TGF-β can be precipitated using magnetic columns (e.g. using AUTO-MACS®, Miltenyi Biotech, Inc., San Diego, Calif.). The purity of the selected EV having membrane-tethered TGF-β can be evaluated using methods described herein (e.g., nanoparticle tracking analysis (NTA), vesiculometry, or interferometry) or other methods that quantify or measure the abundance of EV with membrane-tethered TGF-β. Armed with knowledge of the quantity and purity of EV with membrane-tethered TGF-β, the skilled practitioner can derive and determine a dosage and formulation of TGF-β membrane-tethered EV for therapeutic purposes.

Results provided infra (see, e.g., Examples 2 and 3, infra) show that MSC-derived EV with membrane-tethered TGF-β have immunomodulatory effects on T cells, i.e., suppression of proliferation stimulated by mitogen. The invention provides methods for using MSC-derived EV with membrane tethered TGF-β, for example, those whose levels of membrane-tethered TGF-β have been manipulated so as to decrease or increase the levels of membrane-tethered TGF-β, for affecting changes in disease status or treatment, e.g., decrease immunosuppression of immune cell response or inhibition of cancer cell growth in vitro and in vivo. In an embodiment, EV comprising membrane-tethered TGF-β and derived from a given cell source, e.g., stromal cell, cancer-associated cells, mesenchymal stromal cells, can be engineered to contain and specifically deliver therapeutic agents for disease treatment or improved disease treatment, particularly, in vivo. In another embodiment, the EV comprising membrane-tethered TGF-β, e.g., MSC-derived, membrane-tethered TGF-β EV, can be engineered to express increased or decreased amounts of TGF-β in the membrane as described herein. In an embodiment, the EV are treated with hyaluronidase to remove hyaluronic acid/proteoglycan complexes on their surfaces.

Disease treatment and therapy e.g., treatment or therapy for autoimmune disease, inflammatory disease, cardiac disease, cancer, may be provided wherever disease treatment or therapy is performed, including a doctor's office, a clinic, a health or critical care facility, a hospital, a hospital's outpatient department, or at home. Treatment generally begins in a hospital or clinic so that the doctor or medical practitioner can observe the treatment's/therapy's effects closely and make any adjustments that are needed. The duration of the therapy depends on the kind of disease being treated, the age and condition of the patient, the stage and type of the patient's disease, and how the patient's body responds to the treatment. The administration of a treatment product or drug may be performed at different intervals (e.g., daily, weekly, or monthly) and may be repeated over time. For example, therapy may be given in on-and-off cycles that include rest periods so that the patient's body has a chance to build healthy new cells, exhibit a response and regain its strength.

Depending on the type of disease and its stage of development, the therapy can be used to reduce, abrogate, abate, diminish, ameliorate, or eliminate the disease or the symptoms or effects of the disease in a patient undergoing treatment. By way of example, the therapy can be used to slow the spreading of a cancer, to slow the cancer's growth, to kill or arrest cancer cells that may have spread to other parts of the body from the original tumor, to relieve symptoms caused by the cancer, or to prevent cancer in the first place. In addition, the therapy can be used to reduce the immunosuppression of immune cells involved in combatting the disease, to reduce inflammation, or to augment an immune response by immune cells. As described above, if desired, treatment with an agent, such as MSC-derived extracellular vesicles (EV) expressing membrane-tethered TGF-β as described herein, may be combined with conventional therapies, including therapies for the treatment of proliferative disease (e.g., disease-specific drugs and therapeutic compounds, radiotherapy, surgery, or chemotherapy). For any of the methods of application described above, an MSC-derived EV comprising membrane-tethered TGF-β of the invention is desirably administered intravenously or is applied to the site of neoplasia (e.g., by injection). Other modes of administration are also encompassed, including, without limitation, subcutaneous, intraperitoneal, intramuscular, intravaginal, intrathecal, bucal, rectal, intradermal, modes of administration.

In an embodiment, MSC-derived extracellular vesicles (EV) expressing membrane-tethered TGF-β can be administered or used to deliver therapeutic agents in vivo, without adverse reaction and without the development of a cellular inflammatory reaction. In a particular embodiment, MSC-derived EV comprising membrane-tethered TGF-β can be used as specific therapeutic agents. By way of example, MSC-derived EV comprising membrane-tethered TGF-β can be derived from immune privileged cells, i.e., those that do not elicit an inflammatory immune response (e.g., from immune privileged sites such as the umbilical cord, placenta, fetus, testes, articular cartilage), and can be administered to, or transplanted into, a subject having a disease and who is in need. MSC-derived EV comprising membrane-tethered TGF-β can contribute to modulation of cellular, e.g., immune cell, responses during disease. In addition, MSC-derived EV comprising membrane-tethered TGF-β can be used to exert an immunosuppressive effect in chronic inflammatory or auto-immune disease, or conditions leading to fibrosis, or those diseases and conditions that are preceded by inflammation (e.g., wound healing, arthritis, inflammatory bowel disease). In an embodiment, such MSC-derived EV comprising membrane-tethered TGF-β provide a therapeutic which has anti-immunosuppressive and anti-proliferative properties, as well as disease mitigating and survival-extending properties in vivo. In aspects of each of the above embodiments, the MSC-derived EV comprising membrane-tethered TGF-β can be loaded with one or more bioactive agents, e.g., a polypeptide, polynucleotide, or small molecule, as described herein below for use in disease treatment or therapy. In an embodiment, the EV are treated with hyaluronidase to remove hyaluronic acid/proteoglycan complexes.

In other aspects, the invention provides extracellular vesicles (EV) comprising membrane-tethered TGF-β, in particular, EV comprising membrane-tethered TGF-β isolated from mesenchymal stromal cells (MSC), for treating a disease, condition, pathology, or for diagnosing a disease, condition, or pathology (e.g., assessing or evaluating the status or progression of a disease, condition, or pathology, such as post-treatment or therapy monitoring), in which the disease is an autoimmune disease, transplant rejection, or other inflammatory disease, condition, or pathology. In accordance with aspects of the invention, MSC-derived extracellular vesicles (EV) expressing membrane-tethered TGF-β, or MSC-derived extracellular vesicles (EV) expressing modified (e.g., increased or reduced) amounts of membrane-tethered TGF-β, or MSC-derived extracellular vesicles (EV) expressing membrane-tethered TGF-β loaded with a bioactive agent, have utility in the treatment of inflammatory disease, autoimmune disease, or transplant rejection. In particular, such EV are capable of down-modulating the immune system of a subject, e.g., impairing the function or suppressing the proliferation and/or activity of immune cells such as CD4+ or CD8+ T cells, or other immune cells, e.g., natural killer (NK) cells, in vivo or in vitro. In another embodiment, the above-described MSC-derived membrane-tethered TGF-β EV have utility as a therapeutic in the treatment of various types of cancer.

Such down-modulation of the immune system and suppression of immune cell activity is typically desirable in the treatment and therapy of inflammatory and autoimmune diseases and in transplant rejection. Nonlimiting examples of autoimmune disorders that may be treated or managed by administering the extracellular vesicles (EV) comprising membrane-tethered TGF-β, particularly MSC-derived EV having membrane tethered TGF-β, of the present invention include alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Bechet's disease, bullous pemphigoid, cardiomyopathy, celiac sprue-dermatitis, chronic fatigue immune dysfunction syndrome, chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, neuromyelitis optica (NMO), type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, rheumatoid arthritis, sarcoidosis, scleroderma, Sjogrens' syndrome, stiff-man syndrome, systemic lupus erythematosus, lupus erythematosus, takayasu arteritis, temporal arteristis/giant cell arteritis, ulcerative colitis, uveitis, vasculitis, e.g., dermatitis herpetiformis vasculitis, vitiligo, and Wegener's granulomatosis.

Nonlimiting examples of inflammatory diseases that can be treated or managed with the extracellular vesicles (EV) comprising membrane-tethered TGF-β, particularly MSC-derived EV having membrane tethered TGF-β, of the present invention include, but are not limited to, asthma, encephalitis, inflammatory bowel disease (IBD), chronic obstructive pulmonary disease (COPD), allergic disorders, septic shock, pulmonary fibrosis, undifferentiated spondyloarthropathy, undifferentiated arthropathy, arthritis, inflammatory osteolysis, and chronic inflammation resulting from chronic viral infection or bacterial infection.

### Companion Diagnostics

In an aspect of the invention, TGF-β tethered to EV derived from a given cell type, e.g., a mesenchymal stromal cell, can be used as a biomarker of disease in a companion diagnostic method. As appreciated by the skilled practitioner, companion diagnostics are bioanalytical methods (diagnostic tests) designed to assess whether a patient having a disease will respond or has responded favorably to a specific medical treatment or therapy for the disease. A biomarker (e.g., levels or characteristics of a biomarker relative to those of a control) in a patient's biological sample undergoing testing is typically assessed in the companion diagnostic method. The linkage between the therapeutic treatment and biomarker levels could be important in the therapeutic application and clinical outcome of the use of a drug or therapeutic regimen in the patient (personalized medicine), or an important component of the drug development process. In addition, biomarker(s) used in the specific context of disease being treated provide(s) biological and/or clinical information that enables better decision making by the medical and clinical practitioner (and sometimes by the patient) about the course of present and future treatment of the patient's disease, as well as the development and use of other treatments or other potential drug therapy. The practice of a companion diagnostic method can be applied anywhere along the preclinical, clinical and post-product launch of a drug or therapy for a disease.

Accordingly, extracellular vesicles (EV) comprising membrane-tethered TGF-β can be used for diagnostic purposes, such as to detect, diagnose, or monitor diseases, disorders or infections. By way of example, the detection or diagnosis of a disease, disorder or infection, particularly an autoimmune disease comprises: (a) assaying the level of extracellular vesicles (EV) comprising membrane-tethered TGF-β in a biological sample obtained from a subject having a disease, disorder, or infection using one or more antibodies (or fragments thereof) that immunospecifically bind to the tethered TGF-β; and (b) comparing the level of the tethered TGF-β with a control level, e.g., levels in normal (non-diseased or healthy) subjects' samples, e.g., those who do not have, or who do not have detectable amounts of, membrane tethered-TGF-β EV, wherein an increase or decrease in the assayed level of tethered TGF-β compared to the control level of tethered TGF-β is indicative of the disease, disorder or infection. Such assays may include, without limitation, immunoassays, such as the enzyme linked immunosorbent assay (ELISA), radioimmunoassay (MA), fluorescence-activated cell sorting (FACS), and flow cytometry assays. In an embodiment, the EV are treated with hyaluronidase to remove hyaluronic acid/proteoglycan complexes. In another embodiment, the TGF-β tethered to the EV is in a latent form.

Patient status in response to therapy comprising MSC-derived membrane-tethered TGF-β EV (or other therapies) can be determined and monitored in a biofluid sample obtained from a subject using EV with tethered TGF-β (including TGF-β isoforms, mutant or variant forms of TGF-β, latency versus active forms of TGF-β, or quantified in relation to more traditional markers), thus constituting a companion diagnostic. Thus, the level or amount of endogenous EV having membrane tethered TGF-β as detected by the methods described herein can guide the use of MSC-derived EV with membrane-tethered TGF-β as a therapeutic. For patient screening or monitoring, EV expressing tethered TGF-β in biological samples can be quantified, and/or these EV can be analyzed further for the presence of particular isoforms or variant forms of TGF-β (or secondary molecules as markers or molecular signatures) in a test population, and can be compared, for example, to a control which is a sample having tethered TGF-β negative (or low expressing) EV, which can be used to refine the diagnostic accuracy of the findings. Also, the observed differences in types or forms of the tethered TGF-β in patients identified as having high levels of TGF-β tethered to EV versus those in patients identified as having low/negative levels of TGF-β tethered to EV may be exploited as a biomarker or signature of patient status leading to decisions on disease treatment and management in patients. Examples of the abovementioned secondary molecular signatures include RNA species, DNA, bioactive lipids, proteins, and metabolites such as adenosine. These biomarkers can also be assessed in patient populations to evaluate safety, activity, efficacy, or clinical effectiveness of an intervention in a clinical setting.

In some instances, MSC-derived EV with membrane-tethered TGF-β may be employed as a treatment agent or therapeutic without guidance from patient status (endogenous EV with membrane-tethered TGF-β), for example, to exert an immunosuppressive effect in chronic inflammatory or autoimmune disease, or in conditions leading to fibrosis, or those that are preceded by inflammation (e.g. wound healing). In other instances, patient status can be employed to understand the immunologic status of the patient without the use of specific therapy, such as therapy involving MSC derived EV with membrane-tethered TGF-β.

### TGF-β Tethered EV Containing Proteins, Polypeptides, or Peptides

The EV comprising membrane-tethered TGF-β, in particular, MSC-derived EV expressing membrane-tethered TGF-β as described herein, may contain proteins, polypeptides, or peptides, particularly for therapeutic or treatment purposes as described supra. In an embodiment, the EV are treated with hyaluronidase to remove hyaluronic acid/proteoglycan complexes. In a specific embodiment, the EV expressing membrane-tethered TGF-β can contain (and deliver to a cell or tissue, or to a cell or tissue in a subject) an agent, e.g., a protein, that increases or decreases an immune response by (an) immune cell(s), corrects a deficiency of the cell or subject, or induces the death of infected or deficient cells. Recombinant polypeptides are produced using virtually any method known to the skilled practitioner. Typically, recombinant polypeptides are produced by transformation of a suitable host cell with all or part of a polypeptide-encoding nucleic acid molecule or fragment thereof in a suitable expression vehicle.

Those skilled in the field of molecular biology will appreciate that any of a wide variety of expression systems may be used to provide a recombinant protein. The precise host cell used is not critical to the invention. A polypeptide for use may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells or CHO cells). Such cells are available from a wide range of sources (e.g., the American Type Culture Collection, Manassas, Md.; also, see, e.g., Ausubel et al., Current Protocols in Molecular Biology, New York: John Wiley and Sons, 1997). The method of transformation or transfection and the choice of expression vehicle will depend on the host system selected. Transformation and transfection methods are described, e.g., in Ausubel et al., supra; expression vehicles can be from among those provided, e.g., in Cloning Vectors: A Laboratory Manual (P. H. Pouwels et al., 1985, Supp. 1987).

A variety of expression systems exist for the production of the polypeptides that can be used in conjunction with the membrane-tethered TGF-β EV described herein. Membrane-tethered TGF-β EV derived from a given cell type, e.g., MSC or fibroblast-like cells, can be loaded with one or more expression vectors or with the polypeptides generated using such vectors. Nonlimiting examples of expression vectors useful for producing polypeptides include chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

A particular bacterial expression system for polypeptide production is the E. coli pET expression system (e.g., pET-28) (Novagen, Inc., Madison, Wis.). In this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically accomplished using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids can be cleaved with thrombin; those expressed in pGEX-3X plasmids can be cleaved with Factor Xa.

Alternatively, recombinant polypeptides may be expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is encoded by the AOX1 gene is induced by methanol. The AOX1 promoter can be used for inducible polypeptide expression or the GAP promoter for constitutive expression of a gene of interest.

Once a given recombinant polypeptide is expressed, it is isolated, for example, using affinity chromatography. In one example, an antibody raised against the polypeptide may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified by methods known and practiced in the art, e.g., by high performance liquid chromatography (see, e.g., Fisher, Laboratory Techniques In Biochemistry and Molecular Biology, eds., Work and Burdon, Elsevier, 1980). Polypeptides, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs.

The isolated polypeptides or fragments can be loaded into TGF-β tethered EV using methods practiced in the art.

### TGF-β Tethered EV Containing Polynucleotides

The EV comprising membrane-tethered TGF-β, in particular, MSC-derived EV expressing membrane-tethered TGF-β as described herein may contain one or more polynucleotides, particularly for therapeutic or treatment purposes as described supra. In a specific embodiment, the EV expressing membrane-tethered TGF-β can contain (and deliver in a subject) a polynucleotide, that corrects a deficiency of the cell or subject, or induces the death of infected or deficient cells. In an embodiment, the polynucleotide encodes a protein product that corrects a deficiency of the cell or subject, or induces the death of infected or deficient cells. Nonlimiting examples of polynucleotides include RNA, DNA, an antisense oligonucleotide, a short interfering RNA (siRNA), a short hairpin RNA (shRNA), plasmid DNA polynucleotides and modified oligonucleotides.

Membrane-tethered TGF-β EV may also be molecularly engineered to contain expression vectors harboring a polynucleotide with therapeutic function. In an embodiment, MSC-derived membrane-tethered TGF-β EV may be administered to a subject having a disease, e.g., inflammation or autoimmune disease or cancer, for delivery to the subject's cells. In an embodiment, the DNA encodes a protein with a specific diagnostic or therapeutic function. Membrane-tethered TGF-β EV, particularly, MSC-derived membrane-tethered TGF-β EV, comprising nucleic acid molecules are selectively delivered to target cells of a subject (e.g., cancer cells) in a form in which they are taken up and are advantageously expressed so that therapeutically effective levels can be achieved.

An isolated nucleic acid molecule can be manipulated using recombinant DNA techniques well known in the art. Thus, a nucleotide sequence contained in a vector in which 5′ and 3′ restriction sites are known, or for which polymerase chain reaction (PCR) primer sequences have been disclosed, is considered isolated, but a nucleic acid sequence existing in its native state in its natural host is not. An isolated nucleic acid may be substantially purified, but need not be. For example, a nucleic acid molecule that is isolated within a cloning or expression vector may comprise only a small percentage of the material in the cell in which it resides. Such a nucleic acid is isolated, however, as the term is used herein, because it can be manipulated using standard techniques known to those of ordinary skill in the art.

Transducing viral (e.g., retroviral, adenoviral, lentiviral and adeno-associated viral) vectors can be used to deliver polynucleotides to/into cells (as well as TGF-β tethered EV) and for somatic cell gene therapy, especially because of their high efficiency of infection and stable integration and expression (see, e.g., Cayouette et al., Human Gene Therapy, 8:423-430, 1997; Kido et al., Current Eye Research, 15:833-844, 1996; Bloomer et al., J. Virology, 71:6641-6649, 1997; Naldini et al., Science, 272:263-267, 1996; and Miyoshi et al., Proc. Natl. Acad. Sci. U.S.A., 94:10319, 1997). By way of nonlimiting example, a polynucleotide can be cloned into a retroviral or other vector and expression can be driven from its endogenous promoter, from the retroviral long terminal repeat, or from a promoter specific for a target cell type of interest. Other useful viral vectors include, for example, a vaccinia virus, a bovine papilloma virus, or a herpes virus, such as Epstein-Barr Virus (also see, for example, the vectors reported by Miller, Human Gene Therapy, 15-14, 1990; Friedman, Science, 244:1275-1281, 1989; Eglitis et al., BioTechniques, 6:608-614, 1988; Tolstoshev et al., Current Opinion in Biotechnology, 1:55-61, 1990; Sharp, The Lancet, 337:1277-1278, 1991; Cornetta et al., Nucleic Acid Research and Molecular Biology, 36:311-322, 1987; Anderson, Science, 226:401-409, 1984; Moen, Blood Cells, 17:407-416, 1991; Miller et al., Biotechnology, 7:980-990, 1989; Le Gal La Salle et al., Science, 259:988-990, 1993; and Johnson, Chest, 107:77S-83S, 1995). Retroviral vectors are particularly well developed and have been used in clinical settings (Rosenberg et al., N. Engl. J. Med., 323:370, 1990; Anderson et al., U.S. Pat. No.5,399,346).

Polynucleotide expression can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. Such enhancers used can include, without limitation, those that are characterized as tissue- or cell-specific enhancers.

MSC-derived membrane-tethered TGF-β EV can contain and deliver nucleic acid molecules comprising a modified nucleic acid. Nucleic acid molecules include nucleobase oligomers containing modified backbones or non-natural internucleoside linkages. Oligomers having modified backbones include those that retain a phosphorus atom in the backbone and those that do not have a phosphorus atom in the backbone. For the purposes of this specification, modified oligonucleotides that do not have a phosphorus atom in their internucleoside backbone are also considered to be nucleobase oligomers. Nucleobase oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included. Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Nucleobase oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or by one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts. Representative United States patents that teach the preparation of the above oligonucleotides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Nucleobase oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleobase oligomer, particularly the 3′ position of the sugar on the 3′ terminal nucleotide. Nucleobase oligomers may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

In other nucleobase oligomers, both the sugar and the internucleoside linkage, i.e., the backbone, are replaced with novel groups. Methods for making and using these nucleobase oligomers are described, for example, in Peptide Nucleic Acids (PNA): Protocols and Applications, Ed. P. E. Nielsen, Horizon Press, Norfolk, United Kingdom, 1999. Representative United States patents that teach the preparation of PNAs include, but are not limited to, U.S. Pat. Nos. 5,539,082; 5,714,331; and 5,719,262, each of which is herein incorporated by reference. PNA compounds are also reported by Nielsen et al., Science, 1991, 254, 1497-1500.

### TGF-β Tethered EV and Imaging Agents

The EV comprising membrane-tethered TGF-β as described herein may contain a detectable agent useful for imaging studies. The invention provides TGF-β tethered EV comprising any one of the following exemplary small molecules useful in imaging: carbocyanine, indocarbocyanine, oxacarbocyanine, thilicarbocyanine and merocyanine, polymethine, coumarine, rhodamine, xanthene, fluorescein, borondipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800R5, IRDye 700DX, ADS780WS, ADS830WS, and ADS832WS.

In other embodiments, the TGF-β tethered EV comprise a nanoparticle useful in imaging studies. In one embodiment, nanoparticles are synthesized using a biodegradable shell known in the art. In one embodiment, a polymer, such as poly (lactic-acid) (PLA) or poly (lactic-co-glycolic acid) (PLGA) is used. Such polymers are biocompatible and biodegradable, and are subject to modifications that desirably increase the circulation lifetime of the nanoparticle. In one embodiment, nanoparticles are modified with polyethylene glycol (PEG), which increases the half-life and stability of the particles in circulation (Gref et al., Science, 263(5153): 1600-1603, 1994). In an embodiment, the EV are treated with hyaluronidase to remove hyaluronic acid/proteoglycan complexes.

Biocompatible polymers useful in the compositions and methods described herein include, but are not limited to, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetage phthalate, carboxylethyl cellulose, cellulose triacetate, cellulose sulfate sodium salt, poly(methylmethacrylate), poly(ethylmethacrylate), poly(butylmethacrylate), poly(isobutylmethacrylate), poly(hexlmethacrylate), poly(isodecylmethacrylate), poly(laurylmethacrylate), poly(phenylmethacrylate), poly(methyl acrylate), poly(isopropylacrylate), poly(isobutylacrylate), poly(octadecylacrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, polyhyaluronic acids, casein, gelatin, gluten, polyanhydrides, polyacrylic acid, alginate, chitosan, poly(methyl methacrylates), poly(ethylmethacrylates), poly(butylmethacrylate), poly(isobutylmethacrylate), poly(hexlmethacrylate), poly(isodecl methacrylate), poly(lauryl methacrylate), poly(phenyl methacrylate), poly(methyl acrylate), poly(isopropyl acrylate), poly(isobutyl acrylate), poly(octadecl acrylate) and combinations of any of these. In one embodiment, the nanoparticles of the invention include PEG-PLGA polymers.

In response to the growing need for encapsulation materials, several different approaches to producing hollow polymeric capsules are available. In one example, the shell is composed of dendrimers (Zhao, M., et al., 1998, J Am. Chem. Soc., 120:4877). A dendrimer is an artificially manufactured or synthesized large molecule comprised of many smaller ones linked together—built up from branched units called monomers. Technically, dendrimers are a unique class of a polymer, about the size of an average protein, with a compact, tree-like molecular structure, which provides a high degree of surface functionality and versatility. Their shape gives them vast amounts of surface area, making them useful building blocks and carrier molecules at the nanoscale level; they are available in a variety of forms, with different physical (including optical, electrical and chemical) properties. In other embodiments, the shell comprises block copolymers (Thurmond, K. B., II, et al., 1997, J. Am. Chem. Soc., 119:6656; MacKnight, W. J., et al., 1998, Acc. Chem. Res., 31:781; Harada, A. and Kataoka, K., 1999, Science, 283:65), vesicles (Discher, B. M., et al., 1999, Science, 284:1143), hydrogels (Kataoka, K. et al., 1998, J. Am. Chem. Soc., 120:12694) and template-synthesized microtubules (Martin, C. R. and Parthasarathy, R. V., 1995, Adv. Mater., 7:487) that are capable of encapsulating a photosensitizer.

In another embodiment, a TGF-β tethered EV of the invention comprises an isotopic label for positron or scintillation or SPECT imaging. In another embodiment, a TGF-β tethered EV of the invention comprises a magnetic nanoparticle that has a high magnetic moment to enhance the selectivity of the nanoparticle for detection. In another embodiment, a magnetic nanoparticle includes a magnetic core and a biocompatible outer shell, in which the outer shell both protects the core from oxidation and enhances magnetic properties of the nanoparticle. The enhanced magnetic properties can include increased magnetization and reduced coercivity of the magnetic core, allowing for highly sensitive detection as well as diminished non-specific aggregation of nanoparticles. By forming biocompatible nanoparticles having enhanced magnetic properties, detection of specific target proteins and cells is provided. In one embodiment, a nanoparticle core is formed from ferromagnetic materials that are crystalline, poly-crystalline, or amorphous in structure. For example, the nanoparticle core can include materials such as, but not limited to, Fe, Co, Ni, FeOFe₂O₃, Ni O Fe₂ O₃, CUOFe₂ O₃, MgOFe₂ O₃, MnBi, MnSb, MnOFe₂0₃, Y3Fe₅ O i₂, Cr O₂, MnAs, SmCo, FePt, or combinations thereof.

In another embodiment, the outer shell of the magnetic nanoparticle partially or entirely surrounds the nanoparticle core. In some implementations, the shell is formed from a superparamagnetic material that is crystalline, poly-crystalline, or amorphous in structure. In some cases, the material used to form the shell is biocompatible, i.e., the shell material elicits little or no adverse biological/immune response in a given organism and/or is nontoxic to cells and organs. Exemplary materials that can be used for the shell include, but are not limited to, metal oxides, e.g., ferrite (Fe₃C″4), FeO, Fe203, CoFe₂04, MnFe₂04, NiFe₂04, ZnMnFe₂04, or combinations thereof.

Methods of making and delivering nanoparticles are known in the art and described, for example, in the following US Patent Publications: 20150258222, 20140303022, 20130309170, and 20130195767.

### TGF-β Tethered Extracellular Vesicle (EV) Isolation, Loading, and Targeting

TGF-β tethered EV as described herein are generated as described herein below. In general, the EV expressing membrane tethered TGF-β are released by cells (e.g., stromal cells, stromal-stem cells, mesenchymal stromal cells, cancer-associated fibroblasts, fibroblast-like cells) into the extracellular environment. TGF-β tethered EV can be isolated from a variety of biological fluids (biofluids), including, but not limited to, blood, plasma, serum, saliva, sputum, urine, stool, semen, cerebrospinal fluid, prostate fluid, lymphatic drainage, bile fluid, and pancreatic secretions. The TGF-β tethered EV can be separated or isolated using routine methods known in the art. In an embodiment, TGF-β tethered EV are isolated from the supernatants of cultured cells using differential ultracentrifugation. In another embodiment, TGF-β tethered EV are separated from nonmembranous particles, using their relatively low buoyant density (Raposo, G. et al., 1996, JEM, 183(3):1161; Raposo, G. et al., 2013, J. Cell Biol., 200(4):373-383); Escola, J. M. et al., 1998, J. Biol. Chem., 273(32):20121-7; van Niel, G. et al., 2003, Gut, 52(12):1690-7); Wubbolts, R. et al., 2003, J. Biol. Chem., 278:10963-10972). Kits for such isolation are commercially available, for example, from Qiagen, InVitrogen and SBI.

Methods for loading EV, in particular, TGF-β tethered EV, with an agent of interest, such as a bioactive agent: polypeptide or polynucleotide (cargo), are known in the art and include lipofection, electroporation, calcium chloride precipitation, as well as any standard transfection method.

In one embodiment, the TGF-β tethered EV comprising a polynucleotide or polypeptide, or small molecule of interest are obtained by over-expressing the polynucleotide or polypeptide or loading the cells with the small molecule in culture and subsequently isolating indirectly modified TGF-β tethered EV from the cultured cells. In another embodiment, TGF-β tethered EV comprising a polynucleotide or polypeptide or small molecule of interest are generated by loading previously purified TGF-β tethered EV with the molecule(s) of interest into/onto the TGF-β tethered EV by electroporation (polynucleotide or polypeptide), covalent or non-covalent coupling to the EV surface (polynucleotide or polypeptide or small molecule) or simple co-incubation (polynucleotide or polypeptide or small molecule).

### Pharmaceutical Compositions

Provided in another aspect are MSC-derived membrane-tethered TGF-β EV for as a therapeutic and MSC-derived membrane-tethered TGF-β EV for delivering an agent (e.g., a bioactive agent such as a polynucleotide, polypeptide, or small molecule) for the treatment of disease. In an embodiment, the present invention provides a pharmaceutical composition comprising MSC-derived membrane-tethered TGF-β EV as a therapeutic. In another embodiment, a pharmaceutical composition comprising MSC-derived membrane-tethered TGF-β EV for delivery of an agent (e.g., polynucleotide, polypeptide, or small molecule) is provided. In embodiments, the TGF-β tethered EV is derived from a stromal cell, a stromal stem cell, a mesenchymal stromal cell (MSC), a cancer-associated fibroblast, or a fibroblast-like cell. In a particular embodiment, the EV expressing membrane tethered TGF-β is derived from MSC. The MSC-derived membrane-tethered TGF-β EV of the invention may be administered as part of a pharmaceutical composition. In general, the MSC-derived membrane-tethered TGF-β EV are provided in a physiologically balanced saline solution. The solution comprising the MSC-derived membrane-tethered TGF-β EV may be stored at room temperature for up to about 24 hours, for longer than twenty four hours; such solutions can also be stored at about 4° C. for days, weeks, or months. MSC-derived membrane-tethered TGF-β EV may be frozen for long term storage, e.g., for up to 10 years. The compositions should be sterile and contain a therapeutically effective amount of the MSC-derived membrane-tethered TGF-β EV in a unit of weight or volume suitable for administration to a subject.

MSC-derived membrane-tethered TGF-β EV of the invention may be administered within a pharmaceutically-acceptable diluent, carrier, or excipient, in unit dosage form. Conventional pharmaceutical practice may be employed to provide suitable formulations or compositions to administer the compounds to patients suffering from a disease (e.g., cardiac disease, cancer). Administration may begin before the patient is symptomatic.

Any appropriate route of administration may be employed. The methods of the invention, generally speaking, may be practiced using any mode of administration that is medically acceptable, i.e., any mode that produces effective levels of the TGF-β tethered EV (as active) without causing clinically unacceptable, adverse effects. By way of nonlimiting example, modes and routes of administration may include parenteral, bucal, intravenous, intra-arterial, subcutaneous, intratumoral, intramuscular, intracranial, intracerebroventricular, intraorbital, ophthalmic, intraventricular, intrahepatic, intracapsular, intrathecal, intracisternal, intraperitoneal, intranasal, intratracheal, aerosol, topical, transdermal, intravaginal, rectal (suppository), oral administration, or within/on implants, e.g., fibers such as collagen, osmotic pumps, or tissue or synthetic grafts comprising appropriately transformed cells, etc. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000, and updates thereof. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for the TGF-β tethered EV include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a disease or condition. The preferred dosage of a TGF-β tethered EV of the invention is likely to depend on such variables as the type and extent of the disease or disorder, the overall health status and condition of the particular patient, the formulation of the excipients, and its route of administration.

With respect to a subject having a neoplastic disease or disorder, an effective amount is sufficient to stabilize, slow, or reduce the proliferation of the neoplasm. Generally, doses of TGF-β tethered EV or compositions thereof of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of TGF-β tethered EV.

### Kits

Kits provided by the invention include MSC-derived membrane-tethered TGF-β EV or a composition thereof; or MSC-derived membrane-tethered TGF-β EV containing an agent formulated for delivery to a cell in vitro or in vivo, or a composition thereof. In an embodiment, a kit contains MSC-derived EV that have been modified to contain a reduced level of TGF-β tethered to the membrane or MSC-derived EV that have been modified so as to remove TGF-β tethered to the membrane as described supra. Optionally, the kit includes directions for administering or delivering the MSC-derived membrane-tethered TGF-β EV (or modified EV) to a subject. In other embodiments, the kit comprises a sterile container which contains the MSC-derived membrane-tethered TGF-β EV or composition thereof; such containers can be boxes, ampules, bottles, vials, tubes, bags, pouches, blister-packs, or other suitable container forms known in the art. Such containers can be made of plastic, glass, laminated paper, metal foil, or other materials suitable for holding the MSC-derived membrane-tethered TGF-β EV or a composition thereof. The instructions will generally include information about the use of the MSC-derived membrane-tethered TGF-β EV. In other embodiments, the instructions include at least one of the following: description of the MSC-derived membrane-tethered TGF-β EV; methods for using the enclosed materials for the treatment of a disease; precautions; warnings; indications; clinical or research studies; and/or references. The instructions may be printed directly on the container (when present), or as a label applied to the container, or as a separate sheet, pamphlet, card, or folder supplied in or with the container.

It is believed that one skilled in the art, using the preceding description, can utilize the present invention to the fullest extent. The following examples are illustrative only, and are not intended to limit the remainder of the disclosure in any way.

### EXAMPLES

The following examples are set forth so as to provide those of ordinary skill in the art with a complete disclosure and description of how the compounds, compositions, articles, devices, and/or methods described and claimed herein are made and evaluated, and are intended to be purely illustrative and not to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Isolation of Immunomodulatory Extracellular Vesicles (EV) with Tethered TGF-β

In accordance with the present invention, the measurement of TGF-β tethered to the membrane of extracellular vesicles (EV) derived from a variety of cell, tissue, or organ types can be used for the assessment of the immune status of a subject, e.g., human and veterinary subjects, in need. The accurate quantification of TGF-β (or other immunomodulatory proteins) tethered to EV in subjects' biofluids provides an improved index of disease activity, aggressiveness, prognosis, and/or response to therapy, as well as other aspects related to the natural history of a subject's disease (e.g., status at a given time, progression, remission, regression, refraction, and the like) as described hereinabove.

Biofluid samples, such as blood, urine, cerebrospinal fluid, or saliva, obtained from patients, or cell culture supernatants, were cleared of cells, platelets, apoptotic bodies, cell debris, protein aggregates, and other particulates that were not extracellular vesicles (EV). This was achieved by differential centrifugation (1300×g for 10 minutes to remove cells and platelets; 2000×g for 10 minutes to remove apoptotic bodies; and 10,000×g for 30 minutes to remove microvesicles), or by sequential filtration after clarification of cells and apoptotic bodies using a 200 nm filter. For cell culture supernatants, initial concentration of EV was carried out by filtration to remove cells and cell debris (200 nm pore size), followed by tangential flow filtration (50,000-300,000 kDa molecular weight cutoff).

Extracellular vesicles (EV) were isolated from the clarified sample (e.g. plasma, serum, cell culture supernatant) by either affinity column, tangential flow filtration (e.g., >50 kDa molecular weight cut off filter), precipitation (e.g., using PEG, ExoQuik), differential ultracentrifugation (e.g., 100,000×g for 70 minutes using a 70Ti rotor to sediment EV), density gradient centrifugation, or size exclusion chromatography (e.g., 30-45 nm pore size). Other conventionally used methods for isolation and concentration of EV can also be used. Alternatively EV were isolated from clarified samples using a non-affinity (i.e., negatively charged like EV which have negative zeta potential) spin column that retained EV and could be washed to remove non-EV constituents without the loss of EV in the column.

The fraction of EV expressing TGF-β (including isoforms TGF-β1, TGF-β2, TGF-β3, and/or TGF-β4) on the surface, i.e. tethered to the EV membrane via beta glycan, (also referred to as TGF-βR3), was measured using a number of quantitative methods. Such methods include single vesicle nanoparticle tracking analysis by immunolabeling (e.g., fluorescent labeling) of TGF-β by QDOT®—(ThermoFisher Scientific, Waltham, Mass.) conjugated antibody (anti-TGF-β antibody) or by indirect labeling (e.g. biotin-conjugated antibody, streptavidin-QDOT) (Thane, K. E. et al., 2017, J. Extracell. Vesicles, in review); vesiculometry employing fluorescence detection of immunolabeled EV (Enjeti, A. K., et al., 2016, Thromb. Res., V. 145:18-23) or EV absorbed to beads; or interferometry (Daaboul, G. G., et al., 2016, Sci. Rep., Vol. 6:37246), as described hereinabove. As will be appreciated by the skilled practitioner, QDOT nanocrystal labelled-antibody conjugates provide both single and multicolor, multiplexed fluorescence detection using an excitation source, such as a 405 nm violet laser, particularly for low abundance molecules (antigens) with minimal photobleaching.

The above methods (and other suitable methods) assess or benchmark the quantity, phenotype and size distribution of EV with membrane-tethered TGF-β as biomarker. Data are quantified relative to total EV, total protein, or total EV-expressed proteins (e.g., CD9, CD63, CD81, TSG101, flotillin, synectin, LAMP-2, Alix), nucleic acids, lipids, or other constituents that represent the total EV population in a sample. The biomarker data were used to stratify patient status by stage, aggressiveness, prognosis, resistance to therapy, or any aspect of disease status. Stratification may involve (1) measuring membrane-tethered TGF-β on EV obtained from patients, (2) monitoring the levels (high and low) of TGF-β tethered to EV obtained from patients at different time points; or (3) treating patients presenting with high versus low TGF-β tethered to EV with different therapies, treatment regimens, monitoring schedules, drugs, adjuvant treatments, etc., for example.

### Example 2

### TGF-β Tethered to Exosome Extracellular Vesicles from Mesenchymal Stem-Stromal Cells (MSC) Suppress T-Helper Cell Division

Mesenchymal stem-stromal cells (MSC) suppress activation and proliferation of CD4+ T cells, and soluble transforming growth factor beta (β), (TGF-β) plays an important role in that mechanism. Immune suppression by membrane bound TGF-β is recognized in a dendritic cell and cancer associated fibroblast extracellular vesicles (EV), but this mechanism has not been documented for MSC-EV. It was hypothesized that EV membrane bound TGF-β (i.e., membrane-tethered TGF-β) is central to the immunomodulatory mechanism of MSC.

Serum-free culture medium from canine Wharton's Jelly mesenchymal stem cells (WJ-MSC: CD44⁺, CD90⁺, CD34⁻, CD45⁻, MHCII⁻, n=6 cell lines) was collected after 48 hours, and extracellular vesicles (WJ-EV) were isolated by differential centrifugation. WJ-EV output was assessed using single vesicle nanoparticle tracking analysis (NTA). CFSE-stained peripheral blood mononuclear cells (PBMC) were collected from healthy dogs (n=8), exposed to Concanavalin A mitogen (ConA; 5 μg/ml) and co-incubated with WJ-MSC (1:10) across transwell membrane (0.4 μm pore size) or with WJ-MSC EV (1:10⁴)±10 μM SB431542 (TGFβR1 inhibitor) or TGF-β neutralizing (e.g., inhibiting) antibody (Ab) for 72 hours. Analysis of CFSE fluorescence using FlowJo (v7.6.5) yielded % CD4⁺ cells that had undergone division (T-cell proliferation).

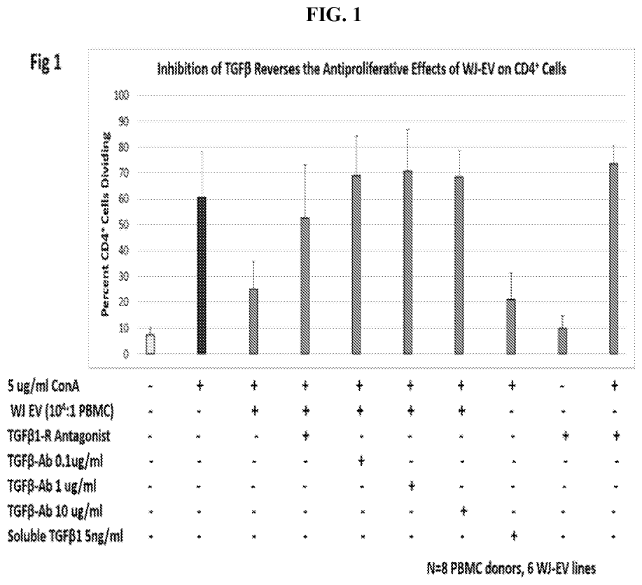

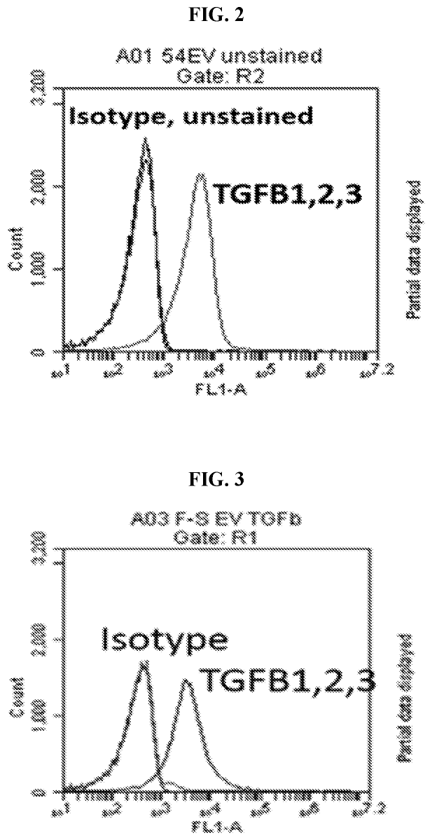

An average of 83±2% of the particle count from WJ-MSC conditioned medium were in the exosome size range (30-200 nm) based on NTA. The % CD4⁺ division in response to ConA alone (60±17%) was significantly higher than that observed in ConA+WJ-MSC (25±11%, P<0.01), ConA+WJ-EV (23±13%, P<0.01), or soluble TGF-β1 alone (21±10%, P<0.01). The addition of the TGFβR1 inhibitor SB431542 to ConA+WJ-EV increased CD4³⁰ division to 52±17% (P<0.01 vs ConA+WJ-EV). The addition of TGF-β Ab to ConA+WJ-EV at 0.1, 1, or 10 μg/ml resulted in CD4⁺ division of 58±14%, 60±16% and 58±10% (P<0.01 vs ConA+WJ-EV), respectively. (See, e.g., FIG. 1). In FIG. 1, it can be observed that mitogen (Concanavalin A) induced a striking increase in the percentage of CD4⁺ T cells, which proliferate in vitro. MSC and EV derived from MSV co-cultured with PBMC (including T cells) blocked this effect by 60-80%. The effect of TGF-β tethered to EV is similar to that of soluble TGF-β (FIG. 1). That TGF-β was tethered to the EV membrane surface was confirmed by bead-assisted flow cytometry (FIG. 2). FIG. 3 shows that TGF-β is also tethered to plasma EV (n=2 canine pooled samples), thus implying that certain EV are released from cells with varying amounts of surface-bound TGF-β and have the potential to dampen the immune response of cells within a variety of organs.

The experimental data in this Example demonstrate that mitogen-induced T-cell proliferation, which is markedly suppressed by WJ-EV, is mediated in part by membrane-bound TGF-β. The suppression of T cell proliferation by EV isolated from MSC (WJ-EV) is antagonized by a TGFβR1 inhibitor or TGF-β neutralizing antibody. It is possible to measure TGF-β tethered to EV alone, or in relation to other cytokines, e.g., IL-6, as a biomarker of various diseases and conditions.

### Example 3

### Canine Wharton's Jelly Mesenchymal Stem Cells (WJ-MSC) Regulate T Helper Cell Suppression Using Extracellular Vesicle Associated Transforming Growth Factor Beta (TGF-β) and Adenosine

Wharton's Jelly has emerged as a source of mesenchymal stem cells (MSC) in regenerative medicine. Wharton's Jelly MSC (WJ-MSC) are readily isolated from multiple regions of the umbilical cord, yielding greater numbers of MSC per gram of tissue than fat or bone marrow, for extended periods after discard and from cords harvested at multiple stages in gestation. WJ-MSC derived from extra-embryonic fetal tissue exhibit ‘youthful’ properties, such as Oct4 and Nanog expression, over several passages. This is in contrast to bone marrow MSC which demonstrate substantial donor age effects that reduce colony formation, cell expansion, and differentiation potential.

The immunomodulatory capacity of WJ-MSC has served as a rationale for the development of WJ-MSC for cell therapy (M. Rizk et al., 2017, Biol. Blood Marrow Transplant., 23(10):1607-1613). Some studies have reported that WJ-MSC exhibit comparable or superior immunomodulatory potential to that of adipose tissue derived MSC (AT-MSC) and bone marrow MSC (BM-MSC). WJ-MSC have also been reported to be less immunogenic than MSC from other sources (R. N. Barcia et al., 2017, Cytotherapy, 19(3):360-370).

A wide range of immunologic process are mitigated by WJ-MSC, including, but not limited to, suppression of T cell proliferation, promotion of a T regulatory cell phenotype, and polarization of macrophages toward an anti-inflammatory M2 phenotype in vitro. In one report, WJ-MSC failed to mitigate NK or B cell activation (Ribeiro, A. et al., 2013, Stem Cell Res Ther, 4(5):125), suggesting that monocytes and T cells are major targets. Immune modulation has been observed with or without contact between MSC and immune effector cells. In studies, contact between WJ-MSC and lymphocytes has shown either increased or decreased biological activity. Hence, MSC-immune effector cell interactions are complex and include reciprocal processes that may impact either cell type positively or negatively. The adverse effects of immune effector cells on MSC has ignited interest in their secretome, and a search for acellular MSC based products that may be more stable in the host microenvironment.

Within the MSC secretome are extracellular vesicles (EV), nanoscale cellular products that contain RNA, protein, and lipids that recapitulate many biological properties previously attributed to parent cells or their soluble secretions. MSC EV may have potential for use as therapeutic agents or vectors, including a clinical application of MSC EV for treatment of severe graft-versus-host disease (Kordelas, L. et al., 2014, Leukemia, 28(4):970-973). However, there is insufficient knowledge about the molecular, as well as biochemical and genomic, mechanisms by which MSC EV exert putative immune modulation, in contrast to tumor or tumor stroma derived EV, which have been reported to possess a number of immunosuppressive protein ligands (e.g., PD-L1, PD-L2, PD-1, FasL, TGF-β1, CD39, CD73, Galectin-1, CTL4). Certain ligands (PD-L1, TGF-β1, and Galectin) can be transferred by murine MSC derived EV to lymphocytes, inducing their autocrine production of IL-10 and TGF-β1. Similarly, immune modulatory proteins typically associated with paracrine signaling in MSC are also carried by MSC EV (e.g., DO, NO, PGE2, TGF-β1, adenosine, IL-10), although their functional relevance in association with EV is unclear.

In experiments similar to those described in Example 2, this Example describes experiments in which extracellular vesicles (EV) isolated from canine Wharton's Jelly derived MSC (WJ-MSC) were assayed for their ability to suppress peripheral blood CD4+ T lymphocyte proliferation through a transforming growth factor-β (TGF-β) signaling mechanism. The experiments were further designed to assess whether WJ-MSC EV can suppress CD4⁺ T helper cells within PBMC in a manner that is consistent with the effects of the parent WJ-MSC.

Materials and Methods

Wharton's Jelly MSC

Animals, tissue collections, and WJ-MSC isolation: The study described in this Example received prior approval by the Institutional Animal and Care Usage Committee of Tufts University. Privately owned healthy donor dams from various breeds, e.g., Corgi, American Staffordshire Terrier, Labrador Retriever, Golden Retriever, Rottweiler and German Shepherd, between 1-10 years old participated under owner consent at the time of elective Cesarean section. Donors (adult females) were tested negative for Brucella canis, Dirofilaria immitis, Ehrlichia canis, Borrelia burgdorferi, and Anaplasma phagocytophilum antigens prior to breeding. All puppies removed by Cesarean section received standard of care and were returned to their owners. Fresh placental tissue was collected under aseptic conditions and were processed within 24 hours. Wharton's Jelly (WJ) tissue was dissected away from the umbilical artery and vein, placed in cold phosphate buffered saline (PBS), and minced with a scalpel. Explanted tissue fragments were washed three times with PBS through a 100 μm filter, and incubated in 3 mg/ml collagenase/dispase (Sigma-Aldrich, St Louis Mo.) at 37° C. for one hour using a procedure modified from Lee, K. S et al., 2013, Res Vet Sci, 94(1):144-151). The tissue digest was filtered through a 100 μm filter, and cells were plated at low density (passage 0, approximately 2×10³ cells/cm³) in Alpha-MEM (Sigma-Aldrich, St Louis, Mo.), supplemented with 15% fetal bovine serum (Hyclone, GE Life Sciences, Little Chalfont, UK), 10,000 U/ml penicillin-streptomycin, and 2 mM L-glutamine (Life Technologies, Carlsbad, Calif.), called ‘cAlpha-MEM’. Cells adhered to culture plates for 48 hours prior to changing the medium every 48-72 hours thereafter. Cells were routinely passaged using 0.25% trypsin with EDTA (HyClone), washed, and cryopreserved (−160° C.) at passage 1 in 60% FBS, 30% cAlpha-MEM, and 10% DMSO (10%) until further use.

Flow cytometry: Cells were incubated with primary antibodies in 5% FBS for 30 minutes on ice, including anti-CD34-PE (AbD Serotec, mouse anti-dog clone 1H6), CD44-APC (AbD Serotec, rat anti-dog clone YKIX337.8.7), CD45-APC (AbD Serotec, rat anti-dog clone YKIX716.13), MHCII-FITC (AbD Serotec, rat anti-dog clone YKIX334.2), CD90-APC (eBioscience, rat anti-dog clone YKIX337.217). A viability marker (7AAD) was applied to all samples for gating of viable cells. After gating on the viable cells, cell phenotype was determined by comparing histograms of the stained samples to the isotype control. Samples were evaluated using an Accuri C4 (Accuri Cytometers Inc), with a minimum of 100,000 events analyzed using CFlow Plus v. 1.0.208.2.

Trilineage differentiation: All cells were differentiated at passage 3 and were plated in 6 well plates using 1.4×10⁵ cells per well in aMEM containing 1× L-glutamine, 100 U/mL penicillin/streptomycin, and 15% FBS. Cells were changed to differentiation or control medium upon reaching 80% confluence. DMEM low glucose with 2 mM L-glutamine, 100 U/mL penicillin/streptomycin, and 5-10% FBS was used as control medium for all three lineages. To induce adipogenesis, cells were incubated for 12 days in DMEM (Gibco, 31053) containing 10% rabbit serum (Sigma, R4505), 1 μM dexamethasone (Sigma, D2915), 10 μM insulin (Humulin N U-100, Lilly), and 200 μM indomethacin (Sigma, I7378). Adipogenesis was assessed by Oil Red O staining.

To induce osteogenesis, cells were incubated for 21 days in DMEM (Gibco, 31053) containing 10% FBS (Hyclone, sh3007003), 100 nM dexamethasone (Sigma, D2915), 10 mM b-glycerophosphate (Sigma, G5422), and 50 μM L-ascorbic-acid-2-phosphate (Sigma, A8960), and 2 mM L-glutamine. Osteogenesis was assessed using the StemPro Osteogenic Kit staining protocol using Alizarin Red. To induce chondrogenesis, cells were incubated for 21 days in DMEM (Gibco, 31053) containing 1 mM sodium pyruvate (Gibco, 11360-070), 100 nM dexamethasone (Sigma, D2915), 50 μM L-ascorbic-acid-2-phosphate (Sigma, A8960), 40 μg/mL L-proline (Sigma, P5607), 1% ITS (Lonza, 17-838Z), 50 ng/mL BMP-2 (Millipore, GF166), and 50 ng/mL TGFb1 (Cell Signaling, 8915LC). Chondrogenesis was assessed using Alcian Blue staining.

Quantitative real-time PCR: Total RNA was isolated from WJ-MSC using the RNAeasy kit (Qiagen, Hilden, Germany) as per the manufacturer's instructions. RNA concentrations and quality were determined with the RNA 6000 Nano Assay Kit and the Bioanalyzer 2100 (Agilent Technologies, Santa Clara, Calif.). All RNA samples had RNA integrity numbers>8. Complimentary DNA was generated by using the RT² First Strand Synthesis kit (Qiagen), and heat cycling at 42° C. for 15 minutes followed by 95° C. for 5 minutes prior to placing on ice. 5 μl of cDNA was mixed with RT2 SYBER Green Mastermix, RNase free water, and 10 μM primer. mRNA expression of CD73 (Qiagen, PPF01104A), CD44 (Qiagen, PPF00491A), MCHII (Qiagen, PPF01028A), CD45 (Qiagen, PPF10210A), CD34 (Qiagen, PPF00586A), and CD90 and CD105 (Invitrogen) was measured. HPRT and RP519 were used as housekeeping genes for normalization of Ct data.

WJ-MSC Extracellular Vesicles: Isolation and Characterization

Serum-free culture and stepwise ultracentrifugation: WJ-MSC were thawed and seeded at low density (˜6000 cells/cm²) in cAlpha-MEM. Once 70% confluent, cells were transitioned to serum-free defined chemical medium (‘DCM’) modified from Lai et al. (2011, Regen Med., 6(4):481-492), which contained DMEM (Life Technologies) supplemented with 25 μM HEPES (Life Technologies), 1× penicillin-streptomycin and L-glutamine (Life Technologies), Insulin-Transferrin-Selenium premix (Gibco), 5 ng/ml recombinant human fibroblast growth factor 2 (Invitrogen, Carlsbad, Calif.), and 5 ng/ml recombinant human platelet-derived growth factor AB (also Invitrogen). Cells were transferred from serum containing medium to 50% DCM plus 50% cAlpha-MEM for 24 hours, washed with PBS, and then the medium was replaced with 100% DCM for 48 hours. Conditioned medium was collected after 48 hours. Supernatant was collected after each of the following steps in centrifugation: 300×g for 10 minutes, 2,000×g for 10 minutes, and 10,000×g for 30 minutes (Eppendorf 5810). The remaining supernatant was then diluted 1:1 with PBS and ultracentrifuged at 100,000×g for 70 minutes. (Beckman Coulter Optima™ L-90K Ultracentrifuge, Brea, Calif.) using a 45Ti rotor (k-factor 133). The pellet was then resuspended in 1 ml PBS for downstream applications.

Particle size distribution using nanoparticle tracking analysis (NTA): Samples were analyzed using a NanoSight N300 unit (Malvern) equipped with a 488 nm (blue) laser module and Nanoparticle Tracking Analysis 3.0 software. All samples were diluted in sterile PBS to a concentration of 1-10×10⁸ particles/mL for analysis. Specific NTA settings were optimized for each sample, with fixed settings of temperature (23° C.), screen gain (1.0), infusion flow rate (5 μL/min), and camera level set at 12-14 depending on sample characteristics. Five videos were recorded for each sample (30-120 s video length) with all settings remaining constant within each sample source to minimize variation. The detection threshold was set to 5 using auto blur and auto max jump distance settings, with a minimum analysis of 200 valid tracks per video and a minimum of 1000 valid tracks per sample. The NTA unit was periodically evaluated for accuracy of size determination using polystyrene beads of known size (100 and 200 nm).

Density gradient separation of WJ-MSC EV samples—buoyancy measurements based on TSG101 expression: Gradients were constructed with iodixanol (OptiPrep™ Density Gradient Medium, 60% aqueous preparation, Sigma) diluted in gradient buffer containing 0.25 M sucrose, 10 mM Tris and 1 mM EDTA, at pH 7.4. A concentrated EV sample in a volume of 500 μl PBS was supplemented with 0.25 M sucrose and 1 mM EDTA and was mixed with 1 ml of 60% iodixanol to give 1.5 ml of sample in 40% iodixanol. The sample was loaded in the bottom of Ultra-Clear™ ½ by 2 inch centrifuge tubes (Beckman Coulter). Iodixanol solutions were layered on top as follows: 1.2 ml of 30%, 1.2 ml of 20%, 1.4 ml of 10%. A control gradient prepared in the same manner, minus the EV sample, was performed simultaneously. Tubes were placed in an SW55Ti rotor and subjected to 2 hours of 350,000×g at 4° C. in an Optima™ L-90K ultracentrifuge (Beckman Coulter). Following centrifugation, 8 fractions of 625 μl were removed from the top of the tube, leaving a small amount of residual volume or 9^{th} fraction). Fraction density was measured by adding 20 μl of each fraction with 80 μl water into duplicate wells of 96 well plate and measuring absorbance at 340 nm in a plate reader compared to a linear standard curve of 0, 10, 20, 30, 40 and 60% iodixanol, also diluted 1:4 in water. Expected density of iodixanol in 0.25 M sucrose buffer was taken from the Axis-Shield OptiPrep application sheet from the manufacturer, and density of the collected fractions was calculated from the standard curve. NTA was performed on collected fractions. Fractions were then concentrated to a volume of 175 μl with ULTRA®-10K regenerated cellulose 10,000 MWCO centrifugal filters (Amicon Ultra 0.5 ml). The BCA assay was performed on the concentrated fractions for protein quantification, NTA for particle count, and immunoblot for TSG101 (Tumor Susceptibility Gene 101) as described for Western Blots.

Transmission electron microscopy (TEM): EV were diluted in PBS and adhered to copper mesh SPI 200 SuperGrids™ (2620C, West Chester, Pa.). Uranyl acetate (1%) in deionized water was used for negative staining. Images were obtained at 3870× and 4135× magnification using an FEI Tecnai™ Spirit 12 electron microscope.

Peripheral Blood Mononuclear Cell (PBMC) Suppression Assays

PBMC responder assay: Twelve healthy adult purpose-bread Beagle dogs (5 neutered males, 7 spayed females) housed at the Laboratory Animal Medicine department at the Cummings School of Veterinary Medicine at Tufts University served as blood donors under an approved protocol. WJ-MSC or WJ-MSC were tested in triplicate against a minimum of 3 different PBMC donor cell samples (see Figure legends for ‘n’ of each experiment). Healthy status was confirmed by clinical examination, and hematological and serum biochemical testing within six months of blood sampling. All dogs were fasted for 12 hours prior to peripheral blood sampling. Peripheral blood samples were taken via jugular venipuncture using a 21 gauge needle, and blood was immediately placed into an EDTA collection tube and rotated 1-2 times gently. Blood was diluted 1:1 with cold PBS or centrifuged (1300×g, 10 min) prior to dilution 1:2 in PBS, followed by density-gradient centrifugation using Ficoll-Paque (density 1.077; GE Healthcare Life Sciences) to harvest peripheral blood mononuclear cells (PBMC). PBMC were washed in RPMI (Life Technologies) supplemented with 10% heat-inactivated fetal bovine serum (Gibco), 25 μM HEPES (Life Technologies), 100 μM β-mercaptoethanol (Sigma-Aldrich), 10,000 U/ml penicillin-streptomycin, 2 mM L-glutamine (Life Technologies), and 2.2 mg/mL of sodium bicarbonate (Thermo Fisher Scientific) (complete RPMI, ‘cRPMI’). For mitogen induced lymphocytes proliferation assays, PBMC were suspended in 1 ml cRPMI and 0.5 μof 10 mM carboxyfluorescein succinimidyl ester (CFSE, Thermo Fisher Scientific) and were incubated (10 min). For initial characterization of WJ-MSC effects on responder PBMC, PBMC were plated on a 24-well plate on the top of a 0.4 μm Transwell (Corning) across from the WJ-MSC in a 10:1 ratio (PBMC:MSC) in a total volume of 1 ml medium (n=3 technical replicates/sample). All plates were incubated at 37° C. in the dark for 72 hours prior to analysis.

Dose-responses of TV MSC-EV: To investigate the dose response of PBMC to WJ-MSC EV, the WJ-MSC were plated on a 96-well plate in cRPMI with addition of 1 mM ATP, with or without 5 μg/ml Concanavalin A (ConA, Sigma-Aldrich). Responder PBMC were incubated with WJ-MSC EV in a total volume of 200 μL medium (n=3 technical replicates per sample). PBMC were incubated with WJ-MSC EV in a 1:10², 1:10³, or 1:10⁴ (PBMC:EV) ratio. After 72 hours of incubation, the PBMC were collected and surface stained for CD4 (rat anti-canine CD4-Alexa 647, clone YKIX302.9, AbD Serotec). Cells were stained for 30 minutes, washed twice with cold PBS and 5% FBS, and resuspended in 200 μl PBS and 5% FBS with 5 μl 7AAD (Becton Dickinson) for analysis by flow cytometry (Accuri C6, Becton Dickinson). The effect of WJ-MSC or WJ-MSC EV on CD4-positive (CD4⁺) T cell (i.e., T helper cell) proliferation (percentage of cells proliferating, number of divisions for proliferating cells) was measured by evaluating CFSE fluorescence utilizing the FlowJo® proliferation platform (FlowJo, V10, Ashland, Oreg.) after gating on the viable CD4⁺ lymphocyte population.

EV depletion and enzymatic digestion experiments: EV were isolated as described, resuspended in 5 ml PBS, and divided into 5 fractions of 1 ml each. The fractions were either left untreated, or were treated with 0.1% Triton-X, 2 μg/ml RNAse A, or both RNAse A and proteinase K for 30 minutes at 37° C. The samples were then diluted with PBS and centrifuged at 100,000×g for 70 minutes. The EV sediment was resuspended in PBS and particle numbers were quantified using nanoparticle tracking analysis (NTA) as described supra. A ratio of 1 PBMC to 10⁴ EV was cultured in 200 μl of cRPMI with 1 mM ATP, with or without 5 μg/ml ConA for 72 hours (n=3 technical replicates per sample). WJ-MSC EV depleted controls: following exosome isolation, the sediment was resuspended in 1 ml PBS and filtered through a 10 kDa (Amicon Ultracel, Sigma) or 50 kDa (Sartorius Vivaspin 4, Gottingen, Germany) molecular weight cutoff filter (MWCO) and centrifuged (4,000×g) for 15 minutes. The filtrate was collected. The retentate was resuspended in 1 ml PBS, and particle content was evaluated by NTA. A volume of EV from retentate to generate a ratio of 10⁴ EV:1 PBMC, or an equivalent volume of filtrate, were cocultured with PBMC as described. In addition, the supernatant generated by stepwise ultracentrifugation (100,000×g) was employed as an EV depleted sample. Chemical inhibition of EV biogenesis was achieved using GW4869 (6 μM), an inhibitor of neutral sphingomyelinase (Guo, Bellingham et al. 2015) for 48 hours, then re-plating GW4869-treated or non-treated WJ-MSC across 0.4 μm Transwell from PBMC in a 1:10 ratio for 72 hours prior to collection of PBMC and evaluation of lymphocyte suppression. As well, WJ-MSC EV, EV (10⁴:1 PBMC) from conditioned medium of non-MSC fibroblasts (canine left ventricular cardiac fibroblasts) were evaluated for suppression of PBMC.

Functional evaluation of WJ-MSC EV TGF-β: Ten μM of SB4312 (Tocris, Bristol, UK), a specific TGF-βRI inhibitor (Hasan, Neumann et al. 2015), 50 μM of ZM241385 (Tocris), a specific adenosine2A receptor inhibitor, or 0.1 μg/ml of TGF-β 1,2,3 neutralizing antibody (R&D Systems, Minneapolis, Minn.) was added to PBMC plus WJ-MSC EV or PBMC in coculture with WJ-MSC at the beginning of the 72-hour incubation period. Alternatively, 5, 10, or 50 ng/ml of recombinant human TGF-β1 (R&D Systems) or TGF-β3 (Sigma) were added to ConA stimulated PBMC cultures. For disruption of the heparin sulfate side chains on TGFβRIII (beta-glycan), WJ-MSC EV were incubated with heparinase III (Sigma) or heat-inactivated heparinase (control) at 0.006 U/ml for 3 hours at 37° C. according to the method of Webber et al. (2015, Oncogene, 34(3):290-302). The WJ-MSC EV were washed by resuspending in PBS and ultracentrifugation at 100,000×g for 70 minutes prior to application in PBMC coculture.

Bead-assisted flow cytometry of TGF-β on WJ-MSC EV. A total volume of 1×10¹⁰ EV was incubated with 10 μl (1.2×10⁷) 3.9 μm latex beads for 15 minutes at room temperature. The sample was diluted to a volume of 1 ml with PBS, and the sample was incubated overnight on a tube rotator at room temperature. The bead-EV sample was pelleted by centrifugation for 3 minutes at 1500×g. The supernatant was removed, and 1% BSA was added to a total volume of 500 μl for 30 minutes. The sample was pelleted again, the supernatant removed, and the sample resuspended in a final volume of 100 μl 0.1% BSA. Samples were incubated for 30 minutes with 1 μg of mouse anti-TGF-β1, 2, 3 (R&D Systems), isotype, or secondary antibody alone; 10 μl of sample was used incubation with each antibody. Samples were washed, then incubated with secondary antibody for 30 minutes prior to washing and evaluating by flow cytometry.

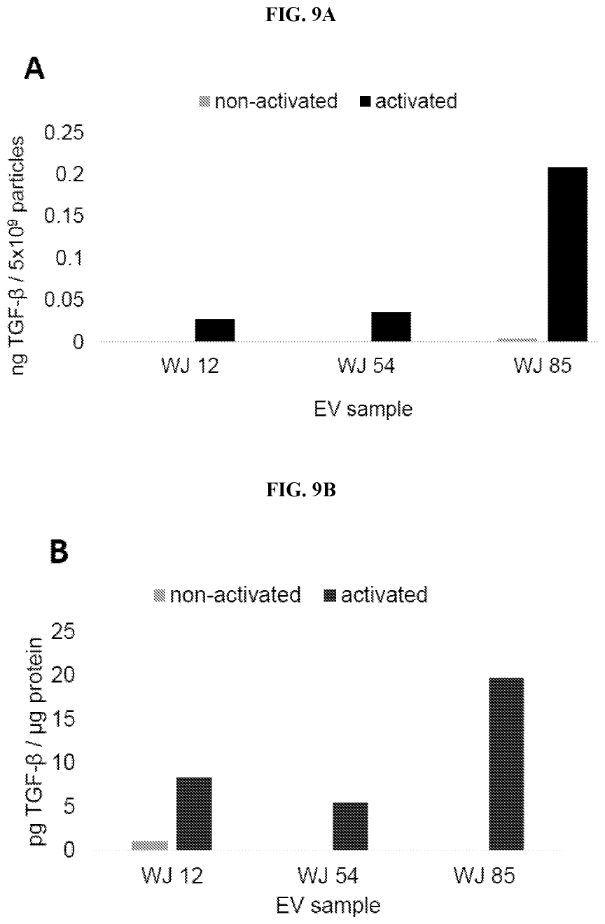

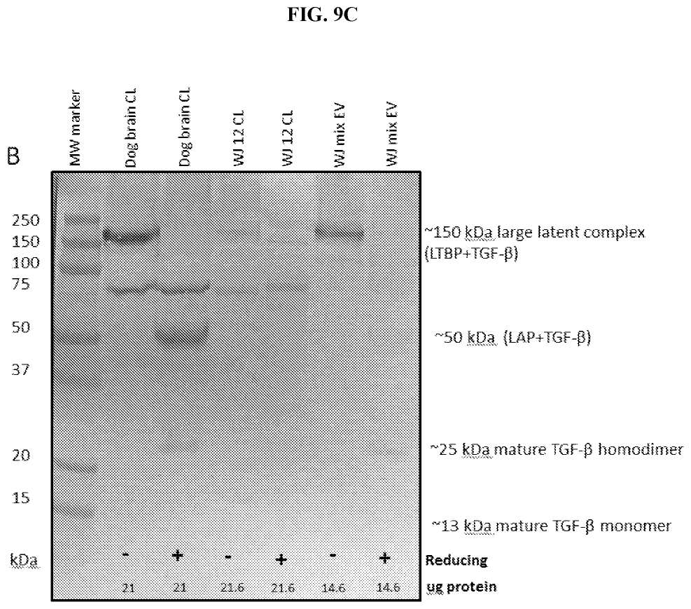

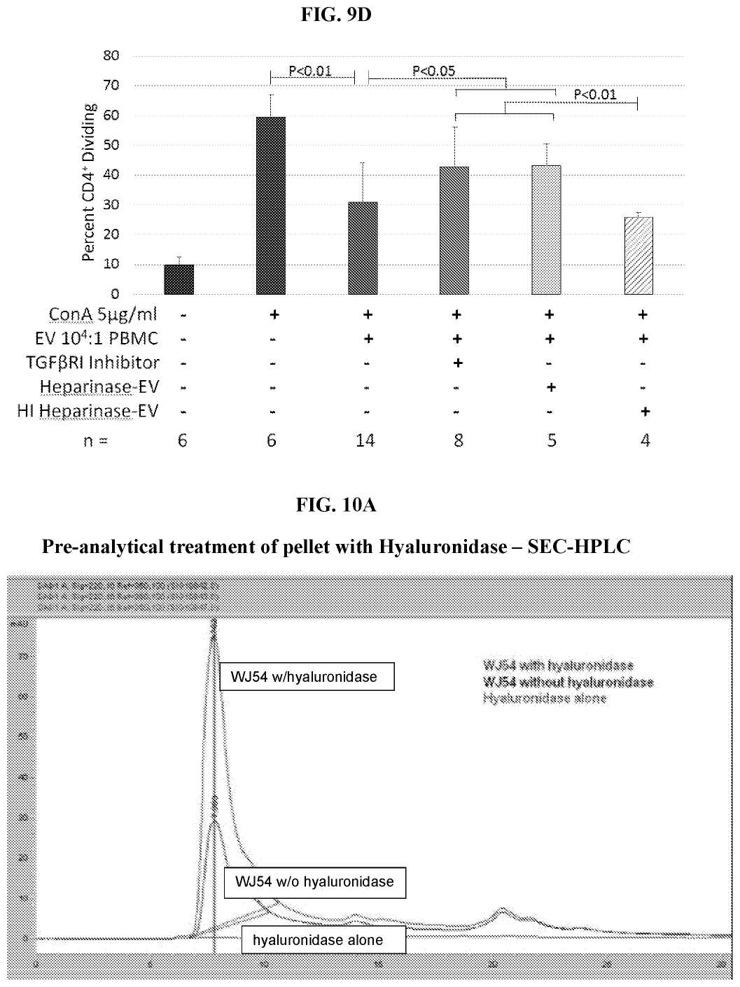

Enzyme linked immunosorbent assay (ELISA) to evaluate content of latent versus mature TGF-β on WJ-MSC EV: ELISA immunoassay was performed using the TGF-β1 Quantikine ELISA kit (R & D Systems) on WJ-MSC EV sediments, as per manufacturer's instructions, with exception that for some samples, pretreatment with acid was omitted (in order to measure the quantity of native active TGF-β form only). Samples were diluted 1:4 for ELISA analysis prior to acid activation where appropriate and absorbance was measured at 450 nm. (e.g., FIGS. 9A and 9B).

Western Blots: Protein was solubilized from PBMC, CD4⁺ T cells, or WJ-MSC using m-PER (Thermo Scientific). Protein obtained from cell extracts or EV was quantified by a bicinchoninic acid kit (Pierce BCA Protein Kit, Thermo Scientific). Western blotting was performed using the iBLOT™ kit (Becton Dickinson) according to manufacturer's instructions. Equal amounts of protein were loaded into each lane of Bolt™ 4-12% Bis-Tris gels (Invitrogen), resolved in Bolt™ MES SDS running buffer (Invitrogen), and electroblotted onto nitrocellulose membranes. The iBIND™ Flex kit was used for blocking and antibody application. Antibodies used included anti-TGFβRI antibody (Abcam, clone ab125310) at 1:500, anti-TGFβ-1 antibody (Abcam, ab190503) at 1:500 dilution, anti-TSG101 (BD Biosciences 612696) at 1:1000 dilution, anti-PDC6I (Alix) (Abcam ab76608) at 1:1000 dilution, and anti-calnexin (Abcam ab75801) at 1:1000 dilution. Biotinylated conjugated horse anti-mouse antibody (BA-2000, Vector Laboratories) or biotinylated goat anti-rabbit (BA-1000) at 1:40 dilution was used as a secondary antibody, and detection was performed using the Vectastain ABC Kit (Vector Laboratories), followed by use of the Peroxidase DAB substrate kit (Vector Laboratories). Control cells included HeLa cells, Mardin-Darby Canine Kidney (MDCK) cells and EV, and dog brain cells isolated from donated tissue after client approval from euthanized animals.

Statistical Analysis: The distributions of percent dividing CD4⁺ cells were explored for normality through descriptive statistics, and pairwise statistical comparisons were performed using a paired-sample t-test. Comparisons between 3 or more groups were made using ANOVA, followed by Tukey multiple means comparison post-test. Analyses were performed using SPSS (Version 24, IBM). Pearson's correlation coefficients were performed in Microsoft Excel (Version 15.24). For all analyses, statistical significance was set at p<0.05. Values are expressed as mean ±standard deviation.

The findings and results of the experiments described in this Example are further described hereinbelow.

### Wharton's Jelly-MSC (WJ-MSC) Exhibited MSC Phenotype and Differentiation Capacity

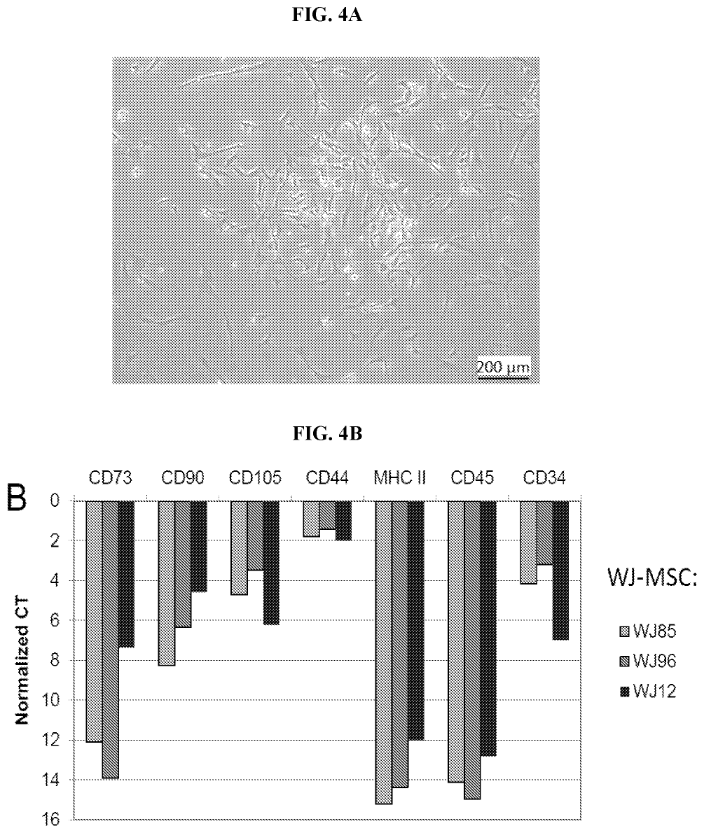

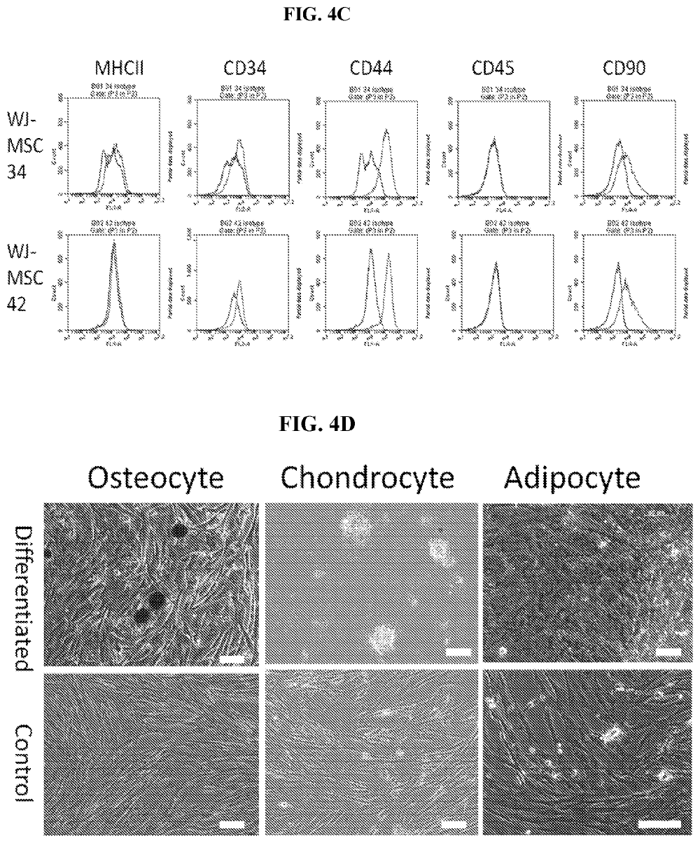

The WJ-MSC isolated from canine Wharton's Jelly exhibited plastic adherence colony formation, surface phenotype, and trilineage differentiation (FIGS. 4A-4D). Specifically, WJ-MSC retained a typical MSC-like (spindle shaped, elongated, fibroblastic) morphology at least through passage 6 (FIG. 4A). WJ-MSC also expressed genes encoding phenotypic markers of MSC (CD44, CD73, CD90, CD105, not CD45 or MHCII) (FIG. 4B), which were largely corroborated by flow cytometry (CD44^{pos}, CD90^{pos}, CD105^{pos}, CD45^{neg}, MHCII^{neg}) (FIG. 4C). Discordance was observed for CD73 and CD34 whose expression was evident on qPCR, but absent on flow cytometry. WJ-MSC showed the capacity for differentiation to osteocytes, chondrocytes, and adipocytes (FIG. 4D).

WJ-MSC EV Size Distribution and Morphology was Consistent with ‘Small EV’

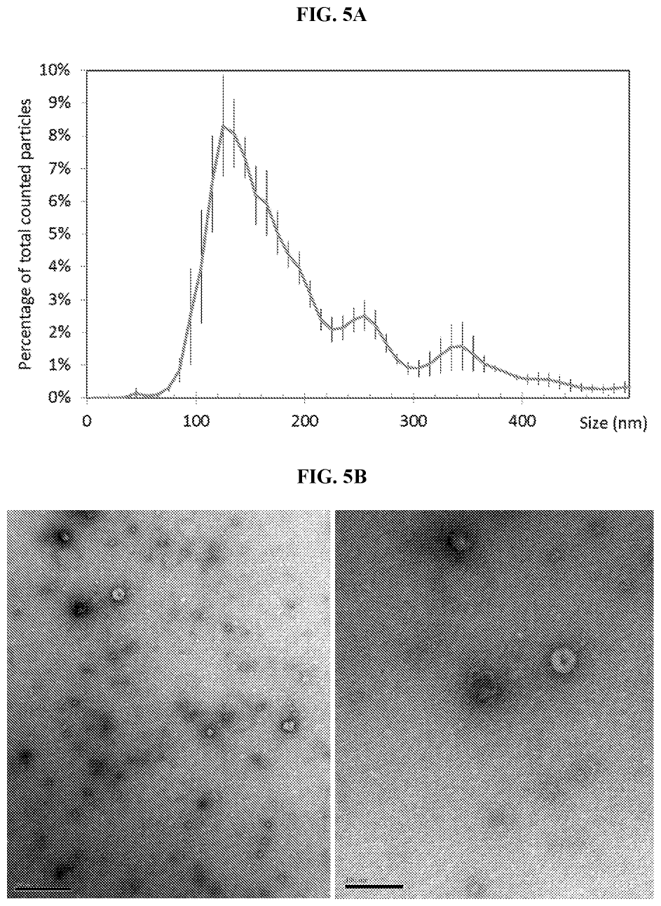

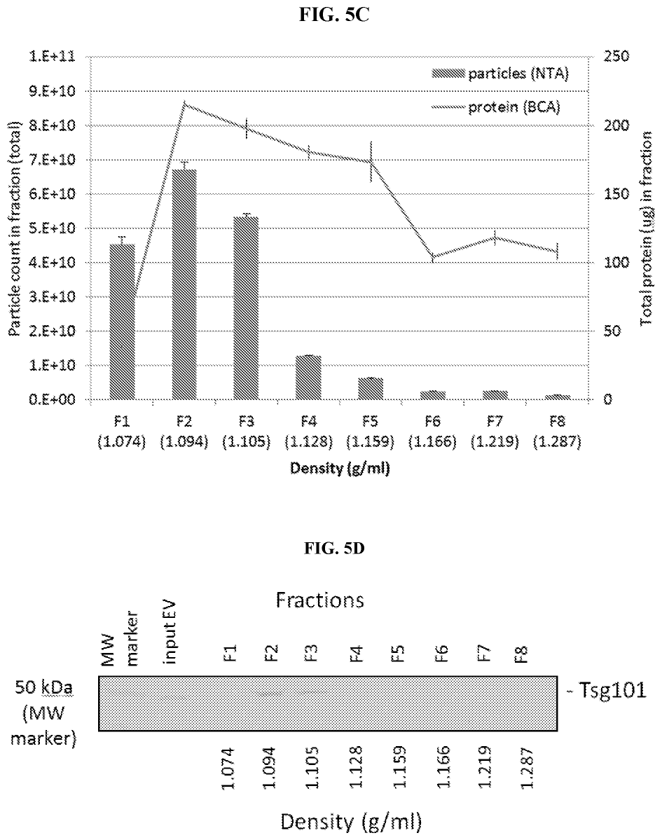

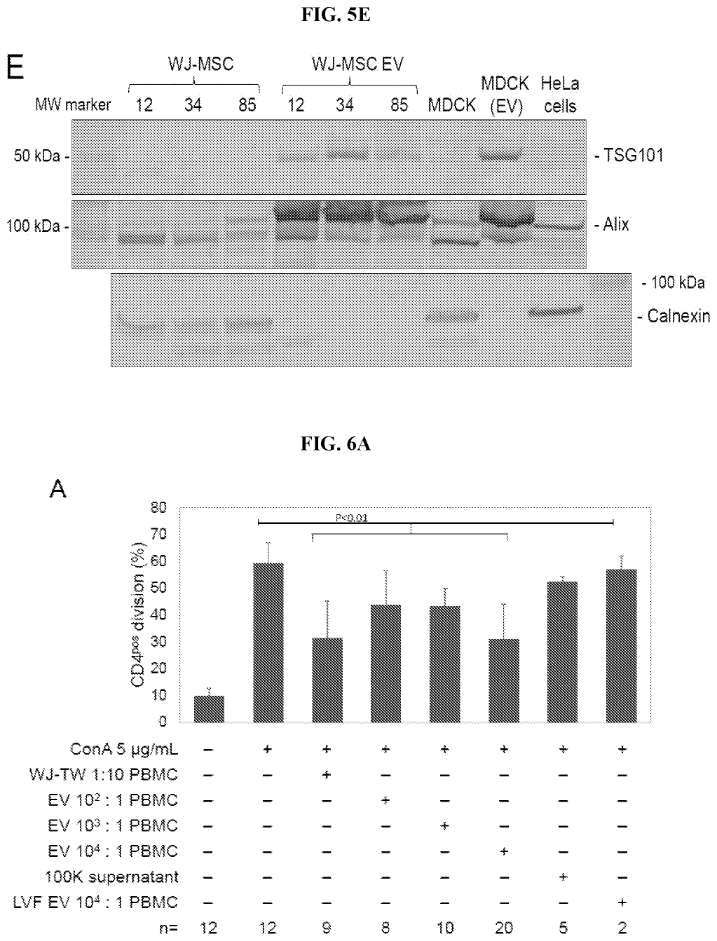

The mode and mean particle size derived from the composite data of 5 WJ-MSC EV lines was 125 nm and 199 nm, respectively, with 76% of all EV ranging from 50-250 nm, consistent with the size characteristics of small EV (FIG. 5A). The ultrastructural morphology of WJ-MSC EV (cup shaped) by TEM was also consistent with small EV (range ˜50 -100 nm), but included many smaller spherical or donut-shaped structures (˜20 nm) adding to the diversity of vesicles produced by a single cell type (FIG. 5B). WJ-MSC EV, initially isolated from cell culture supernatant by stepwise ultracentrifugation and applied to a density gradient (40, 30, and 10% Optiprep), were observed to be concentrated in fractions 1, 2, and 3 out of 9 fractions based on particle distribution (NTA) and protein content (BCA) (FIG. 5C), and fractions 2 and 3 based on TSG101 content. Fractions 2 and 3 correspond to a buoyant density of 1.094-1.105 g/ml (FIG. 5D). On western blots, WJ-MSC EV were enriched for Tsg101 and Alix, proteins associated with exosome biogenesis, relative to parent WJ-MSC (FIG. 5E). Calnexin, an endoplasmic reticulum protein, was detected in parent WJ-MSC, but not in the respective WJ-MSC EV, demonstrating EV specificity of the isolates.

### WJ-MSC or WJ-MSC EV Suppress CD4⁺ T Cell Proliferation

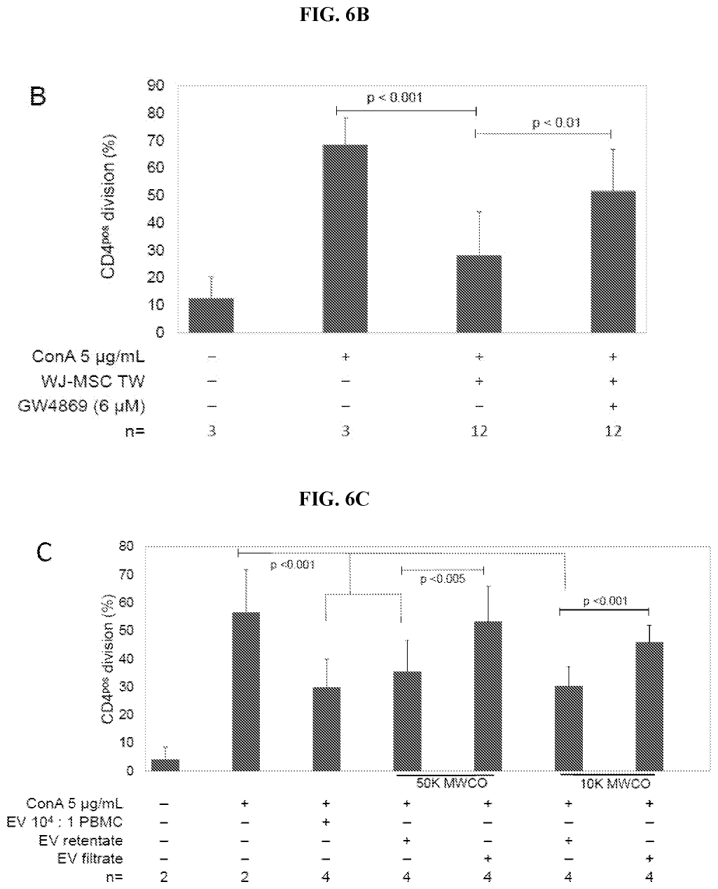

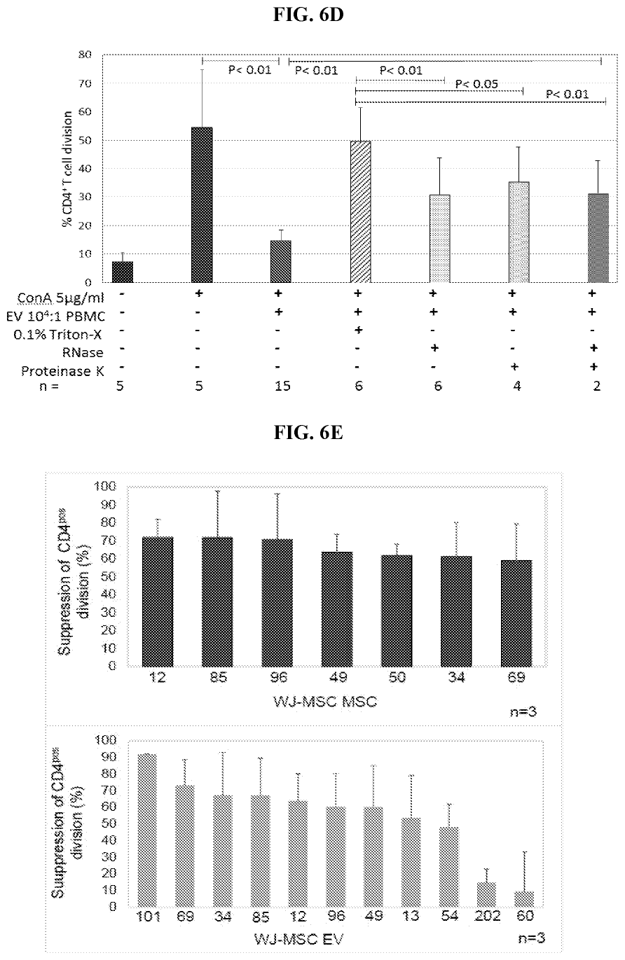

The dose-response of WJ-MSC EV immunomodulatory capacity was evaluated through coculture of EV with ConA-stimulated PBMC. This showed that WJ-MSC EV mediated suppression of ConA stimulated CD4^{pos} T cell proliferation was dose dependent (FIG. 6A). Dilutions of 10² and 10³ EV:PBMC resulted in significantly reduced suppression relative to PBMC with ConA alone (43.7%±12.8% and 42.9%±8.2% vs 59.2%±8.7%, respectively, p<0.01), whereas 10⁴ EV:PBMC suppressed T cell proliferation further (30.8%±13.2%), to an extent that was indistinguishable from WJ-MSC at 1 MSC to 10 PBMC (32.7%±15%) (FIG. 6A). Both WJ-MSC (across transwell) or WJ-MSC EV suppressed the percentage of CD4⁺ T cells that proliferated in response to mitogen, but not the number of cell divisions for dividing cells (not shown). Hence, the percentage of CD4⁺ T cells that proliferated in response to mitogen was utilized as the endpoint in PBMC responder assays going forward. Supernatant from EV sedimentation (equivalent v/v) did not suppress CD4⁺ T cell division, suggesting that the effect derived specifically from the EV-enriched sediment fraction. Similarly, there was no effect of EV from non-MSC cardiac fibroblasts on T cell proliferation (FIG. 6A), suggesting that the effect was unique to MSC derived EV. Following these findings, a concentration of 10⁴ WJ-MSC EV per PBMC was utilized for subsequent experiments.

### Depletion of EV Ameliorated Lymphocyte Suppression

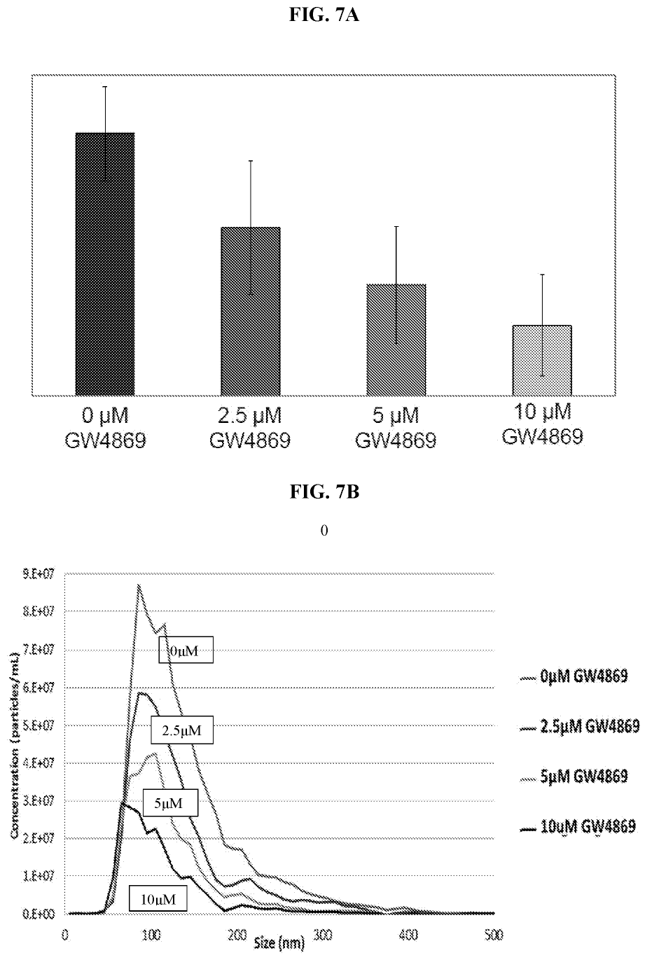

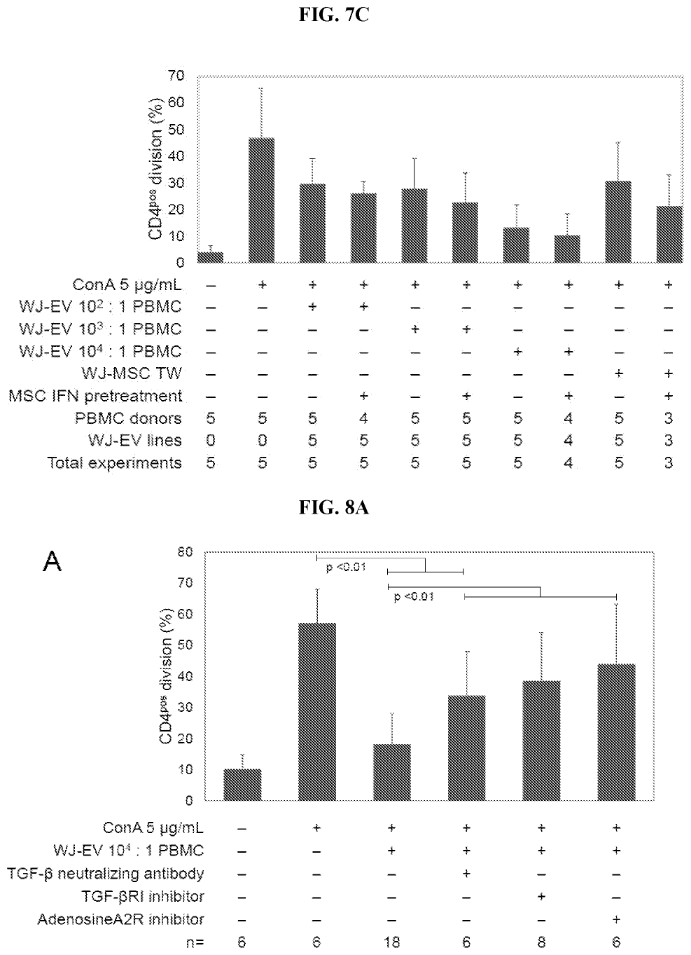

The neutral sphingomyelinase (NSMase) inhibitor, GW4869, was employed to assess exosome output from MSC. Increasing doses GW4869 were applied to the WJ-MSC to assess exosome output that resulted in maximal suppression without an unacceptable loss of viability at 5-10 μM (FIGS. 7A and 7B). Accordingly, WJ-MSC were pre-treated for 48 hours with GW4859 at 6 μM prior to culture in fresh medium across a Transwell from PBMC. Pretreatment with GW4859 to inhibit EV release suppressed the anti-proliferative effect of WJ-MSC on T cell division in the presence of Con A (51.7±13.5%, relative to 28.2±1.4%) in treated and untreated WJ-MSC. (p<0.01) (FIG. 6B). WJ-MSC EV derived by stepwise ultracentrifugation and resuspended in PBS were filtered through 10 or 50 kDa MWCO filters in order to further purify WJ-MSC EV and to ensure that non-EV particles were not responsible for suppression of CD4⁺ T cells. The filtrate and retentate were then compared for their ability to suppress CD4⁺ T cell division. The filtrates of both 10 kDa and 50 kDa MWCO filtered samples were inactive; however, the retentates of both the 10 kDa and 50 kDa MWCO filtered samples reduced CD4⁺ division (FIG. 6C), consistent with the major effect on proliferation caused by the WJ-MSC EV themselves rather than by a soluble component co-sedimented during EV preparation. Triton-X, which obliterates cell or EV membranes, completely abrogated the suppressive effects of EV (FIG. 6D, p<0.01), consistent with the active role of WJ-MSC EV. To a lesser extent, exposure to RNase and proteinase K also reduced the WJ-MSC EV induced suppression of cell division (FIG. 6D, p<0.01).

### Reproducibility of WJ-MSC and WJ-MSC EV Activity

Across all cells lines, WJ-MSC and WJ-MSC EV consistently suppressed ConA stimulated T cell proliferation (FIG. 6E). Activity of WJ-MSC EV to suppress CD4⁺ T cell division was also consistent within WJ-MSC donor cell lines (FIGS. 7A and 7B). Estimated from NTA, the number of EV released by each WJ-MSC varied among cell lines. On average, WJ-MSC generated 5780±3291 EV per cell, as shown below in Table 1. In Table 1, variation in number of EV released per cell may be observed. The average number of EV released per cell was 5780±3291. Variation exists between and within cell lines.

| WJ Line | Number of Cells | Number of EV/mL | EV/Cell |
|---|---|---|---|
| 12 | 2.20E+07 | 2.20E+011 | 10000.0 |
| 13 | 1.07E+07 | 6.20E+10 | 5794.4 |
| 34 | 3.00E+07 | 4.12E+11 | 13733.3 |
| 34 | 6.00E+06 | 5.10E+10 | 8500.0 |
| 49 | 1.70E+07 | 1.49E+11 | 8764.7 |
| 49 | 4.40E+07 | 2.00E+11 | 4545.5 |
| 49 | 6.28E+07 | 1.95E+11 | 3105.1 |
| 52 | 5.80E+06 | 2.40E+10 | 4137.9 |
| 69 | 2.40E+07 | 9.20E+10 | 3833.3 |
| 76 | 9.00E+06 | 5.90E+10 | 6555.6 |
| 85 | 6.70E+07 | 2.80E+11 | 4179.1 |
| 85 | 6.60E+07 | 5.80E+10 | 878.8 |
| 85 | 1.07E+09 | 3.60E+11 | 336.4 |
| 85 | 8.00E+07 | 3.90E+11 | 4875.0 |
| 96 | 3.00E+07 | 1.80E+11 | 6000.0 |
| 96 | 5.00E+07 | 3.50E+11 | 7000.0 |
| 111 | 1.80E+07 | 1.51E+11 | 8388.9 |
| 157 | 4.40E+07 | 1.50E+11 | 3409.1 |

### IFN-γ Pre-Conditioning of WJ-MSC Did Not Increase Suppression of T Cell Division by WJ-MSC or WJ-MSC EV

Pretreatment of WJ-MSC with 500 ng IFN-γ for 48 hours prior to collection of WJ-MSC EV was performed to determine if such pretreatment would augment either WJ-MSC (across transwell) or WJ-MSC EV activity. While there were trends in further suppression by WJ-MSC or WJ-MSC EV following pre-conditioning of WJ-MSC, these effects were not statistically significant. (FIG. 7C).

### TGF-β and Adenosine Signaling are Mechanisms of WJ-MSC EV Mediated Suppression of CD4⁺ T Cells

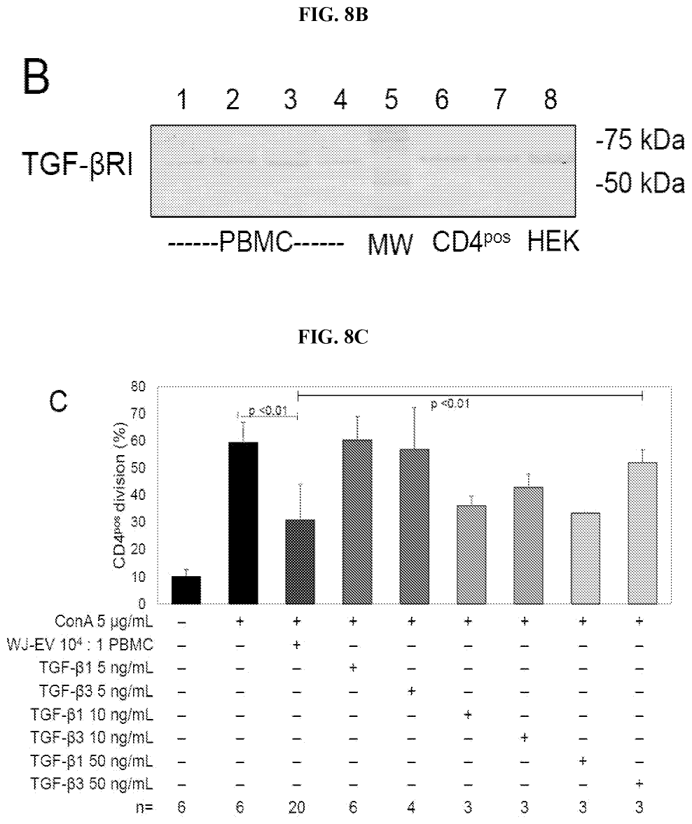

To determine if TGF-β1 or adenosine contribute to EV induced immune modulation, WJ-MSC EV and PBMC were cocultured with TGF-β (1, 2, and 3) neutralizing antibody or with pharmacological inhibitors of TGFβRI, adenosine 2A receptors, or both. Neutralization of TGF-β (1, 2, and 3) with functional antibody significantly reduced suppression of CD4+ (CD4^{pos}) cell division (FIG. 8A, p<0.001). Similarly, blockade of TGFβRI or the adenosine 2A receptor significantly reduced the effect of WJ-MSC EV to suppress T cell division (FIG. 8A, p<0.01).

### Exogenous Soluble TGF-β Suppresses EV

The presence of TGFβRI on both PBMC and isolated CD4⁺ T cells was demonstrated by Western blot (FIG. 8B). This suggested that a direct interaction of TGF-β with this receptor on CD4⁺ T cells could account for anti-proliferative effects. In support of this, addition of 10 ng/ml, but not 5 ng/ml, of TGF-β1 or TGF-β3 suppressed mitogen driven T cell division (FIG. 8C).

In this Example, the data and results demonstrate that EV from canine WJ-MSC significantly disrupted mitogen (ConA)-activated T cell proliferation through biochemical signaling pathways. The data and results described supra suggest that a substantial fraction of the effects of MSC in PBMC assays may arise from insoluble EV-associated factors such as TGF-β and adenosine.

### Wharton's Jelly-MSC (WJ-MSC) Phenotype

The cells isolated from Wharton's Jelly (WJ-MSC) and employed in this Example exhibited surface markers and gene expression that are typical for MSC, such as canine WJ-MSC, including CD44, CD90, and CD105 and the absence of CD45, CD34, and MHCII. Discordance for CD73 and CD34 was observed between gene expression (‘positive’) and protein expression (‘negative’), which may relate to technical issues with antibody reactivity (CD73) and post-transcriptional silencing (CD34). To this point, the collagenase digestion method of WJ-MSC isolation may not have been ideal for isolation of WJ-MSC, since the explant method yields more MSC with greater expansion potential and retention of MSC markers.

WJ-MSC EV Size Distribution and Morphology is Consistent with ‘Small EV’

Minimal criteria for characterization of EV were put forth by a consortium from the International Society of Extracellular Vesicles in 2014 (‘MISEV’), (Lotvall, J. et al., 2014, J. Extracell Vesicles, 3:26913). In the experiments described in this Example, such guidelines were adhered to by detailing EV isolation methods and performing general characterizations (EV-specific and non-EV cellular proteins by Western blot, buoyancy measurements by density gradient and Western blot for TSG101), single vesicle characterization using two methods (TEM and NTA), and functional assays including dose-response, response controls to exclude non-EV (by EV depletions four different ways), and controls for the source of EV (using non-MSC EV). The data and results have rigorously demonstrated that the biological activity measured in PBMC responder assays is based on the use of WJ-MSC EV.

### Interpretation of WJ-MSC EV Phenotype

The particle size distribution and ultrastructural morphology of WJ-MSC EV was consistent with small EV (range ˜50 to 100 nm), but included smaller vesicle-like structures that were not identified. The wide range of EV sizes detected by NTA and TEM in this Example is consistent with the diverse repertoire of vesicles from a single source (Zabeo, D., 2017, J. Extracell. Vesicles, 6(1):1329476). The low buoyancy and detection of TSG101 and Alix implied that canine WJ-MSC EV as isolated for these experiments consisted mainly of small EV (Kourembanas, S., 2015, Annu Rev Physiol., 77:13-27), although canine WJ-MSC EV were larger exosomes isolated from human WJ-MSC that expressed both TSG101 and Alix (Willis, G. R. et al., 2017, Front Cardiovasc Med, 4:63). Differences in isolation and size measurements can make comparisons among studies difficult. Notwithstanding, the method of stepwise ultracentrifugation yielded an EV enriched population of particles for the studies described in this Example.

### WJ-MSC EV Suppress CD4⁺ T Cell Proliferation

Striking T helper cell suppression was observed as a function of EV dosage, a finding that was absent in EV-depleted fractions or in EV from non-MSC fibroblasts. Similarly, MSC EV, including WJ-MSC EV, were strongly suppressive of mitogen stimulated CD4⁺ T cells, but not of those stimulated by mixed lymphocyte reaction (MLR), which led to the use of the mitogen stimulation assay in the experiments described supra. While some have reported that isolated MSC EV are not immunosuppressive in mitogen stimulation assays, even at 10 fold higher concentrations than employed in the assays described here, subtle variations in experimental protocol, as well as other factors (e.g., isolation, purification, handling), may contribute to differences.

The specific observation that canine WJ-MSC EV (or parent WJ-MSC) suppressed the percentage of dividing CD4⁺ T helper cells (responders), but not the number of cell divisions of responders, is consistent with arrest at G0/G1 for suppressed T cells (Hosseinikia, R. et al., 2017, Int J Hematol Oncol Stem Cell Res, 11(1):63-77). That WJ-MSC EV affected CD4⁺ cells to a greater extent than CD4⁻ (CD4^{neg}) cells is a reflection of greater proportions of CD4⁺ than CD4^{neg} mitogen responders in the assays described herein, and not necessarily the specific impact on CD4^{neg} cells. According to the data presented here, the number of EV produced by each MSC is on average approximately 5,000 (5×10³), and the total EV introduced into each PBMC responder well containing 5×10⁵ PBMC is 5×10⁹, or the equivalent EV from 10⁶ MSC. Commonly employed doses of MSC in vivo (2×10⁶/kg or ˜150×10⁶) would effectively suppress T cells within 75 million PBMC. Whether this has any relevance to dose equivalence of biological activity can readily be evaluated by concurrent in vitro and in vivo studies.

Intrinsic variation in the effectiveness (i.e., potency) of WJ-MSC to modulate immune effector cells is a confounding aspect of MSC therapies in regenerative medicine. The range of responses observed across 11 cell lines (only 2 pairs were littermate donors) was explored here. Overall, the responses ranged from 9-92% mean suppression of CD4+ T cells across all WJ-MSC EV lines tested. The major variation was due to two cell lines (9.1%, 14.2% suppression) versus the range across the other 9 cell lines (48-92% suppression). The magnitude observed, under the conditions used, namely, 1 WJ-MSC to 10 PBMC across a transwell, is consistent with reports in the literature (e.g., Barcia, R. N. et al., 2017, Cytotherapy, 19(3):360-370).

“Priming” MSC with a pro-inflammatory stimulus has been shown in various studies to increase their immunosuppressive capacity, and even to increase the capacity of MSC EV to suppress T cells, B cells, and NK cells. However, pretreatment of WJ-MSC with IFNγ over 3 days prior to introduction into transwell assays did not enhance the anti-proliferative activity of WJ-MSC or WJ-MSC EV in the experiments described herein. Similarly, immunosuppression by umbilical cord matrix MSC (human) was not enhanced by IFNγ (Barcia, R. N. et al., 2017, Cytotherapy, 19(3):360-370). Thus, without being bound by theory, IFNγ priming may not be effective in this cell type or in the canine species.

The magnitude of suppression by WJ-MSC EV (50-75%) under the conditions used in the methods described in this Example is comparable to studies using canine AD-MSC or BM-MSC types (see, e.g., Clark, K. et al., 2016, Stem Cell Rev, 12(2):245-256), although direct comparisons are affected by different subsets of lymphocytes stained in these studies.

The presence of TGFβRI on both PBMC and isolated CD4⁺ T cells was demonstrated by Western blot, demonstrating that direct interaction of EV-associated TGF-β with this receptor on CD4⁺ T cells is a plausible mode of action as detected in this assay. In support of this, the addition of 10 or 50 ng/ml, but not 5 ng/ml, TGF-β1 or TGF-β3 suppressed mitogen driven T cell division. Of note, the amount of EV-derived TGF-β1 needed to achieve suppression of CD4⁺ T cell division was approximately 10% of the amount of the soluble form required to produce an equivalent effect (in the assay using 10 ng/mL of recombinant human TGF-β1 (rhTGF-β1). This suggests that EV-associated TGF-β1 may have a heightened, or a qualitatively different, biological activity when compared to soluble TGF-β1. This may be similar to comparisons of EV membrane-associated TGF-β1 derived from cancer-associated fibroblast and dendritic cells versus soluble TGF-β1 (Clayton, A., 2007, Cancer Res, 67(15):7458-7466; Yu, L. et al., 2013, Eur J Immunol, 43(9):2461-2472). The findings that EV associated adenosine had similar effects supports the notion that WJ-MSC EV may introduce a number of mechanisms, similar to the repertoire of growth factors attributed to parent MSC.

The participation of the TGFβIII receptor (betaglycan) was explored, given its role as a co-receptor for TGF-β signaling. Betaglycan can increase affinity for TGF-β to its receptors TGF-βRI and TGF-βRII. TGF-β-induced fibroblast differentiation and angiogenesis by cancer EV require the interaction of betaglycan with TGF-β1 (Webber, J. P. et al., 2015, Oncogene, Vol. 34(3):290-302). The TGF-β1 and betaglycan interaction requires heparin sulfate side chains. In order to determine if a betaglycan interaction capacitated the anti-proliferative effect of WJ-MSC EV on T cells, EV were treated with heparinase as described supra and shown in FIG. 9D. It was observed that heparinase (but not heat inactivated heparinase) reduced EV suppression. These results are consistent with a role for a betaglycan-heparin complex in WJ-MSC EV TGF-β signaling.

The latent form of TGF-β1 was activated to release the mature form of TGF-β1 in the PBMC assay described here. It may also be that EV-derived TGF-β1 is not the major active form in this assay; but rather, non-EV derived sources of TGF-β1, along with other soluble mediators (e.g. IL-10), might be produced de novo in the assay following EV interactions with monocytes or T cells, as an autocrine loop. Knocking down specific cell types, e.g., monocytes, in this assay may resolve the interdependence of cells within the PBMC that are associated with the effect of TGF-β1 and adenosine on T helper cells (CD4+ T cells).

The experiments described in this Example have demonstrated that WJ-MSC EV possess intrinsic mechanisms previously attributed mainly to soluble factors that suppress the proliferation of CD4⁺ T helper cells. As described supra, it was found that WJ-MSC and EV consistently suppressed ConA-induced CD4⁺ T cell proliferation in a dose-dependent fashion, and that the effect was abolished in EV-depleted samples including ultracentrifuge supernatants, EV filtrates, EV from GW4869-pretreated MSC, and Triton-X exposed EV samples. Non-MSC EV did not show lymphocyte suppression. Blockade of TGF-β1 signaling by pretreatment of PBMC with a TGF-βRI antagonist (SB431542) or with neutralizing antibodies to TGF-β1 significantly reduced the anti-proliferative effect of the WJ-MSC EV. Western blotting and ELISA analyses showed that TGF-β was present on WJ-MSC EV in the large latent and pro-form complexes. These data demonstrate that canine WJ-MSC EV are immune modulatory through TGF-β signaling, which may be employed in a method of evaluating the biological potency of cell lines.

### Example 4

### TGF-β in Latent Form on Native WJ-MSC EV

TGF-β was found in a latent form in EV derived from MSC, e.g., WJ-MSC EV. To detect active/mature TGF-β, it was necessary to pre-treat the EV with acid, as shown by ELISA analysis (FIGS. 9A and 9B). As part of the process of obtaining optimally therapeutic or diagnostic EV and for accurately measuring TGF-β in EV, the EV were pre-treated with HCl or similar acid preparations followed by neutralization and measurement using ELISA. Activation can also be achieved by treatment proteases, such as MMP or plasmin; however, protease treatment can disrupt the epitope recognized by antibody in the assay. This finding allow for improving and optimizing the diagnostic or therapeutic applications of EV for use in determining immune status (immunosuppression status), based on cytokines, growth factors, or trophic factors other than TGF-β, for example, PD-L1, Galectin-1, GARP, FasL, CD39/CD73, or integrins.

More specifically, using ELISA analysis, it was determined that the amount of TGF-β1 ranged from 0.1-1.0 ng per 5×10⁹ EV (the number of EV used in each standard PBMC assay well for a 10⁴ EV:PBMC ratio). Some TGF-β1 was detected only after acid activation, consistent with the bulk of TGF-β1 in the latent form associated with EV (FIGS. 9A and 9B). In addition, assessment by Western blot analysis demonstrated that EV-associated TGF-β was detected only at the molecular weight of the large latent complexes (˜150 kDa) and pro-form (˜75 kDa), with the mature homodimer (˜25 kDa) detected only after DTT reduction of those samples (FIG. 9C). In order to determine if beta-glycan served as a functional co-receptor to membranous WJ-MSC EV on T cells, WJ-MSC EV were pre-treated with heparinase, which reduced EV suppression (FIG. 9D). These effects were absent in EV that were pre-treated with heat-inactivated heparinase. The results are consistent with a role for a betaglycan-heparin complex in WJ-MSC EV TGF-β signaling and the activity of membranous TGF-β.

### Example 5

### Hyaluronic Acid/Proteoglycan Complexed to Extracellular Vesicles (EV) Derived from Different Sources and Cell Types and Removed from EV by Treatment with Hyaluronidase

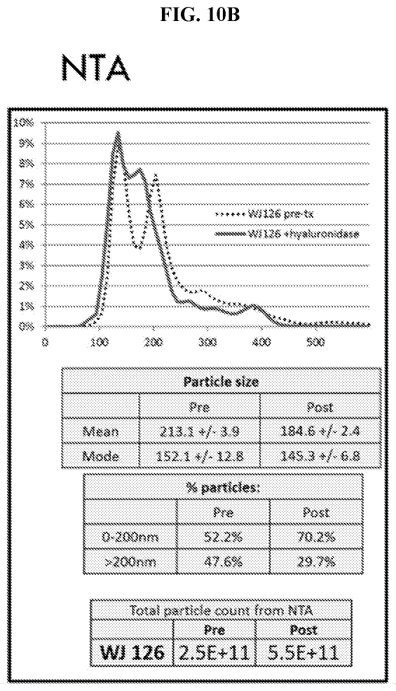

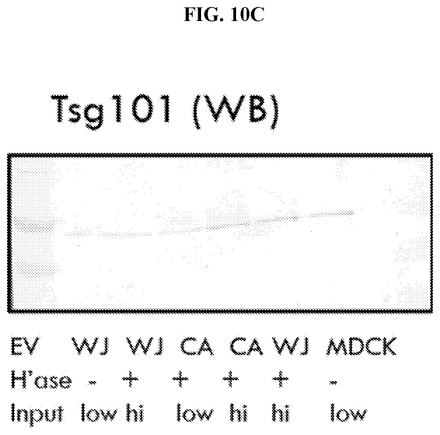

This Example describes the unexpected finding that some EV, e.g., umbilical cord-derived EV, placental-derived EV, or other mesenchymal stem cell-derived EV (or other cell types producing EV) are covered with hyaluronic acid/proteoglycan complexes (or are coated with complexes of hyaluronic acid/proteoglycan). Pre-treatment of such EV with hyaluronidase completely removed those complexes, reducing aggregation and doubling the number of single EV which were available for analysis or treatment, and exposing epitopes including TGF-β. Based on this finding, hyaluronidase is effectively used as a pretreatment of EV preparations, particularly when centrifuged (ultracentrifuged) or concentrated EV preparations are resuspended, e.g., during isolation and manufacture. FIG. 10A shows an SEC-HPLC trace following treatment of a centrifuged sample of WJ-MSC EV with or without hyaluronidase. Treatment with hyaluronidase effectively solubilized individual EV, making their isolation and manufacture more reproducible and resulting in high quality preparations of EV that were reliably benchmarked and analyzed using any downstream technique (e.g., ELISA, Western blots, TEM, vesiculometry, microfluidics, etc.), thereby preventing considerable loss of EV product due to aggregation, e.g., protein aggregation.

Hyaluronidase also dissolves hyaluronic acid in unfractionated synovial fluid, which is too viscous for centrifugation and concentration of EV from that fluid. Amounts of hyaluronidase used to treat synovial fluid, e.g., as provided in Boere, J. et al., 2016, J. Extracell Vesicles, 5:31751, may be used for directly treating EVs covered with hyaluronic acid/proteoglycan complexes. By way of example, an EV preparation may be incubated with 40 μl hyaluronidase (1500 U/mL) for 15 minutes at 37° C. (or room temperature for a longer time period). Unlike the treatment of unfractionated synovial fluid, concentrated, semi-purified or purified EV were optimally treated directly with hyaluronidase as a pretreatment.

FIG. 10B shows the results of NTA, particle size, percent particles and total particle count from NTA, following pretreatment of WJ-MSC EV with or without hyaluronidase. FIG. 10C shows an immunoblot (Western blot) analysis of TSG101 from EV from various MSC treated or untreated with hyaluronidase.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1.-98. (canceled)

99. An isolated extracellular vesicle (EV) comprising transforming growth factor-beta (TGF-13) or an isoform thereof tethered to the membrane surface, wherein the EV is produced by an immortalized cell.

100. The extracellular vesicle (EV) according to claim 1, wherein the immortalized cell is an immune privileged cell selected from the group consisting of umbilical cord, placenta, fetus, testes and articular cartilage.

101. The extracellular vesicle (EV) according to claim 1, wherein the immortalized cell is derived from a stromal cell, stem cell, stromal stem cell, mesenchymal stromal cell (MSC), cancer-associated cell, or fibroblast-like cell.

102. The extracellular vesicle (EV) according to claim 1, wherein the TGF-I3 or isoform thereof is tethered to the membrane of the EV via attachment to one or more of a glycoprotein, P-glycan, or heparin.

102. The extracellular vesicle (EV) according to claim 1, wherein the tethered TGF-I3 is TGF-I31, TGF-r32, TGF-I33, TGF-I34, or a latent form thereof.

104. The extracellular vesicle (EV) according to claim 1, wherein the EV comprises tethered TGF-(3 and at least one other tethered immunomodulatory molecule.

105. The extracellular vesicle (EV) according to claim 6, wherein the at least one other tethered immunomodulatory molecule is selected from PD-1, PD-LI, B7-H4, B7-H5, CTLA-4, 4-1-BB, CD4, CD8, CD14, CD25, CD27, CD40, CD68, CD163, GITR, LAG-3, OX40, TIM3, TIM4, CEA, TLR, TLR2, etc., or cytokines, e.g., IL-4, IL-6, IL-7, IL-10, IL-12, IL-15, IL-17, IFN-γ, Flt3, BLys, chemokines, e.g., CCL21, or Galectin-1.

106. The extracellular vesicle (EV) according to claim 1, wherein the EV comprises an exogenous agent.

107. The extracellular vesicle (EV) according to claim 8, wherein the exogenous agent is a polypeptide, polynucleotide, or small molecule.

108. A method of isolating mesenchymal stromal cell (MSC)-derived extracellular vesicles (EV) having membrane-tethered TGF-I3 (MSC-derived, membrane-tethered TGF-I3 EV), the method comprising: culturing MSC, or a cell or tissue source of MSC, in cell culture or conditioned medium;
isolating the MSC-derived, membrane-tethered TGF-I3 EV from the cell culture or conditioned medium; and optionally, quantifying the amount of MSC-derived, membrane-tethered TGF-I3 EV from the cell or tissue source.

109. The method according to claim 10, wherein cell or tissue source is selected from a biological fluid, umbilical cord tissue, placental tissue, fat, or bone marrow.

110. The method according to claim 10, wherein the MSC are cultured in culture medium for from about 1 day to about 20 days.

11. The method according to claim 10, wherein the culture or conditioned medium is a serum free chemically defined buffered medium, or medium comprised of autologous serum and defined constituents.

112. The method according to claim 10, wherein the MSC-derived, membrane-tethered TGF-f3 EV are isolated by one or more of affinity column chromatography, immune affinity capture, tangential flow filtration, precipitation, differential ultracentrifugation, density gradient centrifugation, or size exclusion chromatography.

113. The method according to claim 10, further comprising quantifying the amount of TGF-P, or a latent form thereof, tethered to the isolated EV having membrane tethered TGF-f3.

114. The method according to claim 15, wherein membrane tethered TGF-f3 is quantified by single vesicle nanoparticle tracking assay, vesiculometry, interferometry, or flow cytometry.

115. A composition for imaging cells or tissue, the composition comprising an extracellular vesicle (EV) according to claim 1, containing an imaging agent.

116. The composition according to claim 17, wherein the imaging agent is a nanoparticle, magnetite, nanoparticle, paramagnetic particle, microsphere, nanosphere, and is selectively targeted to cancer cells.

117. A kit for providing to a subject an extracellular vesicle (EV) derived from mesenchymal stromal cells (MSC) and comprising membrane-tethered TGF-r3 or an isoform thereof (MSC-derived, membrane-tethered TGF-13 EV) as a therapeutic agent, the kit comprising MSC-derived, membrane-tethered TGF-13 EV isolated from MSC.
